(19) United States
(12) Patent Application Publication
(10) Pub. No.: US 2004/0171603 A1
(43) Pub. Date: Sep. 2, 2004

(54) Novel therapeutic targets for the treatment of mycobacterial infections and compounds useful therefor

(76) Inventors: Janos Pato, Budapest (HU); Gyorgy Keri, Budapest (HU); Laszlo Orfi, Budapest (HU); Frigyes Waczek, Budapest (HU); Zoltan Horvath, Budapest (HU); Peter Banhegyi, Budapest (HU); Istavan Szabadkai, Budapest (HU); Jeno Marosfalvi, Budapest (HU); Balint Hegymegi-Barakonyi, Erd (HU); Zsolt Szekelyhidi, Nagykovacsi (HU); Zoltan Greff, Budapest (HU); Axel Choidas, Gauting (DE); Gerald Bacher, Germering (DE); Andrea Missio, Munich (DE); Anil Koul, Munchen (DE)

Correspondence Address: Leon R. Yankwich, YANKWICH & ASSOCIATES, 201 Broadway, Cambridge, MA, 02139, US

(21) Appl. No.: 10/715,591

(22) Filed: Nov. 18, 2003

Related U.S. Application Data
(63) Continuation-in-part of application No. PCT/EP03/03697, filed on Apr. 9, 2003.
(63) Continuation-in-part of application No. PCT/EP02/05573, filed on May 21, 2002.
(60) Provisional application No. 60/292,325, filed on May 22, 2001.
(60) Provisional application No. 60/298,902, filed on Jun. 19, 2001.

(30) Foreign Application Priority Data
May 18, 2001 (EP) 01 112 289.2
Jun. 27, 2001 (EP) 01 115 508.2
Apr. 9, 2002 (EP) 02 007 923.2

(51) Int. Cl. A61K 39/00; A61K 31/43; A61K 31/545

(52) U.S. Cl. 514/192; 514/200

(57) ABSTRACT

Described herein is the discovery that certain mycobacterial serine/threonine protein kinases, particularly protein kinase G (PknG), are effective therapeutic targets for the treatment of mycobacterial infections. Furthermore, the present application refers to the use of mycobacterial serine/threonine protein kinases for developing methods for detection and determination of these kinases for recognizing and monitoring diseases and for controlling therapy of diseases. Additionally disclosed are novel 4,5,6,7-tetrahydrobenzo[b]thiophene compounds, benzo(g)quinoxaline compounds, and pharmaceutically acceptable salts thereof, and methods of using such compounds and salts thereof for the prophylaxis and/or treatment of virally and/or bacterially induced infections, particularly mycobacteria-induced infections, including opportunistic infections, as well as pharmaceutical compositions containing at least one 4,5,6,7-tetrahydrobenzo[b]thiophene compound and/or benzo(g)quinoxaline compound and/or pharmaceutically acceptable salts thereof in a pharmaceutically acceptable carrier.

This application is a continuation-in-part of international application PCT/EP03/03697, filed Apr. 9, 2003 and designating the US, which claims priority to EP application no. 02 007 923.2 filed Apr. 9, 2002, and international application PCT/EP02/05573, filed May 21, 2002 and designating the US, which claims priority to EP application no. 01 112 289.2 filed May 18, 2001, U.S. provisional application No. 60/292,325 filed May 22, 2001, EP application no. 01 115 508.2 filed Jun. 27, 2001, and U.S. provisional application No. 60/298,902 filed Jun. 19, 2001. The contents of the aforementioned applications are incorporated herein by reference.

### FIELD OF THE INVENTION

The present invention relates to the discovery of effective therapeutics for mycobacterial infections and to the discovery that certain serine/threonine protein kinases of mycobacteria, such as mycobacterial serine/threonine protein kinases B, G, and H (PknB, PknG, and PknH), and particularly protein kinase G (PknG) of Mycobacterium tuberculosis, are effective therapeutic targets for the treatment of mycobacterial infections, particularly tuberculosis. The present invention also provides novel compounds useful for the prophylaxis and/or treatment of mycobacteria-induced infections and novel methods for screening for said compounds.

### BACKGROUND OF THE INVENTION

Mycobacteria is the cause of a number of severe diseases, including tuberculosis, leprosy, and mycobacteria-induced meningitis. Tuberculosis is an ancient scourge of human beings, caused by Mycobacterium tuberculosis. Although more than three billion people have been inoculated with the vaccine BCG, presently more than 50,000 people die every week of tuberculosis worldwide, and there are estimates that one third of the world's population is infected by Mycobacterium tuberculosis. According to a recent report of the World Health Organization (WHO) on tuberculosis epidemic, distributed via the internet (http:/www.who.int/inf-fs/en/fact104.html), it is estimated that between the years 2000 and 2020, nearly one billion people will carry tuberculosis bacteria, 200 million people will get sick, and 35 million will die of tuberculosis, if control of the disease and preventive measures are not strengthened. Moreover, it has been reported that 32% of HIV infected individuals die of tuberculosis. The situation has become even more dramatic since a number of Mycobacterium tuberculosis strains have shown a multidrug resistance, which cannot be attacked by conventional therapy, e.g., antibiotics. In addition, immune-suppressed people similar to AIDS patients are often victims of mycobacterial infections leading to a poor prognosis.

There are several reasons why mycobacteria-induced diseases are difficult to cure: First, mycobacteria can perform a differentiation process called “dormancy” or “persistency”. Dormant mycobacteria are much more resistant against conventional antibacterial drug treatments. Second, many of the mycobacteria species have long replication times, resulting in a slow growth. One consequence of this is that antimycobacterial drugs need longer medication times compared to the medication of faster-growing pathogenic bacteria. Both factors cited above are reasons why a medical treatment of mycobacteria-induced diseases has to last at least for several months. A third factor why conventional antibacterial drug treatment is so difficult with regard to mycobacteria-induced diseases is that these bacteria have a relatively thick cell wall, which is impermeable or sparingly permeable for many substances.

Accordingly, new methods for identifying therapeutics that will be effective for the treatment of mycobacterial infections are needed, and additional, improved therapeutics for mycobacterial infections, particularly Mycobacterium tuberculosis infection, are needed.

### SUMMARY OF THE INVENTION

Taking into account the above-mentioned problems with conventional antimycobacterial treatment, it is an object of the present invention to identify novel therapeutic targets for the development of new anti-mycobacterial therapies. It is a further object of the present invention to provide screening assays for discovering novel compounds useful for treating mycobacterial infections. Yet another object of the present invention is to provide compounds and/or pharmaceutically acceptable salts thereof which can be used as pharmaceutically active substances, especially for the prophylaxis and/or treatment of mycobacteria-induced infections; to provide methods to treat mycobacteria-induced diseases by means of those compounds; and to provide compositions comprising at least one of those compounds and/or pharmaceutically acceptable salts thereof as pharmaceutically active ingredients.

To identify substances for drug development against mycobacteria-induced diseases, we searched for inhibitors of signal transduction components present in mycobacteria. As already mentioned above, the elimination of mycobacteria from the human body is presently achieved by inhibiting the growth of respective bacteria by means of antibiotics. According to the present invention, a novel strategy has been used to fight mycobacterial infections, namely to attack mycobacterial signal transduction components which are involved in the persistence of the bacteria within the host cell. Previously, it had been shown that mycobacteria penetrate cells via the endocytotic pathway. Endosomes containing non-pathogenic mycobacteria fuse to lysosomes and subsequently the bacteria are degraded by lysosomal enzymes. However, pathogenic mycobacteria, like Mycobacterium tuberculosis, contain additional “virulence genes” which prevent fusion of endosomes and lysosomes and thus circumvent the degradation mechanism within a host cell.

Mycobacterial serine/threonine protein kinases, particularly Mycobacterium tuberculosis protein kinases (Pkn's) including PknB, PknG, PknH, PknA, PknD, PknE, Pkn F, PknI, PknJ, PknK, and PknL, particularly PknG, have been identified as essential components involved in the persistence and enhanced survival of pathogenic mycobacteria within a macrophage cell line. A particularly important therapeutic target is Mycobacterium tuberculosis serine/threonine protein kinase G (PknG), because it has been surprisingly discovered that the activity of PknG is an essential factor for virulence of Mycobacterium tuberculosis. In accordance with the present invention, compounds have been found which inhibit the activity of PknG in a submicromolar range thus showing that PknG is a suitable target for recognizing diseases, monitoring diseases, and controlling therapy of diseases related to mycobacterial infections. These compounds (inhibitors) were able to induce efficient degradation of mycobacteria within host cells, so that the present invention provides a novel mode for elimination of mycobacteria.

It has been found that certain disease-inducing factors can be secreted by a cellular organism to the environment of the organism. Specifically, in the present invention it has been found that mycobacterial proteins are secreted from the bacterium Mycobacterium tuberculosis to the environment of such a bacterium. One protein which can be secreted by Mycobacterium tuberculosis is the serine/threonine prtotein kinase PknG. The fact that the inventive therapeutic compounds described herein are particularly effective against PknG may be due to the fact that this protein kinase can be attacked by these compounds without the need to penetrate the (thick) cell wall of Mycobacterium tuberculosis. Consequently, the present invention also discloses the use of at least one serine/threonine protein kinase for developing methods for detection and/or determination of these kinases for recognising diseases, for monitoring diseases, and/or for controlling therapy of diseases. Preferably, the methods are immunochemical methods. According to a preferred embodiment of the present invention, the serine/threonine protein kinase used as a target for identifying effective thereapeutics for inhibiting mycobacterial infections is a mycobacterial protein kinase, particularly the mycobacterial serine/threonine protein kinase G (PknG), which is from Mycobacterium tuberculosis.

### BRIEF DESCRIPTION OF THE DRAWINGS

FIG. 1—is a bar graph showing the relative rates of survival of Mycobacterium smegmatis transformed to express wildtype PknG, mutant (non-active) PknG, and empty vector (control) in infected murine macrophage cells. The results indicate the persistence against macrophage degradation pathways of infected cells expressing the wildtype PknG, which demonstrates that PknG is an important virulence factor in mycobacteria.

FIG. 2—is a bar graph illustrating the relative rates of survival of M. smegmatis transformed to express wildtype PknG, mutant (non-active) PknG, and empty vector (control) in infected murine macrophage cells, wherein some of the wildtype PknG-expressing M. smegmatis-infected cells were infected in the presence of 1 μM or 10 μM 2-(Cyclopropanecarbonyl-amino)-4,5,6,7-tetrahydro-benzo[b]thiophene-3-carboxylic acid amide (Compound 237). The results indicate that treatment with the PknG inhibitor Compound 237 overcomes the resistance to macrophage degradation that is characteristic of virulent mycobacterial infection. This validates PknG as a therapeutic target by showing that inhibition of PknG neutralizes the virulence effect of PknG expression.

FIG. 3—is a bar graph illustrating the results of an alkaline phosphatase secretion assay that demonstrates PknG is a secreted kinase.

### DETAILED DESCRIPTION OF THE INVENTION

The present invention relates to a class of compounds discovered to have mycobacterial growth-inhibiting activity is 4,5,6,7-tetrahydrobenzo[b]thiophene and derivatives thereof of formula (I) below. The anti-mycobacterial 4,5,6,7-tetrahydrobenzo[b]thiophene compounds according to the present invention have the general formula (I):

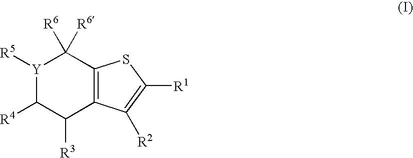

wherein

Y represents C or S;

R¹ represents R², —NH—CO—R¹⁶, —NH₂, —N═CH—R¹⁵, —N═CH—R¹⁶, —NH—CH₂—R¹⁴, —NH—CH₂—R¹⁶, —NH—SO₂—R¹⁴, —NH—CO—NH—R¹⁴, —NH—CS—NH—CH(CCl₃)—NH—CO—R¹⁶, —NH—CH(CCl₃)—NH—CO—R¹², —NH—CS—NH—R¹², N═CH—R¹²,

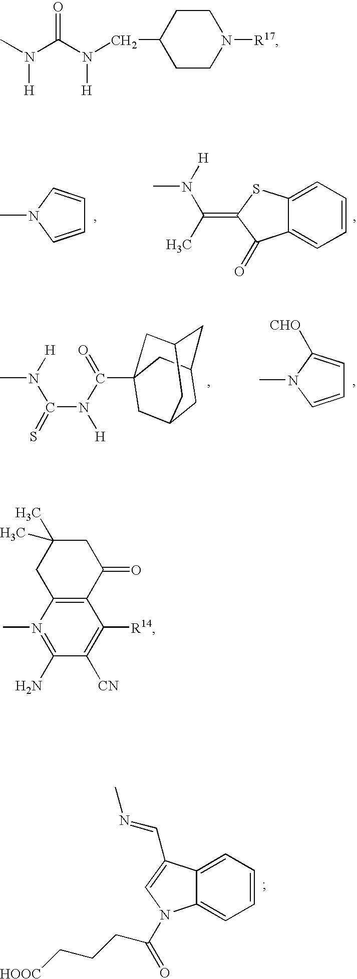

R² represents —COOR¹², —CONR¹²R^{12′}, —CONR¹²R¹⁴; —C≡N, —COCOOR¹², —COCH₂Cl, —COCONHNH₂;

R³, R⁴, R⁵ represent independently of each other —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR¹², —SO₂R¹², —SO₃R¹², —CH₂OR¹²;

in one case R⁶ represents —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR², —SO₂R¹², —SO₃R¹², and R^{6′} is hydrogen;

in the other case R⁶ and R^{6′} together represent a carbonyl oxygen or a oximo residue ═N—OH or ═N—O(O)C—R¹²;

R⁷, R⁸, R⁹, R¹⁰ represent independently of each other —R¹¹, —R¹², —OR¹², —SR¹², —NO₂, —CO—R¹², —COOR¹², —OOCR¹⁶;

R¹¹ represents —F, —Cl, —Br, —I;

R¹², R^{12′} represent independently of each other —H, —CH₃, —C(R¹¹)₃, —C₂H₅, —C₃H₇, —CH(CH₃)₂, —CH₂—CH═CH₂, —CCH₃═CH₂, —CH═C(CH₃)₂, —CH═CH—CH₃, —CH═CHC₂H₅, —CH(CH₃)C₂H₅, —(CH₂)ₙ—C≡C—R⁵, —C₄H₉, —C(CH₃)₃, -Ph, —CH₂—R¹⁵, —C₂H₄OH with n being an integer from 0-2;

R¹³ represents —CF₂CHF₂, —C₅H₁₁, —C₆H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —CH₂SPh, —CH₂R¹¹, —C₂H₄R¹¹, —C₃H₆R¹¹, —C₄H₈R¹¹, —C₂H₄Ph, —CH═CH—COOR¹², —CH₂COOR¹², —C₂H₄COOR¹², —C₃H₆COOR¹², —CH(Ph)-SPh, —C₃H₅, —CH₂CH(Ph)₂, —C₄H₇, —C₅H₉, —C(CH₃)₂CH₂R¹¹, —CH₂CH(CH₃)₂, —CH(R¹¹)Ph, —CH₂CH(CH₃)CH₂C(CH₃)₃, —CH(C₂H₅)—C₄H₉, —CH(R¹¹)₂, —CH(Ph)-C₂H₅, —CH₂C(CH₃)₃,

R¹⁴ represents

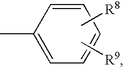

R¹⁵ represents

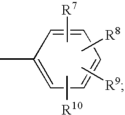

R¹⁶ is R¹²,

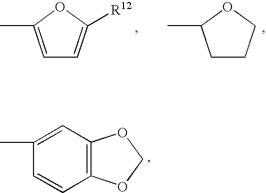

R¹³ R¹⁵

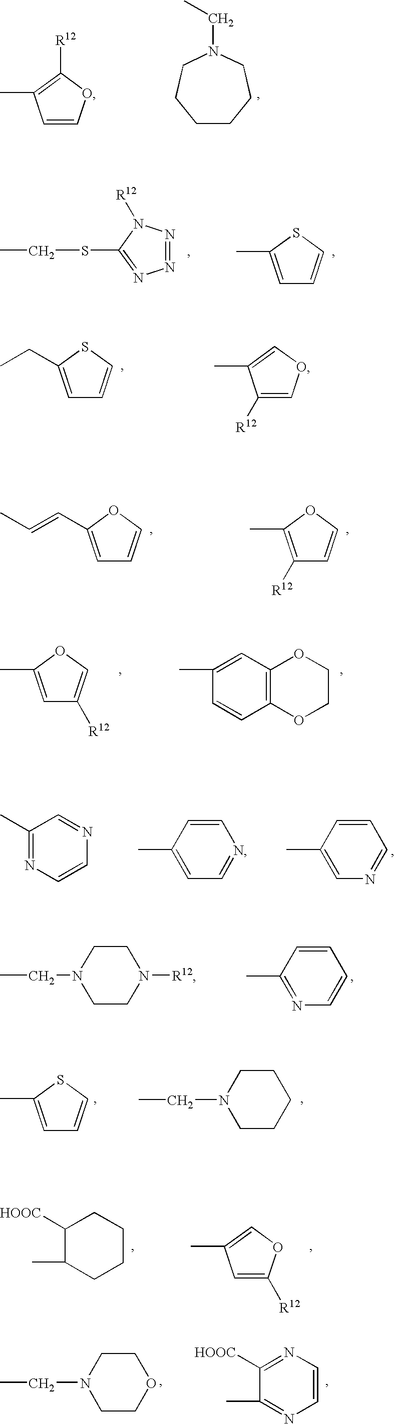

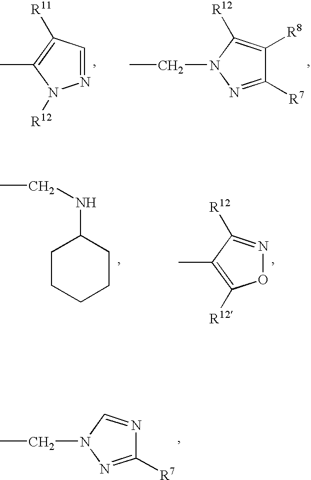

—CH₂O—R¹⁴, —CH═CH—R¹⁴, —CH₂CH(R¹⁴)—CO—R¹², —C₂H₄O—R¹⁴, —C₃H₆O—R¹⁴, —C₂H₄S—CO—R¹², —CH₂S—CO—R¹²; —COOR¹²,

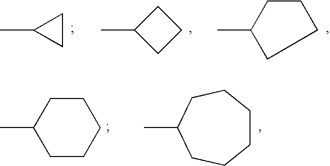

nitrobenzyl, particularly p—nitrobenzyl;

R¹⁷ represents —H, —CO—R¹², —CO—R¹³, —CO—R¹⁴, —CO—NH—R¹², —CO—NH—R¹³, —CO—NH—R¹⁴, —SO₂—R¹⁴, —CO—NH—CH₂—COO—R¹², —CO—CH₂—O—R¹⁴, —CO—CH═CH—R¹⁴,

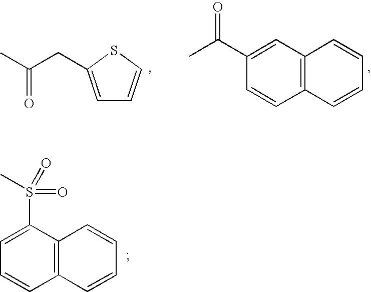

or R¹ and R² together represent a heterocyclic ring system having the following formula

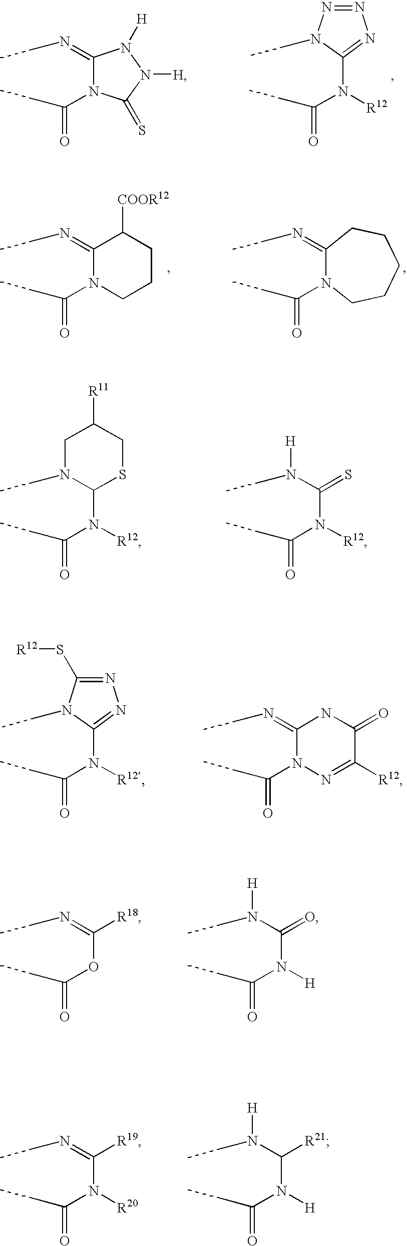

R¹⁸ represents R¹²,

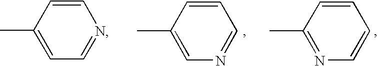

R¹⁹ represents R³, R¹⁴, —SCH₂—R³, —SCH₂—CO—R¹⁴, —SCH₂—CO—NH—R¹⁴, —SCH₂—CO—NH—CH₂—R¹², —NH—CO—CH₂—OR¹⁴, —CO—NH—N═CHR¹²(R¹⁴),

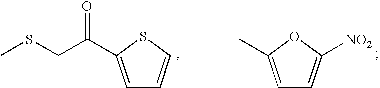

R²⁰ represents R¹², —NH—CO—R¹², —N═CH—R¹⁵;

R²¹ represents R¹⁵,

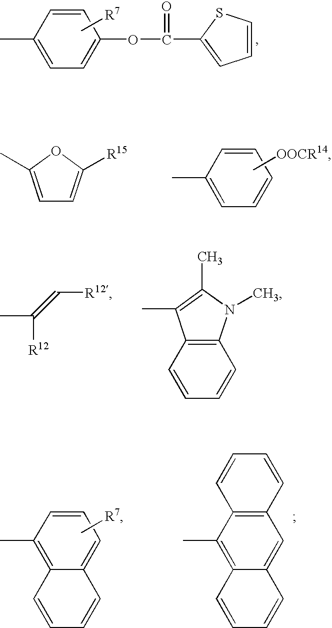

The present invention also comprises pharmaceutically active salts of these 4,5,6,7-tetrahydrobenzo[b]thiophene compounds.

The inventive compounds of the general formula (I) can be synthesized according to the procedures given in WO 99/46267 and WO 01/98290. One synthetic route, for instance, starts from cyclohexanone or cyclohexanone substituted with R³ to R⁶ as defined above which is reacted with alkyl cyanoacetate to the corresponding cyclohexylidene cyano acetic acid alkyl ester. Said ester is converted to the corresponding 4,5,6,7-tetrahydrobenzo[b]thiophene derivative by the reaction with equimolar amounts of sulphur, preferably at temperatures between 40° and 80° C. After having set up the bicyclic tetrahydrobenzo[b]thiophene ring system, the amino and/or carboxyl residues on the thiophene ring may be further modified by esterification or amide bond formation according to standard procedures. Said synthesis may also be accomplished in a combinatorial chemistry fashion with or without the use of a solid support to which one reaction component could be attached.

Preferred are the compounds wherein R¹ is R², —NH—CO—R¹⁶, —NH—CO—NH—R¹⁴, —N═CH—R¹⁵, —NH—CH₂—R¹⁴, —NH—SO₂—R¹⁴, —NH—CS—NH—CH(CCl₃)—NH—CO—R¹⁶, —NH—CH(CCl₃)—NH—CO—R¹², —NH—CS—NH—R¹²,

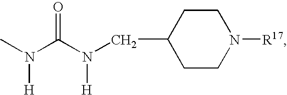

and wherein R², R¹², R¹⁴, R¹⁵, R¹⁶, and R¹⁷ have the meanings as defined above in the general formula (I).

Also Preferred are compounds wherein R³, R⁴, R⁵ and R⁶ represent independently of each other —R¹¹, —R¹², —R^{12′}, —OR¹², —SR¹², —NO₂, —CO—R¹², —COOR¹², —CONR¹²R^{12′}, —NR¹²R^{12′}, —SO₂R¹², —SO₃R¹², —CH₂O R¹², and wherein R¹¹, R¹², and R^{12′} have the meanings as defined above in the general formula (I).

Another preferred subgroup of compounds of the general formula (I) is the group wherein R⁷, R⁸, R⁹, R¹⁰ represent independently of each other —R¹¹, —R¹², —OR¹², —SR¹², —NO₂, —CO—R¹², —COOR¹², —OOCR¹⁶, and wherein R¹¹ and R¹² have the meanings as defined above in the general formula (I).

Furthermore, compounds of the general formula (I) are preferred wherein R¹³ represents —CF₂CHF₂, —C₅H₁₁, —C₆H₁₁, —C₆H₁₃, —C₇H₁₅, —C₈H₁₇, —C₁₀H₂₁, —C₁₁H₂₃, —C₁₂H₂₅, —C₁₃H₂₇, —CH₂SPh, —CH₂R¹¹, —C₂H₄R¹¹, —C₃H₆R¹¹, —C₄H₈R¹¹, —C₂H₄Ph, —CH═CH—COOR¹², —CH(R¹¹)₂, —CH₂COOR¹², —C₂H₄COOR¹², —C₃H₆COOR¹², —CH(Ph)-SPh, —C₃H₅, —CH₂CH(Ph)₂, —C₄H₇, —C₅H₉, —C(CH₃)₂CH₂R¹¹, —CH₂CH(CH₃)₂, —CH(R¹¹)Ph, —CH₂CH(CH₃)₂CH₂C(CH₃)₃, —CH(C₂H₅)—C₄H₉, —CH(Ph)-C₂H₅, —CH₂C(CH₃)₃, and wherein R¹¹ and R¹² have the meanings as defined above in the general formula (I).

The following compounds are also advantageous wherein

R¹⁶ represents R¹², R¹³, R¹⁵,

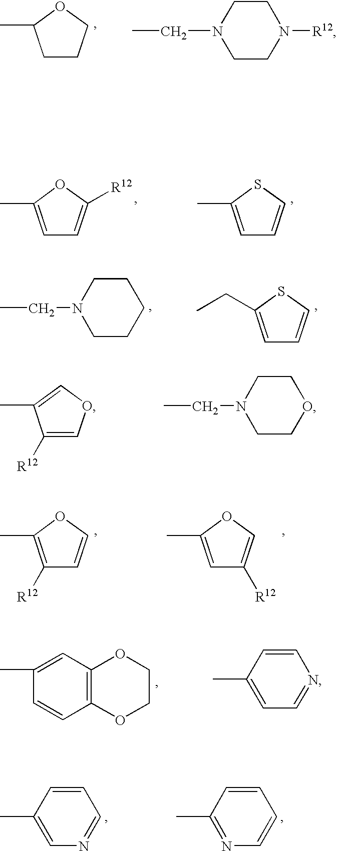

and wherein R¹², R¹³, and R¹⁵ have the meanings as defined above in the general formula (I).

Also preferred is the group of compounds wherein R¹ and R² form a heterocyclic ring system having the following formulas

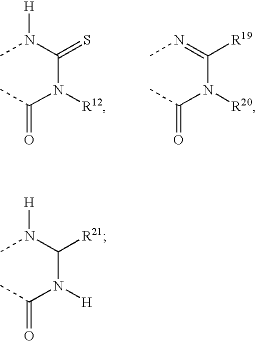

and wherein R¹², R¹⁹, R²⁰, and R²¹ have the meanings as defined above in the general formula (I).

Particularly preferred are the compounds selected from the group consisting of:

(Compound 1) 2-{3-[1-(2-Chloro-acetyl)-piperidin-4-ylmethyl]-ureido}4,5,6,7-tetrahydro-benzo[b]thiophene-3-carboxylic acid ethyl ester;

(Compound 2) 2-[3-(1-Butyryl-piperidin-4-ylmethyl)-ureido]-4,5,6,7-tetrahydro-benzo[b]thiophene-3-carboxylic acid ethyl ester;

(Compound 3) 2-[3-(1-Propanoyl-piperidin-4-ylmethyl)-ureido]-4,5,6,7-tetrahydro-benzo[b]thiophene-3-carboxylic acid ethyl ester;

(Compound 4) 2-[3-(1-Benzoyl-piperidin-4-ylmethyl)-ureido]-4,5,6,7-tetrahydro-benzo[b]thiophene-3-carboxylic acid ethyl ester;

(Compound 5) 2-{3-[1-(2-Chloro-benzoyl-piperidin-4-ylmethyl]-ureido}-4,5,6,7-tetrahydro-benzo[b]thiophene-3-carboxylic acid ethyl ester;

(Compound 6) 2-[3-(1-Isobutyryl-piperidin-4-ylmethyl)-ureido]-4,5,6,7-tetrahydro-benzo[b]thiophene-3-carboxylic acid ethyl ester;

(Compound 7) 2-{3-[1-(4-Methyl-benzoyl-piperidin-4-ylmethyl]-ureido}-4,5,6,7-tetrahydro-benzo[b]thiophene-3-carboxylic acid ethyl ester;

(Compound 8) 2-[3-(1-Cyclohexanecarbonyl-piperidin-4-ylmethyl)-ureido]-4,5,6,7-tetrahydro-benzo[b]thiophene-3-carboxylic acid ethyl ester;

(Compound 9) 2-[3-(1-Cyclopropanecarbonyl-piperidin-4-ylmethyl)-ureido]-4,5,6,7-tetrahydro-benzo[b]thiophene-3-carboxylic acid ethyl ester;

(Compound 10) 2-[3-(1-Hexanoyl-piperidin-4-ylmethyl)-ureido]-4,5,6,7-tetrahydro-benzo[b]thiophene-3-carboxylic acid ethyl ester;

(Compound 11) 2-{3-[1-(2-Methyl-benzoyl-piperidin-4-ylmethyl]-ureido}-4,5,6,7-tetrahydro-benzo[b]thiophene-3-carboxylic acid ethyl ester;

(Compound 12) 2-{3-[1-(3-Chloro-2,2-dimethyl-propionyl)-piperidin-4-ylmethyl]-ureido}-4,5,6,7-tetrahydro-benzo[b]thiophene-3-carboxylic acid ethyl ester;

(Compound 13) 2-{3-[1-(3,5,5-Trimethyl-hexanoyl-piperidin-4-ylmethyl]-ureido}-4,5,6,7-tetrahydro-benzo[b]thiophene-3-carboxylic acid ethyl ester;

(Compound 14) 2-{3-[1-(2-Ethyl-hexanoyl-piperidin-4-ylmethyl]-ureido}-4,5,6,7-tetrahydro-benzo[b]thiophene-3-carboxylic acid ethyl ester;

(Compound 15) 2-{3-[1-(2-Phenyl-butyryl-piperidin-4-ylmethyl]-ureido}-4,5,6,7-tetrahydro-benzo[b]thiophene-3-carboxylic acid ethyl ester;

(Compound 16) 2-[3-(1-Cyclopentylcarbonyl-piperidin-4-ylmethyl)-ureido]-4,5,6,7-tetrahydro-benzo[b]thiophene-3-carboxylic acid ethyl ester;

(Compound 17) 2-{3-[1-(2-Chloro-2-phenyl-acetyl-piperidin-4-ylmethyl]-ureido}-4,5,6,7-tetrahydro-benzo[b]thiophene-3-carboxylic acid ethyl ester;

(Compound 18) 2-{3-[1-(4-Butyl-benzoyl-piperidin-4-ylmethyl]-ureido}-4,5,6,7-tetrahydro-benzo[b]thiophene-3-carboxylic acid ethyl ester;

(Compound 19) 2-[3-(1-Decanoyl-piperidin-4-ylmethyl)-ureido]-4,5,6,7-tetrahydro-benzo[b]thiophene-3-carboxylic acid ethyl ester;

(Compound 20) 2-[3-(1-Heptanoyl-piperidin-4-ylmethyl)-ureido]-4,5,6,7-tetrahydro-benzo[b]thiophene-3-carboxylic acid ethyl ester;

(Compound 21) 2-[3-(1-Nonanoyl-piperidin-4-ylmethyl)-ureido]-4,5,6,7-tetrahydro-benzo[b]thiophene-3-carboxylic acid ethyl ester;

(Compound 22) 2-[3-(1-Dodecanoyl-piperidin-4-ylmethyl)-ureido]-4,5,6,7-tetrahydro-benzo[b]thiophene-3-carboxylic acid ethyl ester;

(Compound 23) 2-{3-[1-(3-Methy-butyryl)-piperidin-4-ylmethyl]-ureido}-4,5,6,7-tetrahydro-benzo[b]thiophene-3-carboxylic acid ethyl ester;

(Compound 24) 2-[3-(1-Tetradecanoyl-piperidin-4-ylmethyl)-ureido]-4,5,6,7-tetrahydro-benzo[b]thiophene-3-carboxylic acid ethyl ester;

(Compound 25) 2-[3-(1-Cyclohexylcarbamoyl-piperidin-4-ylmethyl)-ureido]-4,5,6,7-tetrahydro-benzo[b]thiophene-3-carboxylic acid ethyl ester;

(Compound 26) 2-[3-(1-Phenylcarbamoyl-piperidin-4-ylmethyl)-ureido]-4,5,6,7-tetrahydro-benzo[b]thiophene-3-carboxylic acid ethyl ester;

(Compound 27) 2-[3-(1-Benzylcarbamoyl-piperidin-4-ylmethyl)-ureido]-4,5,6,7-tetrahydro-benzo[b]thiophene-3-carboxylic acid ethyl ester;

(Compound 28) 2-{3-[1-(4-Ethoxycarbonyl-phenylcarbamoyl)-piperidin-4-ylmethyl]-ureido}-4,5,6,7-tetrahydro-benzo[b]thiophene-3-carboxylic acid ethyl ester;

(Compound 29) 2-{3-[1-(3-Bromo-phenylcarbamoyl)-piperidin-4-ylmethyl]-ureido}-4,5,6,7-tetrahydro-benzo[b]thiophene-3-carboxylic acid ethyl ester;

(Compound 30) 2-{3-[1-(2-Methoxy-phenylcarbamoyl)-piperidin-4-ylmethyl]-ureido}-4,5,6,7-tetrahydro-benzo[b]thiophene-3-carboxylic acid ethyl ester;

(Compound 31) 2-{3-[1-(2-Methyl-phenylcarbamoyl)-piperidin-4-ylmethyl]-ureido}-4,5,6,7-tetrahydro-benzo[b]thiophene-3-carboxylic acid ethyl ester;

(Compound 32) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;

(Compound 33) 2-{3-[1-(4-Fluoro-benzenesulfonyl)-piperidin-4-ylmethyl]-ureido}-4,5,6,7-tetrahydro-benzo[b]thiophene-3-carboxylic acid ethyl ester;

(Compound 34) 2-{3-[1-(4-Methyl-benzenesulfonyl)-piperidin-4-ylmethyl]-ureido}-4,5,6,7-tetrahydro-benzo[b]thiophene-3-carboxylic acid ethyl ester;

(Compound 35) 2-{3-[1-Naphtalene-1-sulfonyl)-piperidin-4-ylmethyl]-ureido}-4,5,6,7-tetrahydro-benzo[b]thiophene-3-carboxylic acid ethyl ester;

(Compound 36) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;

(Compound 37) 2-{3-[1-(2,5-Dichloro-benzenesulfonyl)-piperidin-4-ylmethyl]-ureido}-4,5,6,7-tetrahydro-benzo[b]thiophene-3-carboxylic acid ethyl ester;

(Compound 38) 2-{3-[1-(2,2-Dichloro-acetyl)-piperidin-4-ylmethyl]-ureido}-4,5,6,7-tetrahydro-benzo[b]thiophene-3-carboxylic acid ethyl ester;

(Compound 39) 2-{3-[1-(3,3-Dimethy-butyryl)-piperidin-4-ylmethyl]-ureido}-4,5,6,7-tetrahydro-benzo[b]thiophene-3-carboxylic acid ethyl ester;

(Compound 40) 2-{3-[1-(Ethoxycarbonylmethyl-carbamoyl)-piperidin-4-ylmethyl]-ureido}-4,5,6,7-tetrahydro-benzo[b]thiophene-3-carboxylic acid ethyl ester;

(Compound 41) 2-{3-[1-(3-Methoxy-phenylcarbamoyl)-piperidin-4-ylmethyl]-ureido}-4,5,6,7-tetrahydro-benzo[b]thiophene-3-carboxylic acid ethyl ester;

(Compound 42) 2-{3-[1-(3,5-Bis-trifluoromethyl-benzoyl)-piperidin-4-ylmethyl]-ureido}-4,5,6,7-tetrahydro-benzo[b]thiophene-3-carboxylic acid ethyl ester;

(Compound 43) 2-[3-(1-Phenylacetyl-piperidin-4-ylmethyl]-ureido]-4,5,6,7-tetrahydro-benzo[b]thiophene-3-carboxylic acid ethyl ester;

(Compound 44) 2-{3-[1-(3,4-Dichloro-benzoyl-piperidin-4-ylmethyl]-ureido}-4,5,6,7-tetrahydro-benzo[b]thiophene-3-carboxylic acid ethyl ester;

(Compound 45) 2-{3-[1-(2,4,6-Trimethyl-benzenesulfonyl)-piperidin-4-ylmethyl]-ureido}-4,5,6,7-tetrahydro-benzo[b]thiophene-3-carboxylic acid ethyl ester;

(Compound 46) 2-{3-[1-(4-Methoxy-benzenesulfonyl)-piperidin-4-ylmethyl]-ureido}-4,5,6,7-tetrahydro-benzo[b]thiophene-3-carboxylic acid ethyl ester;

(Compound 47) 2-{3-[1-(Naphtalene-2-sulfonyl)-piperidin-4-ylmethyl]-ureido}-4,5,6,7-tetrahydro-benzo[b]thiophene-3-carboxylic acid ethyl ester;

(Compound 48) 2-(3-Piperidin-4-ylmethyl-ureido)-4,5,6,7-tetrahydro-benzo[b]thiophene-3-carboxylic acid ethyl ester;

(Compound 49) 2-(3-{1-[2-(4-Chloro-phenoxy)-acetyl]-piperidin-4-ylmethyl}-ureido)-4,5,6,7-tetrahydro-benzo[b]thiophene-3-carboxylic acid ethyl ester;

(Compound 50) 2-(3-{1-[3-(4-Nitro-phenoyl)-acryloyl]-piperidin-4-ylmethyl}-ureido)-4,5,6,7-tetrahydro-benzo[b]thiophene-3-carboxylic acid ethyl ester;

(Compound 51) 2-[3-(1-Octanoyl-piperidin-4-ylmethyl)-ureido]-4,5,6,7-tetrahydro-benzo[b]thiophene-3-carboxylic acid ethyl ester;

(Compound 52) 2-[3-(1-Benzenesulfonyl-piperidin-4-ylmethyl)-ureido]-4,5,6,7-tetrahydro-benzo[b]thiophene-3-carboxylic acid ethyl ester;

(Compound 53) 2-[3-(1-Ethylylcarbamoyl-piperidin-4-ylmethyl)-ureido]-4,5,6,7-tetrahydro-benzo[b]thiophene-3-carboxylic acid ethyl ester;

(Compound 54) 2-{3-[1-(3-Fluoro-benzoyl-piperidin-4-ylmethyl]-ureido}-4,5,6,7-tetrahydro-benzo[b]thiophene-3-carboxylic acid ethyl ester;

(Compound 55) 2-{3-[1-(4-Chloro-phenylcarbamoyl)-piperidin-4-ylmethyl]-ureido}-4,5,6,7-tetrahydro-benzo[b]thiophene-3-carboxylic acid ethyl ester;

(Compound 56) 4-[3-(3-ethoxycarbonyl-4,5,6,7-tetrahydro-benzo[b]thiophene-2-yl)-ureidomethyl]-piperidne-1-carboxylic acid phenyl ester;

(Compound 57) 2-{3-[1-(3-Trifluoromethyl-benzoyl-piperidin-4-ylmethyl]-ureido}-4,5,6,7-tetrahydro-benzo[b]thiophene-3-carboxylic acid ethyl ester;

(Compound 58) 2-{3-[1-(3-Bromo-benzoyl-piperidin-4-ylmethyl]-ureido}-4,5,6,7-tetrahydro-benzo[b]thiophene-3-carboxylic acid ethyl ester;

(Compound 59) 2-{3-[1-(2,4-Dichloro-phenylcarbamoyl)-piperidin-4-ylmethyl]-ureido}-4,5,6,7-tetrahydro-benzo[b]thiophene-3-carboxylic acid ethyl ester;

(Compound 60) 2-{3-[1-(2-Trifluoromethyl-phenylcarbamoyl)-piperidin-4-ylmethyl]-ureido}-4,5,6,7-tetrahydro-benzo[b]thiophene-3-carboxylic acid ethyl ester;

(Compound 61) 2-{3-[1-(4-tert-Butyl-benzoyl-piperidin-4-ylmethyl]-ureido}-4,5,6,7-tetrahydro-benzo[b]thiophene-3-carboxylic acid ethyl ester;

(Compound 62) 2-{3-[1-(3-Nitro-benzoyl-piperidin-4-ylmethyl]-ureido}-4,5,6,7-tetrahydro-benzo[b]thiophene-3-carboxylic acid ethyl ester;

(Compound 63) 2-[3-(1-Cyclobutanecarbonyl-piperidin-4-ylmethyl)-ureido]-4,5,6,7-tetrahydro-benzo[b]thiophene-3-carboxylic acid ethyl ester;

(Compound 64) 2-{3-[1-(2-Thiophen-2-ylacetyl)-piperidin-4-ylmethyl]-ureido}-4,5,6,7-tetrahydro-benzo[b]thiophene-3-carboxylic acid ethyl ester;

(Compound 65) 2-{3-[1-(Naphthalene-2-carbonyl)-piperidin-4-ylmethyl]-ureido}-4,5,6,7-tetrahydro-benzo[b]thiophene-3-carboxylic acid ethyl ester;

(Compound 66) 5,6,7,8-Tetrahydro-1H-benzol[4,5]thieno[2,3-d]pyrimidine-2,4-dione;

(Compound 67) 4-Oxo-3,4,5,6,7,8-hexahydro-benzo[4,5]thieno[2,3-d]pyrimidine-2-carboxylic acid ethyl ester;

(Compound 68) 2-Acetylamino-4,5,6,7-tetrahydro-benzo[b]thiophene-3-carboxylic acid ethyl ester;

(Compound 69) 2-Amino-4,5,6,7-tetrahydro-benzo[b]thiophene-3-carboxylic acid ethyl ester;

(Compound 70) 2-Amino-4,5,6,7-tetrahydro-benzo[b]thiophene-3-carboxylicacid amide;

(Compound 71) 2-Acetylamino-4,5,6,7-tetrahydro-benzo[b]thiophene-3-carboxylic acid;

(Compound 72) 2-(Toluene-4-sulfonylamino)-4,5,6,7-tetrahydro-benzo[b]thiophene-3-carboxylic acid amide;

(Compound 73) 7-Methyl-1,3,4,9-tetrahydro-2H-11-thia-5a,6,9,10-tetraaza-benzo[b]fluorene-5,8-dione;

(Compound 74) 7-Phenyl-1,3,4,9-tetrahydro-2H-11-thia-5a,6,9,10-tetraaza-benzo[b]fluorene-5,8-dione;

(Compound 75) 2-(5-Nitro-furan-2-yl)-2,3,5,6,7,8-hexahydro-1H-benzo[4,5]thieno[2,3-d]pyrimidin-4-one;

(Compound 76) 2-[(5-Bromo-2-hydroxy-benzylidene)-amino]-4,5,6,7-tetrahydro-benzo[b]thiophene-3-carboxylic acid amide;

(Compound 77) 2-[(4,5-Dibromo-2-hydroxy-benzylidene)-amino]-4,5,6,7-tetrahydro-benzo[b]thiophene-3-carboxylic acid ethyl ester;

(Compound 78) 2-[(2-Chloro-benzylidene)-amino]-4,5,6,7-tetrahydro-benzo[b]thiophene-3-carboxylic acid amide;

(Compound 79) 2-[1-(3-Oxo-3H-benzo[b]thiophen-2-ylidene)-ethylamino]-4,5,6,7-tetrahydro-benzo[b]thiophene-3-carboxylic ethyl ester;

(Compound 80) 2-Heptanoylamino-4,5,6,7-tetrahydro-benzo[b]thiophene-3-carboxylic acid ethyl ester;

(Compound 81) 2-(3-Bromo-benzoylamino)-4,5,6,7-tetrahydro-benzo[b]thiophene-3-carboxylic acid ethyl ester;

(Compound 82) 2-Ethyl-5,6,7,8-tetrahydro-3-oxa-9-thia-1-aza-fluoren-4-one;

(Compound 83) Furan-2-carboxylic acid [3-(4-methoxy-phenylcarbamoyl)-4,5,6,7-tetrahydro-benzo[b]thiophen-2-yl]-amide;

(Compound 84) 2-Pyridin-3-yl-5,6,7,8-tetrahydro-3-oxa-9-thia-1-aza-fluoren-4-one;

(Compound 85) 2-Propionylamino-4,5,6,7-tetrahydro-benzo[b]thiophene-3-carboxylic phenylamide;

(Compound 86) 1-Allylsulfanyl-4-phenyl-6,7,8,9-tetrahydro-4H-10-thia-2,3,4,10b-tetraaza-cyclopenta[a]fluoren-5-one;

(Compound 87) 2-[2-(1-Phenyl-1H-tetrazol-5-ylsulfanyl)-acetylamino]-4,5,6,7-tetrahydro-benzo[b]thiophene-3-carboxylic acid ethyl ester;

(Compound 88) 2-[3-(4-Methoxy-phenyl)-3-(2,2,2-trifluoro-acetylamino)-propionyl-amino]-4,5,6,7-tetrahydro-benzo[b]thiophene-3-carboxylic acid amide;

(Compound 89) 2-(3-Chloro-benzyolamino)-4,5,6,7-tetrahydro-benzo[b]thiophene-3-carboxylic acid ethyl ester;

(Compound 90) 2-{[1-(4-Carboxy-butyryl)-1H-indol-3-ylmethylene]-amino}-4,5,6,7-tetrahydro-benzo[b]thiophene-3-carboxylic acid ethyl ester;

(Compound 91) 2-(7-Ethyl-4-oxo-3-phenyl-3,4,5,6,7,8-hexahydro-benzo)[4,5]thieno [2,3-d]pyrimidin-2-ylsulfanyl)-N-(2-isopropoxy-phenyl)-acetamide;

(Compound 92) N,N-Diethyl-2-(7-ethyl-4-oxo-3-phenyl-3,4,5,6,7,8-hexahydro-benzo)[4,5]thieno[2,3-d]pyrimidin-2-ylsulfanyl)-acetamide;

(Compound 93) 3-[(4-Hydroxy-3-methoxy-benzylidene)-amino]-2-methyl-5,6,7,8-tetrahydro-3H-benzo[4,5]thieno[2,3-d]pyrimidin-4-one;

(Compound 94) 6-Ethyl-2-(3-phenyl-thioureido)-4,5,6,7-tetrahydro-benzo[b]thiophene-3-carboxylic acid ethyl ester;

(Compound 95) 2-[3-(Adamantane-1-carbonyl)-thioureido]-4,5,6,7-tetrahydro-benzo[b]thiophene-3-carboxylic acid ethyl ester;

(Compound 96) 2-[(3,5-Dibromo-2,4-dihydroxy-benzylidene)-amino]-4,5,6,7-tetrahydro-benzo[b]thiophene-3-carboxylic acid ethyl ester;

(Compound 97) Benzoic acid 2-ethoxy-4-(4-oxo-1,2,3,4,4a,5,6,7,8,9a-decahydro-benzo [4,5]thieno[2,3-d]pyrimidin-2-yl)-phenylester;

(Compound 98) 2-(1-Acetylamino-2,2,2-trichloro-ethylamino)-4,5,6,7-tetrahydro-benzo[b]thiophene-3-carboxylic acid ethyl ester;

(Compound 99) 2-(3,5-Di-tert-butyl-4-hydroxy-phenyl)-2,3,5,6,7,8-hexahydro-1H-benzo[4,5]thieno[2,3-d]pyrimidin-4-one;

(Compound 100) 4-Oxo-3,4,5,6,7,8-hexahydro-benzo[4,5]thieno[2,3-d]pyrimidine-2-carboxylic acid [1-(3-nitro-phenyl)-ethylidene]-hydrazide;

(Compound 101) 2-[(Pyridine-4-carbonyl)-amino]-4,5,6,7-tetrahydro-benzo[b]thiophene-3-carboxylic acid ethyl ester;

(Compound 102) 2-(2,2,3,3-Tetrafluro-propionylamino)-4,5,6,7-tetrahydro-benzo[b]thiophene-3-carboxylic acid amide;

(Compound 103) 2-[3-(1-Acetylamino-2,2,2-trichloro-ethyl)-thioureido]4,5,6,7-tetrahydro-benzo[b]thiophene-3-carboxylic acid ethyl ester;

(Compound 104) 2-[3-(2,2,2-Trichloro-1-propionylamino-ethyl)-thioureido]-4,5,6,7-tetrahydro-benzo[b]thiophene-3-carboxylic acid amide;

(Compound 105) 2-(3-Chloro-phenyl)-2,3,5,6,7,8-hexahydro-1H-benzo[4,5]thieno[2,3-d]pyrimidin-4-one;

(Compound 106) 2-(2-Chloro-phenyl)-2,3,5,6,7,8-hexahydro-1H-benzo[4,5]thieno[2,3-d]pyrimidin-4-one;

(Compound 107) 2-Anthracene-9-yl-2,3,5,6,7,8-hexahydro-1H-benzo[4,5]thieno[2,3-d]pyrimidin-4-one;

(Compound 108) 2-(3,5-Dibromo-2-methoxy-phenyl)-2,3,5,6,7,8-hexahydro-1H-benzo[4,5]thieno[2,3-d]pyrimidin-4-one;

(Compound 109) 2-[5-(4-Bromo-phenyl)-furan-2-yl]-2,3,4a,5,6,7,8,9a-octahydro-1H-benzo[4,5]thieno[2,3-d]pyrimidin-4-one;

(Compound 110) 2-(2,4-Dichlorophenoxy)-N-(4-oxo-2-propyl-5,6,7,8-tetrahydro-4H-benzo[4,5]thieno [2,3-d]pyrimidin-3-yl)-acetamide;

(Compound 111) 2-(3,4-Dimethoxy-benzoylamino)-4,5,6,7-tetrahydro-benzo[b]thiophene-3-carboxylic acid ethyl ester;

(Compound 112) 2-[3-(2-Chloro-phenyl)-acryloylamino]-4,5,6,7-tetrahydro-benzo[b]thiophene-3-carboxylic acid ethyl ester;

(Compound 113) 3-(3-Ethoxycarbonyl-4,5,6,7-tetrahydrobenzo[b]thiophene-2-ylcarbamoyl)-pyrazine-2-carboxylic acid;

(Compound 114) 2-(3-{2,2,2-Trichloro-1-[(furan-2-carbonyl)-amino]-ethyl}-thioureido)-4,5,6,7-tetrahydro-benzo[b]thiophene-3-carboxylic acid ethyl ester;

(Compound 115) 2-(1-Methyl-2-phenyl-vinyl)-2,3,4a,5,6,7,8,9a-octahydro-1H-benzo[4,5]thieno[2,3-d]pyrimidin-4-one;

(Compound 116) 2-(2-Methoxy-naphtalen-1-yl)-2,3,4a,5,6,7,8,9a-octahydro-1H-benzo[4,5]thieno[2,3-d]pyrimidin-4-one;

(Compound 117) 2-(1,2-Dimethyl-1H-indol-3-yl)-2,3,4a,5,6,7,8,9a-octahydro-1H-benzo[4,5]thieno[2,3-d]pyrimidin-4-one;

(Compound 118) 2-(Cyclohexanecarbonyl-amino)-6-methyl-4,5,6,7-tetrahydro-benzo[b]thiophene-3-carboxylic acid ethyl ester;

(Compound 119) 3-Bromo-benzoic acid 3-(4-oxo-1,2,3,4,5,6,7,8-octahydro-benzo[4,5]thieno[2,3-d]pyrimidin-2-yl)-phenyl ester;

(Compound 120) 3-Thioxo-2,3,5,6,7,8-hexahydro-1H-9-thia-1,2,3a,10-tetraaza-cyclopenta[b]fluorene-4-one;

(Compound 121) 2-(2,3-Dibromo-5-ethoxy-4-hydroxy-phenyl)-2,3,5,6,7,8-hexahydro-1H-benzo[4,5]thieno[2,3-d]pyrimidin-4-one;

(Compound 122) 2-[2-(4-Chloro-phenoxy)-acetylamino]-6-methyl-4,5,6,7-tetrahydro-benzo[b]thiophene-3-carboxylic acid ethyl ester;

(Compound 123) 4-Phenyl-5a,6,7,8,9,10a-hexahydro-4H-10-thia-1,2,3,4,10b-pentaaza-cyclopenta[a]fluoren-5-one;

(Compound 124) 6-Methyl-2-[(thiophene-2-carbonyl)-amino]-4,5,6,7-tetrahydro-benzo[b]thiophene-3-carboxylic acid amide;

(Compound 125) 3-Allyl-2-(2-oxo-2-thiophen-2-yl-ethylsulfanyl)-5,6,7,8-tetrahydro-3H-benzo[4,5]thieno[2,3-d]pyrimidin-4-one;

(Compound 126) 2-[(2-Chloro-4-nitro-benzylidene)-amino]-4,5,6,7-tetrahydro-benzo[b]thiophene-3-carboxylic acid ethyl ester;

(Compound 127) 2-(2-Methyl-3,5-dinitro-benzoylamino)-4,5,6,7-tetrahydro-benzo[b]thiophene-3-carboxylic acid ethyl ester;

(Compound 128) 2-(4-Acetyl-benzoylamino)-4,5,6,7-tetrahydro-benzo[b]thiophene-3-carboxylic acid ethyl ester;

(Compound 129) 2-Benzoylamino-7-hydroxyimino-4,5,6,7-tetrahydro-benzo[b]thiophene-3-carboxylic acid ethyl ester;

(Compound 130) 2-Formylamino-4,5,6,7-tetrahydro-benzo[b]thiophene-3-carboxylic acid ethyl ester;

(Compound 131) 5-Oxo-1,2,3,4,6,7,8,9-octahydro-5H-11-thia-5a,10-diaza-benzo[b]fluo-rene-9-carboxylic acid ethyl ester;

(Compound 132) 2-Benzoylamino-7-oxo-4,5,6,7-tetrahydro-benzo[b]thio-phene-3-carboxylic acid ethyl ester;

(Compound 133) 2-(2-Phenylsulfanyl-acetylamino)-4,5,6,7-tetrahydro-benzo[b]thiophene-3-carboxylic acid ethyl ester;

(Compound 134) 2-(4-Nitro-benzylamino)-4,5,6,7-tetrahydro-benzo[b]thiophene-3-carb-oxylic acid ethyl ester;

(Compound 135) 2-[2-(4-Ethyl-piperazin-1-yl)-acetylamino]-7-oxo-4,5,6,7-tetrahydro-benzo[b]thiophene-3-carboxylic acid ethyl ester;

(Compound 136) 2-Phenylsulfanyl-3,4,7,8,9,10-hexahydro-2H,6H-12-thia-5,11-diaza-cyclohepta[b]fluorene-1,5-dione;

(Compound 137) 7-Hydroxy-2-(4-nitro-benzoylamino)-4,5,6,7-tetrahydro-benzo[b]thio-phene-3-carboxylic acid ethyl ester;

(Compound 138) 2-(2-Cyclohexylamino-acetylamino)-7-oxo-4,5,6,7-tetrahydro-benzo[b]thiophene-3-carboxylic acid ethyl ester;

(Compound 139) 2-(2-Azepan-1-yl-acetylamino)-7-oxo-4,5,6,7-tetrahydro-benzo[b]thio-phene-3-carboxylic acid ethyl ester;

(Compound 140) 7-Hydroxyimino-2-pentanoylamino-4,5,6,7-tetrahydro-benzo[b]thiophene-3-carboxylic acid ethyl ester;

(Compound 141) 2-(2-Morpholin-4-yl-acetylamino)-7-oxo-4,5,6,7-tetrahydro-benzo[b]thiophene-3-carboxylic acid ethyl ester;

(Compound 142) 2-Benzoyloxyimion-4,5,7,8,9,10,11-heptahydro-3H-1-thia-6a,12-diaza-cyclohepta[b]fluorene-6-one;

(Compound 143) 2-(3-Acetylsulfanyl-propionylamino)-4,5,6,7-tetrahydro-benzo[b]thio-phene-3-carboxylic acid ethyl ester;

(Compound 144) 2-[3-(3,4-Dichloro-phenyl-ureido]4,5,6,7-tetrahydro-benzo[b]thiophene-3-carboxylic acid amide;

(Compound 145) 2-(2-Chloro-5-iodo-benzoylamino)-4,5,6,7-tetrahydro-benzo[b]thiophene-3-carboxylic acid ethyl ester;

(Compound 146) 2-[(5-Benzyl-2-hydroxy-3-nitro-benzylidene)-amino]-4,5,6,7-tetrahydro-benzo[b]thiophene-3-carboxylic acid ethyl ester;

(Compound 147) 2-[(Pyridine-3-carbonyl)-amino]-4,5,6,7-tetrahydro-benzo[b]thiophene-3-carboxylic acid ethyl ester;

(Compound 148) 2-(3-Thiophen-2-yl-propionylamino)-4,5,6,7-tetrahydro-benzo[b]thiophene-3-carboxylic acid ethyl ester;

(Compound 149) 6-Methyl-2-(3-methyl-4-nitro-benzoylamino)-4,5,6,7-tetrahydro-benzo[b]thiophene-3-carboxylic acid ethyl ester;

(Compound 150) 2-Isopropyl-5,6,7,8-tetrahydro-3-oxa-9-thia-1-aza-fluor-4-one;

(Compound 151) 2-(2-Piperidin-1-yl-acetylamino)-4,5,6,7-tetrahydro-benzo[b]thiophene-3-carboxylic acid amide;

(Compound 152) 3-Allyl-2-prop-2-ynylsulfanyl-5,6,7,8-tetrahydro-3H-benzo[4,5]thieno[2,3-d]pyrimidin-4-one;

(Compound 153) 2-(Trifluoromethyl)-5,6,7,8-tetrahydro-3H-benzo[4,5]thieno[2,3-d]pyrimidin-4-one;

(Compound 154) 2-[2-(4-Methyl-piperazin-1-yl)-acetylamino]-4,5,6,7-tetrahydro-benzo[b]thiophene-3-carboxylic acid amide;

(Compound 155) Furan-2-carboxylic acid-(3-phenylcarbamoyl-4,5,6,7-tetrahydro-benzo[b]thiophen-2-yl)-amide;

(Compound 156) 3-Phenyl-5,6,7,8-tetrahydro-3H-benzo[4,5]thieno[2,3-d]pyrimidin-4-one;

(Compound 157) 2-(4-Oxo-3-phenyl-3,4,5,6,7,8-hexahydro-benzo[4,5]thieno[2,3-d]pyrimidin-2-ylsulfanyl)-N-phenethyl-acetamide;

(Compound 158) 2-(3-Benzoylsulfanyl-acetylamino)-4,5,6,7-tetrahydro-benzo[b]-thiophene-3-carboxylic acid ethyl ester;

(Compound 159) 2-(5-Hydroxy-2-nitro-phenyl)-2,3,5,6,7,8-hexahydro-1H-benzo[4,5]thieno[2,3-d]pyrimidin-4-one;

(Compound 160) 2-[2-(2-Benzoylamino-2-carboxy-ethylsulfanyl)-acetylamino]-4,5,6,7-tetrahydro-benzo[b]thiophene-3-carboxylic acid ethyl ester;

(Compound 161) 2-[(5-Bromoo-2-hydroxy-benzylidene)-amino]-4,5,6,7-tetrahydro-benzo[b]thiophene-3-carboxylic acid ethyl ester;

(Compound 162) 3-[(2,4-Dihydroxy-benzylidene)-amino]-2-methyl-5,6,7,8-tetrahydro-3H-benzo[4,5]thieno[2,3-d]pyrimidin-4-one;

(Compound 163) N-(4-Oxo-2-pentyl-5-pentyl-5,6,7,8-tetrahydro-4H-benzo[4,5]thieno[2,3-d]pyrimidin-3-yl)-2-phenyl-acetamide;

(Compound 164) 2-[(2,3-Dihydro-benzo[1,4]dioxine-5-carbonyl)-amino]-4,5,6,7-tetrahydro-benzo[b]thiophene-3-carboxylic acid ethyl ester;

(Compound 165) 2-[(3,5-Dichloro-4-methoxy-benzylidene)-amino]-4,5,6,7-tetrahydro-benzo[b]thiophene-3-carboxylic acid ethyl ester;

(Compound 166) 2-Iodo-5-phenyl-2,3,4a,5,6,7,8,9,10-octahydro-1H-4,11-dithia-5,11b-diaza-benzo[a]fluoren-6-one;

(Compound 167) 2-Benzoylamino-4,5,6,7-tetrahydro-benzo[b]thiophene-3-carboxylic acid;

(Compound 168) 2-(2,2,2-Trichloro-1-phenylacetylamino-ethylamino)-4,5,6,7-tetrahydro-benzo[b]thiophene-3-carboxylic acid ethyl ester;

(Compound 169) 2-(3-Furan-2-yl-acryloylamino)-4,5,6,7-tetrahydro-benzo[b]thiophene-3-carboxylic acid amide;

(Compound 170) 3-[(3-Bromo-4-hydroxy-5-methoxy-benzylidene)-amino]-2-methyl-5,6,7,8-tetrahydro-3H-benzo[4,5]thieno [2,3-d]pyrimidin-4-one;

(Compound 171) 3-[(3-Chloro-4-hydroxy-5-methoxy-benzylidene)-amino]-2-methyl-5,6,7,8-tetrahydro-3H-benzo[4,5]thieno[2,3-d]pyrimidin-4-one;

(Compound 172) 2-[3-(4-Chloro-2-methyl-phenoxy)-propionylamino]-4,5,6,7-tetrahydro-benzo[b]thiophene-3-carboxylic acid methyl ester;

(Compound 173) 2-Amino-6-methyl-4,5,6,7-tetrahydro-benzo[b]thiophene-3-carboxylic acid isopropyl ester;

(Compound 174) 7-Benzoyloxyimino-2-pyrrol-1-yl-4,5,6,7-tetrahydro-benzo[b]thiophene-3-carboxylic acid ethyl ester;

(Compound 175) 2-(2-Formyl-pyrrol-1-yl)-4,5,6,7-tetrahydro-benzo[b]thiophene-3-carboxylic acid;

(Compound 176) 2-(5-Chloro-pentanoylamino)-4,5,6,7-tetrahydro-benzo[b]thiophene-3-carboxylic acid ethyl ester;

(Compound 177) 3-Allyl-6-methyl-2-thioxo-2,3,5,6,7,8-hexahydro-1H-benzo[4,5]thieno[2,3-d]pyrimidin-4-one;

(Compound 178) (3-Allyl-6-methyl-4-oxo-3,4,5,6,7,8-hexahydro-benzo[4,5]thieno[2,3-d]pyrimidin-2-ylsulfanyl)-acetic acid;

(Compound 179) 2-[2-(4-Bromo-phenyl-2-oxo-ethylsulfanyl)]-6-methyl-3-phenyl-5,6,7,8-tetrahydro-3H-benzo[4,5]thieno[2,3-d]pyrimidin-4-one;

(Compound 180) 2-Amino-5-methyl-4,5,6,7-tetrahydro-benzo[b]thiophene-3-carboxylic acid ethyl ester;

(Compound 181) 2-(2-Bromo-benzoylamino)-7-oxo-4,5,6,7-tetrahydro-benzo[b]thiophene-3-carboxylic acid ethyl ester;

(Compound 182) 2-(3-Methyl-benzyolamino)-4,5,6,7-tetrahydro-benzo[b]thiophene-3-carboxylic acid ethyl ester;

(Compound 183) 2-(4-Benzoyl-benzoylamino)-4,5,6,7-tetrahydro-benzo[b]thiophene-3-carboxylic acid ethyl ester;

(Compound 184) 2-[(2-Ethoxy-benzylidene)-amino]-4,5,6,7-tetrahydro-benzo[b]thiophene-3-carboxylic acid amide;

(Compound 185) 2-[2-Amino-3-cyano-7,7-dimethyl-4-(3-nitro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;

(Compound 186) 2-[2-Amino-3-cyano-7,7-dimethyl-4-(4-chloro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;

(Compound 187) 2-[2-Amino-3-cyano-7,7-dimethyl-4-(4-ethyl-phenyl)-5-oxo-4a,5,6,7,8,8a-hexahydro-4H-quinolin-1-yl]-4,5,6,7-tetrahydro-benzo[b]thiophene-3-carboxylic acid ethyl ester;

(Compound 188) 2-[2-Amino-3-cyano-7,7-dimethyl-4-(4-nitro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;

(Compound 189) (3-Allyl-4-oxo-3,4,5,6,7,8-hexahydro-benzo[4,5]thieno[2,3-d]pyrimidin-2-ylsulfanyl)-acetic acid methyl ester;

(Compound 190) 2-(2-Hydroxy-ethylsulfanyl)-5,6,7,8-tetrahydro-3H-benzo[4,5]thieno[2,3-d]pyrimidin-4-one;

(Compound 191) Thiophene-2-carboxylic acid 2-ethoxy-4-(4-oxo-3,4,5,6,7,8-hexahydro-benzo-[4,5]thieno-[2,3-d]pyrimidin-2-ylsulfanyl)-phenyl ester;

(Compound 192) 2-(2-Fluoro-benzoylamino)-6-methyl-4,5,6,7-tetrahydro-benzo[b]thiophene-3-carboxylic acid isopropyl ester;

(Compound 193) 3a,7a-Dihydro-benzo[1,3]dioxole-5-carboxylic acid (3-carbamoyl-5-methyl-4,5,6,7-tetrahydro-benzo[b]thiophen-2-yl)-amide;

(Compound 194) 2-[(3a,7a-Dihydro-benzo[1,3]dioxole-5-carbonyl)-amino]-6-methyl-4,5,6,7-tetrahydro-benzo[b]thiophene-3-carboxylic acid isopropyl ester;

(Compound 195) 2-[(3a,7a-Dihydro-benzo[1,3]dioxole-5-carbonyl)-amino]-4,5,6,7-tetrahydro-benzo[b]thiophene-3-carboxylic acid isopropyl ester;

(Compound 196) 6-Methyl-2-(3-phenyl-propionylamino)-4,5,6,7-tetrahydro-benzo[b]thiophene-3-carboxylic acid amide;

(Compound 197) 2-(2,4-Dichloro-benzoylamino)-6-Methyl-4,5,6,7-tetrahydro-benzo[b]thiophene-3-carboxylic acid isopropyl ester;

(Compound 198) 2-(4-Methoxy-benzoylamino)-4,5,6,7-tetrahydro-benzo[b]thiophene-3-carboxylic acid methyl ester;

(Compound 199) Tetrahydro-furan-2-carboxylic acid (3-carbamoyl-6-methyl-4,5,6,7-tetrahydro-benzo[b]thiophen-2-yl)-amide;

(Compound 200) 2-[2-(5-Methyl-3-nitro-pyrazol-1-yl)-acetylamino]-4,5,6,7-tetrahydro-benzo[b]thiophene-3-carboxylic acid methyl ester;

(Compound 201) 2-(4-Fluoro-benzenesulfonylamino)-4,5,6,7-tetrahydro-benzo[b]thiophene-3-carboxylic acid amide;

(Compound 202) 6-tert-Butyl-2-(3-phenyl-3-phenylsulfanyl-propionylamino)-4,5,6,7-tetrahydro-benzo[b]thiophene-3-carboxylic acid methyl ester;

(Compound 203) 2-[2-(3,5-Dimethyl-4-nitro-pyrazol-1-yl)-acetylamino]-6-methyl-4,5,6,7-tetrahydro-benzo[b]thiophene-3-carboxylic acid ethyl ester;

(Compound 204) 2-[2-(3,5-Dimethyl-4-nitro-pyrazol-1-yl)-acetylamino]-4,5,6,7-tetrahydro-benzo[b]thiophene-3-carboxylic acid isopropyl ester;

(Compound 205) 6-Methyl-2-(3,3-diphenyl-propionylamino)-4,5,6,7-tetrahydro-benzo[b]thiophene-3-carboxylic acid ethyl ester;

(Compound 206) 2-(3,5-Dimethoxy-benzoylamino)-6-(1,1-dimethyl-propyl)-4,5,6,7-tetrahydro-benzo[b]thiophene-3-carboxylic acid methyl ester;

(Compound 207) 6-tert-Butyl-2-[2-(5-methyl-3-trifluormethyl-pyrazol-1-yl)-acetyl-amino]-4,5,6,7-tetrahydro-benzo[b]thiophene-3-carboxylic acid amide;

(Compound 208) 6-tert-Butyl-2-(3-phenyl-3-phenylsulfanyl-propionylamino)-4,5,6,7-tetrahydro-benzo[b]thiophene-3-carboxylic acid ethyl ester;

(Compound 209) 2-[2-(5-Methyl-3-nitro-pyrazol-1-yl)-acetylamino]-4,5,6,7-tetrahydro-benzo[b]thiophene-3-carboxylic acid isopropyl ester;

(Compound 210) 6-tert-Butyl-2-[2-(3-nitro-[1,2,4]triazol-1-yl)-acetylamino]-4,5,6,7-tetrahydro-benzo[b]thiophene-3-carboxylic acid methyl ester;

(Compound 211) 6-tert-Butyl-2-[2-(3,5-dimethyl-4-nitro-pyrazol-1-yl)-acetylamino]-4,5,6,7-tetrahydro-benzo[b]thiophene-3-carboxylic acid methyl ester;

(Compound 212) 6-Methyl-2-[2-(5-methyl-3-trifluoromethyl-pyrazol-1-yl)-acetylamino]-4,5,6,7-tetrahydro-benzo[b]thiophene-3-carboxylic acid ethyl ester;

(Compound 213) 6-Methyl-2-[2-(5-methyl-3-trifluoromethyl-pyrazol-1-yl)-acetylamino]-4,5,6,7-tetrahydro-benzo[b]thiophene-3-carboxylic acid methyl ester;

(Compound 214) 6-tert-Butyl-2-[2-(5-methyl-3-trifluoromethyl-pyrazol-1-yl)-acetylamino]-4,5,6,7-tetrahydro-benzo[b]thiophene-3-carboxylic acid ethyl ester;

(Compound 215) 6-tert-Butyl-2-[2-(3,5-dimethyl-4-nitro-pyrazol-1-yl)-acetylamino]-4,5,6,7-tetrahydro-benzo[b]thiophene-3-carboxylic acid amide;

(Compound 216) 6-tert-Butyl-2-(3-carboxy-acryloylamino)-4,5,6,7-tetrahydro-benzo[b]thiophene-3-carboxylic acid methyl ester;

(Compound 217) 6-tert-Butyl-2-(4-carboxy-butyrylamino)-4,5,6,7-tetrahydro-benzo[b]thiophene-3-carboxylic acid methyl ester;

(Compound 218) 2-[(4-Iodo-2-methyl-2H-pyrazole-3-carbonyl)-amino]-4,5,6,7-tetrahydro-benzo[b]thiophene-3-carboxylic acid ethyl ester;

(Compound 219) 2-[4-(4-Chloro-2-methyl-phenoxy)-butyrylamino]-6-methyl-4,5,6,7-tetrahydro-benzo[b]thiophene-3-carboxylic acid isopropyl ester;

(Compound 220) 2-(2-Phenyl-2-phenylsulfanyl-acetylamino)-4,5,6,7-tetrahydro-benzo[b]thiophene-3-carboxylic acid ethyl ester;

(Compound 221) 2-[(5-Methyl-3-phenyl-isoxazole-4-carbonyl)-amino]-4,5,6,7-tetrahydro-benzo[b]thiophene-3-carboxylic acid ethyl ester;

(Compound 222) 6-Methyl-2-[2-(3-nitro-[1,2,4]triazol-1-yl)-acetylamino]-4,5,6,7-tetrahydro-benzo[b]thiophene-3-carboxylic acid methyl ester;

(Compound 223) 6-Methyl-2-[2-(3-nitro-[1,2,4]triazol-1-yl)-acetylamino]-4,5,6,7-tetrahydro-benzo[b]thiophene-3-carboxylic acid isopropyl ester;

(Compound 224) 2-Amino-6-tert-butyl-4,5,6,7-tetrahydro-benzo[b]thiophene-3-carbox-ylic acid methyl ester;

(Compound 225) 2-Amino-6-tert-butyl-4,5,6,7-tetrahydro-benzo[b]thiophene-3-carbox-ylic acid amide;

(Compound 226) 6-Methyl-2-[2-(5-methyl-3-nitro-pyrazol-1-yl)-acetylamino]-4,5,6,7-tetrahydro-benzo[b]thiophene-3-carboxylic acid methyl ester;

(Compound 227) 2-[2-(3,5-Dimethyl-4-nitro-pyrazol-1-yl)-acetylamino]-4,5,6,7-tetrahydro-benzo[b]thiophene-3-carboxylic acid methyl ester;

(Compound 228) 2-[2-(3,5-Dimethyl-4-nitro-pyrazol-1-yl)-acetylamino]-4,5,6,7-tetrahydro-benzo[b]thiophene-3-carboxylic acid amide;

(Compound 229) 2-[2-(5-Methyl-3-trifluoromethyl-pyrazol-1-yl)-acetylamino]-4,5,6,7-tetrahydro-benzo[b]thiophene-3-carboxylic acid methyl ester;

(Compound 230) 6-Methyl-2-[2-(3-nitro-[1,2,4]triazol-1-yl)-acetylamino]-4,5,6,7-tetrahydro-benzo[b]thiophene-3-carboxylic acid ethyl ester;

(Compound 231) 2-[(2-Carboxy-cyclohexanecarbonyl)-amino]-4,5,6,7-tetrahydro-benzo[b]thiophene-3-carboxylic acid isopropyl ester;

(Compound 232) 2-(3-Carboxy-acryloylamino)-6-(1,1-dimethyl-propyl)-4,5,6,7-tetrahydro-benzo[b]thiophene-3-carboxylic acid amide;

(Compound 233) 6-(1,1-Dimethyl-propyl)-2-[(5-methyl-furan-2-carbonyl)-amino]-4,5,6,7-tetrahydro-benzo[b]thiophene-3-carboxylic acid amide;

(Compound 234) 6-Methyl-2-[(2-methyl-furan-3-carbonyl)-amino]-4,5,6,7-tetrahydro-benzo[b]thiophene-3-carboxylic acid isopropyl ester;

(Compound 235) 2-(4-Chloro-benzoylamino)-4,5,6,7-tetrahydro-benzo[b]thiophene-3-carboxylic acid ethyl ester;

(Compound 236) 2-Benzoylamino-4,5,6,7-tetrahydro-benzo[b]thiophene-3-carboxylic acid ethyl ester;

(Compound 237) 2-(Cyclopropanecarbonyl-amino)-4,5,6,7-tetrahydro-benzo[b]thiophene-3-carboxylic acid amide;

(Compound 238) 2-[2-(4-Nitrophenyl)-acetylamino]-4,5,6,7-tetrahydro-benzo[b]thiophene-3-carboxylic acid amide;

(Compound 239) 6-Methyl-2-[2-(3-nitro-[1,2,4]triazol-1-yl)-acetylamino]-4,5,6,7-tetrahydro-benzo[b]thiophene-3-carboxylic acid amide;

(Compound 240) Furan-2-carboxylic acid [3-(2-hydroxy-ethylcarbamoyl)-4,5,6,7-tetrahydro-benzo[b]thiophen-2-yl]-amide;

(Compound 241) Cyclopropanecarboxylic acid (3-ethoxy-4,5,6,7-tetrahydro-benzo[b]thiophen-2-yl)-amide;

(Compound 242) N-(3-Ethoxy-4,5,6,7-tetrahydro-benzo[b]thiophen-2-yl)-acet-amide;

(Compound 243) N-(3-Ethoxy-4,5,6,7-tetrahydro-benzo[b]thiophen-2-yl)-propionamide;

(Compound 244) N-(3-Ethoxy-4,5,6,7-tetrahydro-benzo[b]thiophen-2-yl)-2-methyl-acrylamide;

(Compound 245) 3-Methyl-but-2-enoic acid (3-ethoxy-4,5,6,7-tetrahydro-benzo[b]thiophen-2-yl)-amide;

(Compound 246) But-2-enoic acid (3-ethoxy-4,5,6,7-tetrahydro-benzo[b]thiophen-2-yl)-amide;

(Compound 247) N-(3-ethoxy-4,5,6,7-tetrahydro-benzo[b]thiophen-2-yl)-2,2-dimethyl-propionamide;

(Compound 248) Thiophene-2-carboxylic acid (3-ethoxy-4,5,6,7-tetrahydro-benzo[b]thiophen-2-yl)-amide;

(Compound 249) Furan-2-carboxylic acid (3-ethoxy-4,5,6,7-tetrahydro-benzo[b]thiophen-2-yl)-amide;

(Compound 250) 2-Acetylamino-4,5,6,7-tetrahydrobenzo[b]thiophene-3-carboxylic acid amide;

(Compound 251) 2-(2,2,-Dimethyl-propionylamino)-4,5,6,7-tetrahydrobenzo[b]thiophene-3-carboxylic acid amide;

(Compound 252) 2-(Cyclopropanecarbonyl-amino)-4,5,6,7-tetrahydro-benzo[b]thiophene-3-carboxylic acid;

(Compound 253) Cyclopropanecarboxylic acid (3-cyano-4,5,6,7-tetrahydro-benzo[b]thiophen-2-yl)amide;

(Compound 254) 2-Amino-4,5,6,7-tetrahydro-benzo[b]thiophene-3-carbonitrile;

(Compound 255) 2-Isobutyrylamino-4,5,6,7-tetrahydrobenzo[b]thiophene-3-carboxylic acid amide;

(Compound 256) 2-Isobutyrylamino-4,5,6,7-tetrahydrobenzo[b]thiophene-3-carboxylic acid ethyl ester;

(Compound 257) 2-(Ethoxyoxalyl-amino)-4,5,6,7-tetrahydrobenzo[b]thiophene-3-carboxylic acid ethyl ester;

(Compound 258) 2-Amino-4-methyl-4,5,6,7-tetrahydrobenzo[b]thiophene-3-carboxylic acid ethyl ester;

(Compound 259) 2-(Cyclopropanecarbonyl amino)-4-methyl-4,5,6,7-tetrahydro-benzo[b]thiophene-3-carboxylic acid ethyl ester;

(Compound 260) Oxo-(4,5,6,7-tetrahydrobenzo[b]thiophen-2-yl)-acetic acid;

(Compound 261) 2-Chloro-1-(4,5,6,7-tetrahydrobenzo[b]thiophen-2-yl)-ethanone;

(Compound 262) 2-Hydrazino-1-(4,5,6,7-tetrahydrobenzo[b]thiophen-2-yl)-ethanone;

(Compound 263) 2-(2-Methyl-acryloylamino)-4,5,6,7-tetrahydrobenzo[b]thiophene-3-carboxylic acid amide;

(Compound 264) 2-[(Thiophene-2-carbonyl)-amino]-4,5,6,7-tetrahydrobenzo[b]thiophene-3-carboxylic acid amide;

(Compound 265) Furan-2-carboxylic acid (3-carbamoyl-4,5,6,7-tetrahydro-benzo[b]-thiophen-2-yl)-amide;

(Compound 266) 2-(Cyclobutanecarbonyl-amino)-4,5,6,7-tetrahydrobenzo[b]thiophene-3-carboxylic acid amide;

(Compound 267) 2-(2-Methyl-butyrylamino)-4,5,6,7-tetrahydrobenzo[b]thiophene-3-carboxylic acid amide;

(Compound 268) 2-(Cyclopropanecarbonyl-amino)-4-methyl-4,5,6,7-tetrahydrobenzo[b]thiophene-3-carboxylic acid amide;

(Compound 269) 6-tert-Butyl 2-(cyclopropanecarbonyl-amino)-4,5,6,7-tetrahydrobenzo[b]thiophene-3-carboxylic acid amide;

(Compound 270) 2-(Cyclopropanecarbonyl-amino)-6-methyl-4,5,6,7-tetrahydrobenzo[b]thiophene-3-carboxylic acid amide;

(Compound 271) 2-(Cyclopropylmethyl-amino)-4,5,6,7-tetrahydrobenzo[b]thiophene-3-carboxylic acid amide;

(Compound 272) N-(3-Cyano-4,5,6,7-tetrahydrobenzo[b]thiophen-2-yl)-acetamide;

(Compound 273) N-(3-Cyano-4,5,6,7-tetrahydrobenzo[b]thiophen-2-yl)-benzamide;

(Compound 274) 5-Bromo-furan-2-carboxylic acid (3-cyano-4,5,6,7-tetrahydrobenzo[b]thiophen-2-yl)-amide;

(Compound 275) N-(3-Cyano-4,5,6,7-tetrahydrobenzo[b]thiophen-2-yl)-2,2,2,-trifluoro-acetamide;

(Compound 276) 2-[(Furan-2-ylmethylene)-amino]-4,5,6,7-tetrahydrobenzo[b]thiophene-3-carbonitrile;

(Compound 277) N-(3-Cyano-6-methyl-4,5,6,7-tetrahydrobenzo[b]thiophen-2-yl)acetamide;

(Compound 278) 2-[(Pyrazine-2-carbonyl)-amino]-4,5,6,7-tetrahydrobenzo[b]thiophene-3-carboxylic acid methyl ester;

(Compound 279) 2-Isobutyrylamino-4,5,6,7-tetrahydrobenzo[b]thiophene-3-carboxylic acid methyl ester;

(Compound 280) 6-Methyl-2-[(thiophene-2-carbonyl)-amino]-4,5,6,7-tetrahydrobenzo[b]thiophene-3-carboxylic acid;

(Compound 281) 2-[(Thiophene-2-carbonyl)-amino]-4,5,6,7-tetrahydrobenzo[b]thiophene-3-carboxylic acid;

(Compound 282) 2-(Cyclopropanecarbonyl-amino) 4,5,6,7-tetrahydrobenzo[b]thiophene-3-carboxylic acid;

(Compound 283) 2-(Cyclohexanecarbonyl-amino) 4,5,6,7-tetrahydrobenzo[b]thiophene-3-carboxylic acid amide;

(Compound 284) 2-Acetylamino-6-methyl-4,5,6,7-tetrahydrobenzo[b]thiophene-3-carboxylic acid amide;

(Compound 285) 2-Amino-4,7-dihydro-5H-thieno[2,3-c]-thiopyran-3-carboxylic acid amide;

(Compound 286) 2-(Cyclopropanecarbonyl-amino)-4,7-dihydro-5H-thieno[2,3-c]-thiopyran-3-carboxylic acid amide;

(Compound 287) 2-(Cyclopropanecarbonyl-amino)-6λ⁴-oxo-4,5,6,7-tetrahydro-thieno[2,3-c]thiopyran-3-carboxylic acid amide.

Other aspects of the present invention relate to 4,5,6,7-tetrahydrobenzo[b]thiophene compounds of the general formula (I) as shown above as new pharmaceutically active agents, particularly for the prophylaxis and/or treatment of mycobateria-induced infections (including opportunistic infections) and diseases, to pharmaceutical compositions comprising these 4,5,6,7-tetrahydrobenzo[b]thiophene derivatives as active ingredients, and to a method for treating virally and/or bacterially induced diseases, particularly mycobacteria-induced infections, in mammals, including humans. Surprisingly, it was found that 4,5,6,7-tetrahydrobenzo[b]thiophene compounds of the general formula (I) as well as pharmaceutically acceptable salts of these compounds are effective against mycobacteria-induced infections and diseases at pharmaceutically acceptable concentrations.

Additionally, the present invention relates to the use of 4,5,6,7-tetrahydrobenzo[b]thiophene compounds of the general formula (I) as well as pharmaceutically acceptable salts thereof for the manufacture of pharmaceutical compositions for the prophylaxis and/or treatment of virally and/or bacterially induced diseases, particularly mycobacteria-induced infections, including opportunistic infections, and diseases.

The 4,5,6,7-tetrahydrobenzo[b]thiophene compounds as well as pharmaceutically acceptable salts thereof according to the present invention are effective against mycobacteria-induced infections, particularly tuberculosis, but also, e.g., leprosy and mycobacteria-induced meningitis. Mycobacteria which induce or cause these infectious diseases are members of the group comprising the tuberculous bacteria Mycobacterium tuberculosis, M. bovis, M. africanum and M. leprae as well as the non-tuberculous bacteria M. abscessus, M. avium, M. celatum, M. chelonae, M. fortuitum, M. genavense, M. gordonae, M. haemophilum, M. intracellulare, M. kansii, M. malmoense, M. marinum, M. scrofulaceum, M. simiae, M. szulgai, M. ulcerans and M. xenopi. Because of the outstanding clinical importance of tuberculosis, microbiologists have distinguished the so-called “Mycobacterium tuberculosis complex” consisting of Mycobacterium tuberculosis, M. bovis, and M. africanum from all other mycobacteria which form the group of the so-called “atypical mycobacteria” or “non-tuberculous mycobacteria (NTM)”.

The present invention also provides a method for treating mycobacteria-induced infections (including opportunistic infections) in mammals (including humans), which method comprises administering to the mammal an amount of at least one 4,5,6,7-tetrahydrobenzo[b]thiophene derivative and/or a pharmaceutically acceptable salts thereof effective to treat a mycobacteria-induced infection. Especially, the method is used for the treatment of tuberculosis, but also for other mycobacteria-induced infections like leprosy or mycobacteria-induced meningitis.

The present invention also provides pharmaceutical compositions comprising at least one 4,5,6,7-tetrahydrobenzo[b]thiophene compound as an active ingredient together with at least one pharmaceutically acceptable (i.e., non-toxic) carrier, excipient and/or diluent. The pharmaceutical compositions of the present invention can be prepared in a conventional solid or liquid carrier or diluent, and optionally with conventional pharmaceutically acceptable adjuvants, incorporating suitable dosage levels, using techniques well known in the art. The preferred preparations are adapted for oral administration. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutaneous, rectal, subcutaneous, sublingual, topical, or transdermal application, which preparations in addition to typical vehicles and/or diluents contain at least one 4,5,6,7-tetrahydrobenzo[b]thiophene compound and/or a pharmaceutical acceptable salt thereof as active ingredient.

The pharmaceutical compositions according to the present invention containing at least one 4,5,6,7-tetrahydrobenzo[b]thiophene compound as described herein and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95 weight % of the 4,5,6,7-tetrahydrobenzo[b]thiophene compound or the respective pharmaceutically active salt as active ingredient.

In addition to the 4,5,6,7-tetrahydrobenzo[b]thiophene compounds discussed above, it has also been discovered that benzo[g]quinoxaline compounds of the general formula (II) below are able to specifically inhibit growth of mycobacteria:

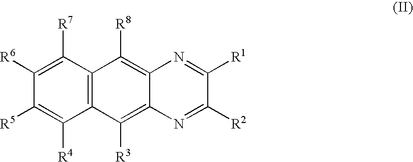

wherein:

R¹ and R² are independently of each other —CH₂)ₚ—NH—CH₂)ₙ—R⁹, —CH₂)ₛ—S—(CH₂)ₘ—R¹⁰, —CH₂)ₘ—O—(CH₂)ₚ—R¹¹, —(CH₂)ᵣ—R³, —CH═CH—R¹¹, —(CH₂)ₘ—CH(OH)—(CH₂)ₚ—R¹¹, —(CH₂)_{q}—R¹¹, —R⁹, —R¹⁰, —R¹², —R¹³,

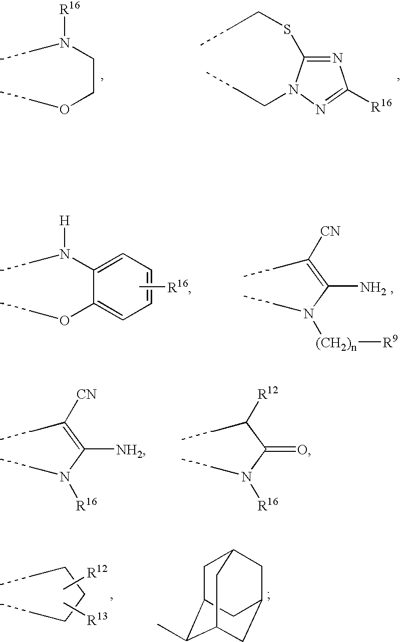

R³, R⁴, R⁵, R⁶, R⁷, and R⁸ are independently of each other —H, —F, —Cl, —Br, —I, —SO₃H, —SO₃NH₂, —(CH₂)ₛ—COOR¹⁶, —(CH₂)ₚ—COOR¹⁷, —OR¹⁶, —SR¹⁶, —NR¹⁶R¹⁷, —OOCR¹⁶, —OOCR¹⁷, —NH—CO—R¹⁶, —NH—CO—R¹⁷, —CO—NH—R¹⁶, —CO—NH—R¹⁷, —NO₂, —N₃, —CN, —OCN, —NCO, —SCN, —NCS, —CO—R¹⁶, —CO—R¹⁷, —COCN, —CONR¹⁶R¹⁷, —SOR¹⁶, —SOR¹⁷, —SO₂R¹⁶, —SO₂R¹⁷, —SO₃R¹⁶, —SO₃R¹⁷, —OCF₃;

R⁹, R¹⁰, and R¹¹ are independently of each other —CN, —NR¹⁶R¹⁷, —NHR¹⁶, —NHR¹⁷,

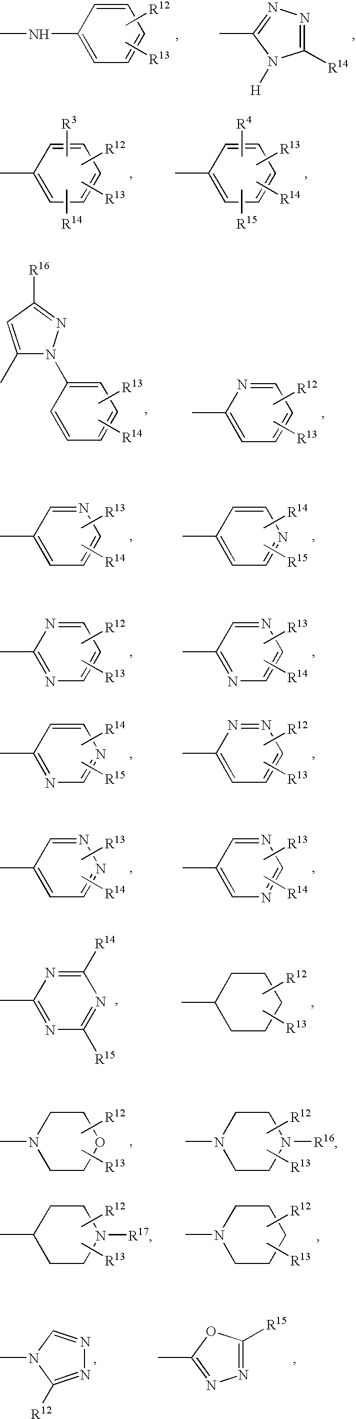

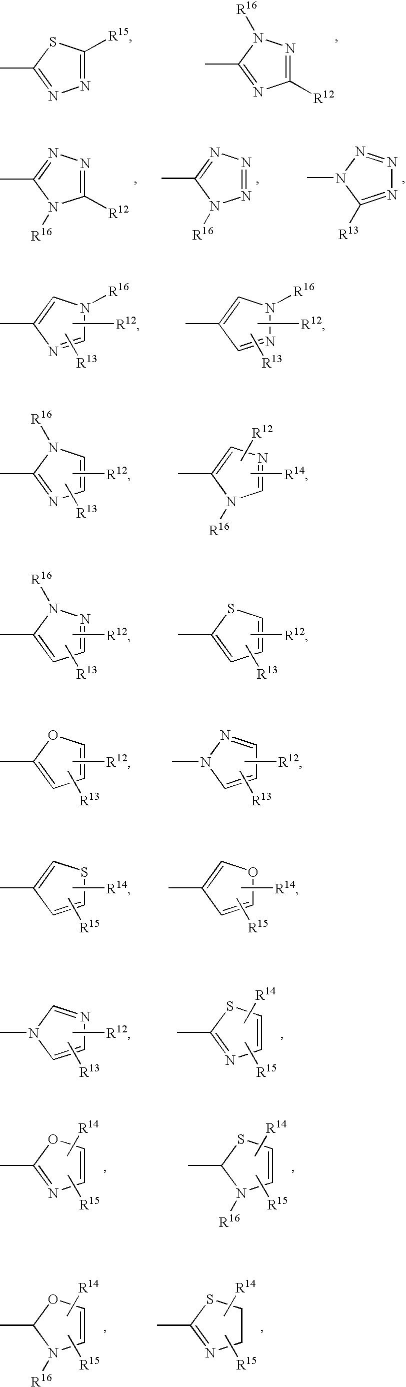

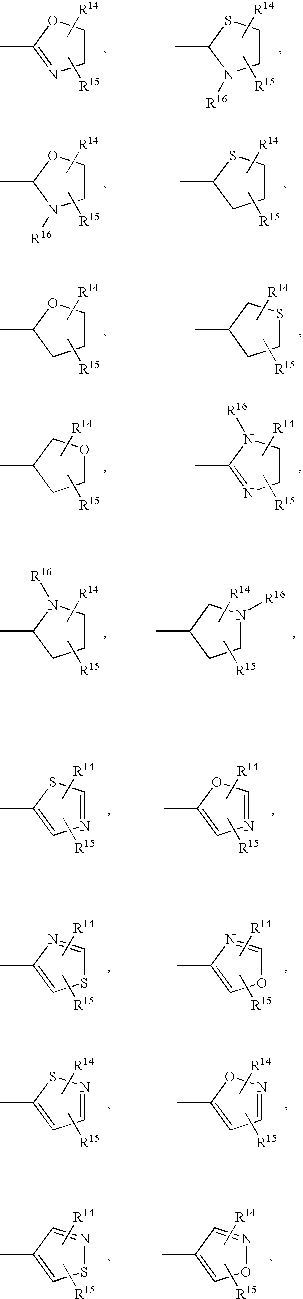

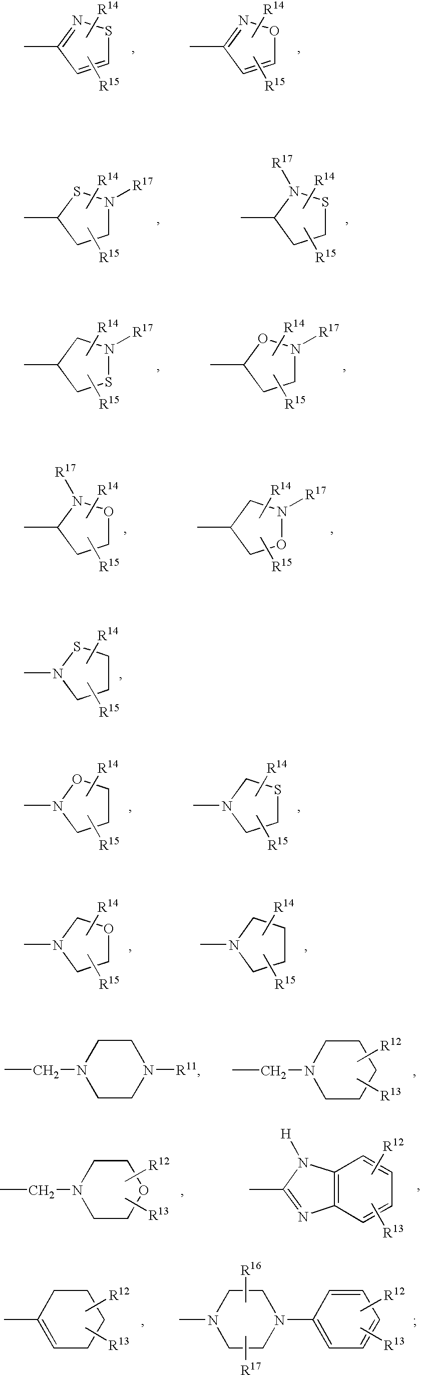

R¹², R¹³, R¹⁴, and R¹⁵ are independently of each other —R³, —R⁴, —R⁵, —R⁶, —R¹⁶, —R¹⁷, —CH(COOR¹⁶)(COOR¹⁷) —CH(CN)(COOR¹⁶),

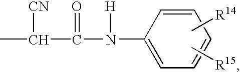

R¹⁶ and R¹⁷ are independently of each other —H, —CH₃, —C₂H₅, —C₃H₇, —CH(CH₃)₂, —C₄H₉, —C₅H₁₁, —C₆H₁₃, -cyclo-C₆H₁₁, -cyclo-C₅H₉, -cyclo-C₄H₇, -cyclo-C₃H₅, —(CH₂)ᵣ—CH(CH₃)₂, —CH(CH₃)C₂H₅, —(CH₃)₃, —CH═CH₂, —CH₂—CH═CH₂, -Ph, —CH₂Ph, —C₂H₄Ph, —CH(CN)₂, —CF₃, —CCl₃, —CBr₃, —C₂F₅, —(CH₂)ᵣ—OH, —CH₂F, —CH₂Cl, —CH₂Br, —CH₂I, —CHF₂, —CHCl₂, —CHBr₂, —(CH₂)ᵣ—SH, —C₆H₄—CH₃, —C₆H₃(CH₃)₂,

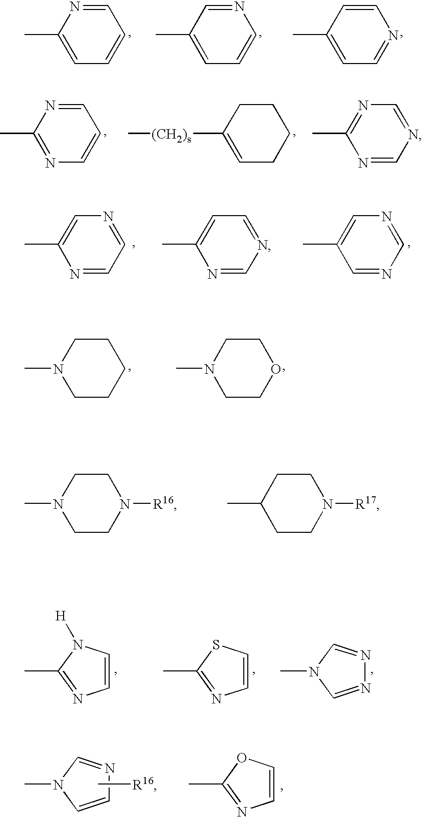

m, n, p, q, s are independently of each other integer from 0-6, r is an integer from 1-6, and the corresponding N-oxides in position 1 and/or 4 of these compounds; and the corresponding reduced forms of these compounds wherein the double bond in position 1 and/or 3 is hydrogenated; and pharmaceutically acceptable salts of these compounds.

Additional benzo[g]quinoxaline compounds useful for treating mycobacterial infections are disclosed in published international patent application WO 02/094796.

The benzo[g]quinoxaline compounds may also be used in methods of treatment and to prepared pharmaceutical compositions for the treatment of mycobacterial-induced infections, as described above.

The antiviral/antibacterial compounds of the present invention may be administered to a subject in need of treatment or prophylaxis for a virally or bacterially induced disease in an amound effective to inhibit growth or replication of the viral or bacterial pathogen. The exact effective amount of a compound useful in inhibiting viral and/or bacterial activity in the compositions and methods described herein will vary from subject to subject, depending on the age, weight and general condition of the subject, the severity of the disease or disorder that is being treated, the particular compound used, its mode of administration, and the like. Determination of an effective amount in a particular instance will be within the skill of practitioners in the art.

In the preparation of pharmaceutical compositions containing any of the active antiviral/antibacterial agents discussed above, suitable binders include starch, gelatin, natural sugars, corn sweeteners, natural and synthetic gums such as acacia, sodium alginate, carboxymethylcellulose, polyethylene glycol and waxes. Among the suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate. Suitable disintegrants, diluents, lubricants, binders, etc. are discussed in more detail below.

Moreover, the pharmaceutical compositions of the present invention may be formulated in sustained-release form to provide the rate-controlled release of any one or more of the components or active ingredients to optimize the therapeutic effect(s), e.g., antihistaminic activity, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g., nitrogen.

For preparing suppositories, a low melting wax, such as a mixture of fatty acid glycerides like cocoa butter is melted first, and the active ingredient is then dispersed homogeneously therein, e.g., by stirring. The molten, homogeneous mixture is then poured into conveniently sized molds, allowed to cool, and thereby solidified.

Also included are solid form preparations which are intended to be converted, shortly before use, to liquid form preparations for either oral or parenteral administration. Such liquid forms include solutions, suspensions, and emulsions.

The 4,5,6,7-tetrahydrobenzo[b]thiophene compounds as well as pharmaceutically acceptable salts thereof according to the present invention may also be delivered transdermally. The transdermal compositions may have the form of a cream, a lotion, an aerosol and/or an emulsion and may be included in a transdermal patch of the matrix or reservoir types that are known in the art for this purpose.

The term capsule as recited herein refers to a specific container or enclosure made, e.g., of methyl cellulose, polyvinyl alcohols, or denatured gelatins or starch for holding or containing compositions comprising the active ingredient(s). Capsules with hard shells are typically made of blends of relatively high gel strength gelatins from bones or pork skin. The capsule itself may contain small amounts of dyes, opaquing agents, plasticisers and/or preservatives.

The term tablet refers to a compressed or molded solid dosage form which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refers to powder blends containing the active ingredients and suitable diluents which can be suspended, e.g., in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75 weight %, and more preferably from about 30 to about 60 weight %.

The term disintegrants refers to materials added to the composition of a medicament to ease the breaking apart (disintegration) and release of the pharmaceutically active ingredients of the medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscaramellose, alginates such as alginic acid and sodium alginate, clays such as bentonites, and effervescent mixtures. The amount of disintegrant in the composition may range from about 2 to about 20 weight % of the composition, more preferably from about 5 to about 10 weight %.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20 weight % of the composition, preferably from about 3 to about 10 weight %, and more preferably from about 3 to about 6 weight %.

Lubricants refer to a class of substances which are added to the dosage form to enable the tablet granules, etc., after being compressed, to release from the mold or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5 weight % of the composition, preferably from about 0.5 to about 2 weight %, and more preferably from about 0.3 to about 1.5 weight % of the composition.

Glidents are materials that prevent caking of the components of the pharmaceutical composition and improve the flow characteristics of granulate so that flow is smooth and uniform. Suitable glidents include silicon dioxide and talc. The amount of glident in the composition may range from about 0.1 to about 5 weight % of the final composition, preferably from about 0.5 to about 2 weight %.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5 weight % of the composition, preferably from about 0.1 to about 1 weight %.

The following examples are included to illustrate the validation of mycobacterial protein kinases as therapeutic targets for treating mycobacterial-induced infections. The examples are for illustration and are not intended to limit the scope of the invention.

### EXAMPLES

In the following documents, background information is given with regard to the methods, micoorganisms and enzymes used according to the present invention:

Peirs et al., “A serine/threonine protein kinase from Mycobacterium tuberculosis,” Eur. J. Biochem., 244(2), 604-612 (1997);

Arruda et al., Cloning of an M. tuberculosis DNA fragment associated with entry and survival inside cells,” Science, 261, 1454-1457 (1993);

Wieles et al., “Increased intracellular survival of Mycobacterium smegmatis containing the Mycobacterium leprae thioredoxin-thioredoxin reductase gene,” Infect. Immun., 65(7), 2537-2541 (1997);

Zahrt, “Mycobacterium tuberculosis signal transduction system required for persistent infections, Proc. Natl. Acad. Sci., 98 (22), 12706-12711 (2001); and

Mundayoor et al., “Identification of genes involved in the resistance of mycobacteria to killing by macrophages,” Ann. N. Y. Acad. Sci., 730, 26-36 (1994).

### Example 1

### Novel Compounds for the Treatment of Mycobacterial Infections

Over 5000 putative kinase inhibitors were tested for their activity to inhibit the growth of M. bovis BCG, M. tuberculosis Erdmann, and E. coli XI-1 blue in vitro. As shown in Table 1, the benzo[g]quinoxaline compounds disclosed herein exert their antiproliferative effect on M. bovis BCG and M. tuberculosis Erdmann at concentrations between <<1 μM and 32 μM. In contrast, growth of E. coli XI-1 blue was not affected by benzo[g]quinoxalines at concentrations higher than 10 μM. This demonstrates that the benzo[g]quinoxaline compounds specifically inhibit growth of mycobacteria. This also suggests that kinases which are completely lacking in E.coli are involved in mycobacterial proliferation.

TABLE 1
Growth inhibition of M. bovis BCG, M. tuberculosis Erdmann and E. coli XI-1
blue by benzo[g]quinoxaline derivatives of the formula:

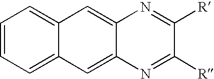

where R′ and R″ have the values listed in the table below.

| | | | | M. bovis | M. tuberculosis | E. coli |
|---|---|---|---|---|---|---|
| | | | Inhibition [%] | Inhibition | Inhibition | Inhibition |
| No. | R′ | R″ | at 1 μM | IC₅₀ [μM] | IC₅₀ [μM] | IC₅₀ [μM] |
| 1 | 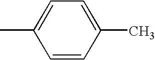 | H | 46 | 1,2 | | >>10 |
| 2 | 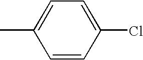 | H | 96 | <<1 | | >>10 |
| 3 | 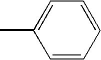 | Ph | 56 | 7,7 | | >>10 |
| 4 | 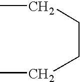 | | 21 | 12 | | >>10 |
| 5 | 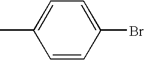 | H | 98 | <<1 | | >>10 |
| 6 | 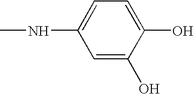 | H | 100 | <<1 | | >>10 |
| 7 | 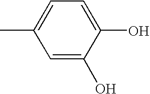 | H | 99 | 32 | | >>10 |
| 8 | 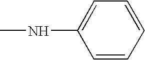 | H | 100 | <<1 | 10-20 | >>10 |
| 9 | 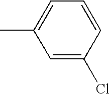 | H | 8 | 18 | | >>10 |
| 10 | 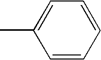 | H | 54 | 0,9 | | >>10 |
| 11 | 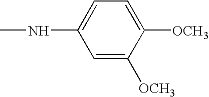 | H | 10 | | | >>10 |

In order to test whether mycobacterial kinases are inhibited by benzo[g]quinoxaline compounds, in vitro kinase assays were performed using recombinant GST-tagged Pkn A, Pkn B, Pkn D, Pkn E, Pkn F, Pkn G, Pkn H, Pkn I, Pkn J, Pkn K and Pkn L. Initial results indicated that PknB, PknG and PknH were inhibited by the inventive benzo[g]quinoxaline derivatives. Further study of these protein kinases led to the discovery of the involvement of PknG in the virulence of M. tuberculosis and to the identification of the class of 4,5,6,7-tetrahydrobenzo[b]thiophene inhibitor compounds of formula (I).

Bacterial Strains and Culture Conditions

Mycobacterium smegmatis was grown in Middlebrook 7H9 medium (supplier: Difco), supplemented with 10% ADC (Difco), 0.05% Tween-80 and 0.5% glycerol. E. coli was cultivated in LB- or TB-broth without any additional ingredients. Cloning, mutagenesis, and expression of PknG and other mycobacterial kinases was done as described by Koul et. al. (“Serine/threonine kinases, PknG and PknF of Mycobacterium tuberculosis: characterisation and localisation,” Microbiology, 147: 2307-2314 (2001)).

GST-Fusion Protein Purification

Purification of GST-fusion proteins was done as described previously by Koul et al. (“Serine/threonine kinases, PknG and PknF of Mycobacterium tuberculosis: characterisation and localisation,” Microbiology, 147: 2307-2314 (2001)). E. coli BL21 cultures containing the respective plasmids were grown overnight in TB-broth. After IPTG induction, the suspensions were incubated for another 16 hours at room temperature. The bacteria were harvested by centrifugation, resuspended in 1×PBS and lysed by sonication. After addition of Triton X-100 (1% final concentration) and subsequent clarifying of the lysates the GST-fusion proteins were purified by addition of GST-sepharose following PBS washes. The proteins were eluted with a buffer containing 50 mM glutathion, 20 mM Tris (pH 8.0), 0.1 M NaCl, 0.1 M Triton X-100 and 1 mM DTT. Thereafter, the eluates were dialyzed in 20 mM HEPES (pH 7.5) and 30% glycerol.

Determination of Protein Kinase activity

The activity of all serine/threonine protein kinases from Mycobacterium tuberculosis was determined by addition of myelin basic protein as a substrate in an in vitro kinase assay. The buffer conditions were as follows: 20 mM HEPES (pH 7.5), 20 mM MgCl₂, and 5 mM MnCl₂, for all kinases except PknG, PknI, PknJ, and PknL. These protein kinases required lower salt concentrations, namely 1 mM MgCl₂, and 1 mM MnCl₂. The optimum ATP concentration for each kinase was determined by titration of ATP in a range between 0.0033 μM and 100 μM. The inhibitor studies were performed with ATP concentrations similar to the Michaelis constant (Kₘ) for ATP. We further analyzed the role of PknG in pathogenesis of mycobacteria in a cellular infection model.

### Example 2

### Infection of macrophage cells with recombinant Mycobacterium smegmatis

Mycobacterium smegmatis, electroporated with either vector alone or mycobacterial expression vector containing PknG (wild type) or PknG-K181M (Mutant), was cultured for 2 days in Middlebrook 7H9 medium containing 0.05% Tween-80 and 0.5% glycerol. Bacteria were pelleted at 1500×g for 3 minutes by centrifugation and resuspended by vigorous agitating (Vortex) in Dulbecco's modified Eagle's medium (DMEM, GIBCO-BRL, Gaithersburg, USA) containing 5% fetal calf serum (FCS) for infecting murine macrophage cell line RAW (American Type Culture Collection, accession no. 91B-71). This yielded a bacterial supernatant consisting mostly of single mycobacterial cells as observed by acid fast staining. Under the assumption that an optical density (O.D.) of 0.1 at 650 nm equates to 10⁸ CFU/ml (see, in this respect, Wei et al., “Identification of a Mycobacterium tuberculosis Gene that Enhances Survival of M. smegmatis in Macrophages”, J. Bacteriol., 182, 377-384 (2000)), the O.D. of Mycobacterium smegmatis cell suspension was measured and diluted to 5×10⁶ CFU/ml in DMEM containing 5% FCS. Viable counts were performed on Middlebrook 7H10 medium.

The RAW cell line was maintained in DMEM medium supplemented with 10% FCS. The survival assay for recombinant Mycobacterium smegmatis was performed as described by Wei et al., cited above. RAW cells were plated in a 24 well tissue culture plate (4×10⁵ cells/well) and incubated overnight in 5% CO₂ at 37° C. The inoculum (1 ml) containing 5×10⁶ recombinant Mycobacterium smegmatis was added to achieve muliplicities of infection (moi) of 10. The plate was incubated for 2 hours at 37° C. in 5% CO₂. The infected monolayers were washed twice with warm DMEM and treated with 2% FCS containing 200 μg of amikacin/ml for 1 hour at 37° C. to kill extracellular M. smegmatis. The cells were again washed twice with warm DMEM and further incubated in DMEM containing 20 μg of amikacin. This time point was considered 0 hours of infection. The 24 hours infected monolayer was incubated with 20 μg of amikacin/ml to prevent extracellular growth of any bacteria released by premature lysis of infected RAW cells. Cells were washed twice with warm DMEM before lysis was effected by addition of a 0.1% SDS solution and vigorously pipeting several times to ensure lysis of cells and release of surviving bacteria. The lysates were diluted in 7H9 broth and plated onto 7H10 agar plates and CFU were counted after incubation at 37° C. for 4 to 5 days.

Validation of Mycobacterial Kinase as a Mycobacterial Virulence Gene

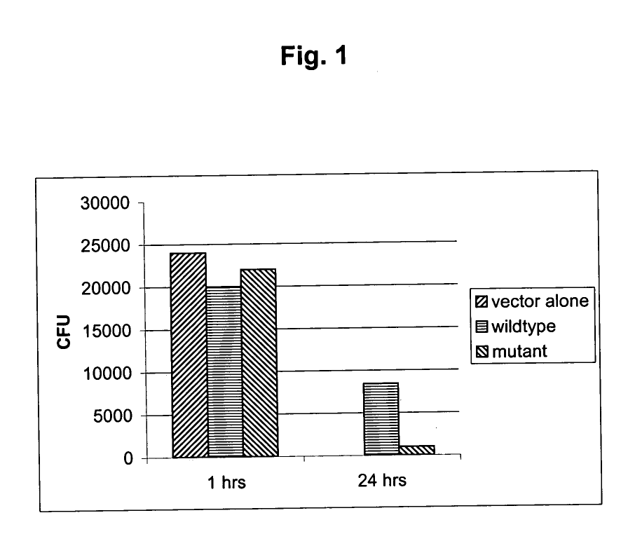

Mycobacterium smegmatis was electroporated either with wildtype or mutant kinase (which exerts no kinase activity) or vector control. A murine macrophage cell line (RAW) was infected with the various recombinant M. smegmatis expressing either pknG wild type or PknG K/M mutant or vector alone. After infection, the cells were lysed at different time points and the amount of intracellular bacteria was analysed. As can be seen from FIG. 1, after one hour postinfection the amount of bacteria recovered from macrophages infected with M. smegmatis expressing PknG wild type, PknG K/M mutant, or vector alone was the same. This shows that the recombinant M. smegmatis strains were internalized with equal efficiency. However, 24 hours postinfection the amount of M. smegmatis transformed with the vector control or the mutant kinase was substantially decreased within macrophages. This shows an efficient clearance or degradation of the the M. smegmatis expression vector alone or PknG K/M mutant by the lysosomal degradation pathway within the macrophages. In contrast, after 24 hours the amount of M. smegmatis expressing wildtype PknG which survived within cells was ten times the amount shown by cells transfected with M. smegmatis expressing the mutant kinase. This clearly demonstrates that the kinase activity of PknG increases the intracellular survival of M. smegmatis within macrophages and as such shows PknG to be an important virulence factor of mycobacteria. Consequently, the kinase is a promising target for recognizing, monitoring, and controlling therapy of various diseases.

### Example 3

### Screening for Inhibitors of PknG

A search was conducted for specific molecules inhibiting the target kinase (PknG) of Mycobacterium tuberculosis. In a kinase platform a suitable substrate was identified and an in vitro assay was adapted to high throughput screening. Subsequently, a library comprising 55,000 compounds (Discovery Partners Int'l, Allschwil, CH) using the established in vitro kinase assay was screened. Table 2 shows the half-maximal inhibition constant (IC₅₀) values of the following small synthetic molecules for inhibiting mycobacterial PknG:

Compound 237: 2-(Cyclopropanecarbonyl-amino)-4,5,6,7-tetrahydro-benzo[b]thiophene-3-carboxylic acid amide,

Compound 238: 2-[2-(4-Nitrophenyl)-acetylamino]-4,5,6,7-tetrahydro-benzo[b]thiophene-3-carboxylic acid amide,

Compound 239: 6-Methyl-2-[2-(3-nitro-[1,2,4]triazol-1-yl)-acetylamino]-4,5,6,7-tetrahydro-benzo[b]thiophene-3-carboxylic acid amide,

Compound 240: Furan-2-carboxylic acid [3-(2-hydroxy-ethylcarbamoyl)-4,5,6,7-tetrahydro-benzo[b]thiophen-2-yl]-amide,

Compound 250: 2-Acetyl-amino-4,5,6,7-tetrahydrobenzo[b]thiophene-3-carboxylic acid amide,

Compound 252: 2-(Cyclopropanecarbonyl-amino)-4,5,6,7-tetrahydro-benzo[b]thiophene-3-carboxylic acid,

Compound 255: 2-Isobutyrylamino-4,5,6,7-tetrahydrobenzo[b]thiophene-3-carboxylic acid amide,

Compound 256: 2-Isobutyrylamino-4,5,6,7-tetrahydrobenzo[b]thiophene-3-carboxylic acid ethyl ester,

Compound 266: 2-Cyclobutanecarbonyl-amino)-4,5,6,7-tetrahydro-benzo[b]thiophene-3-carboxylic acid amide,

Compound 267: 2-(2-Methyl-butyrylamino)-4,5,6,7-tetrahydrobenzo[b]thiophene-3-carboxylic acid amide,

Compound 270: 2-(Cyclopropanecarbonyl-amino)-6-methyl-4,5,6,7-tetrahydrobenzo[b]thiophene-3-carboxylic acid amide,

Compound 284: 2-Acetylamino-6-methyl-4,5,6,7-tetrahydrobenzo[b]thiophene-3-carboxylic acid amide, and

Compound 286: 2-(Cyclopropanecarbonyl-amino)-4,7-dihydro-5H-thieno[2,3-c]-thiopyran-3-carboxylic acid amide.

As is evident from Table 2, Compound 237 and Compound 270 are the most effective ones among of those tested in inhibiting the activity of serine/threonine protein kinase G of M. tuberculosis, Compound 270 having an IC₅₀ value of only 900 nM and Compound 237 having an IC₅₀ value of only 200 nM. With Compounds 238, 239, 240, 250, 252, 255, 256, 266, 267, 284, and 286 satisfactory results also were obtained, with the compounds having IC₅₀ values ranging from about 2 μM and more than 30 μM.

TABLE 2
Small molecule inhibitors of mycobacterial
protein kinase G (PknG)

| Compound No. | Inhibition of PknG (IC₅₀, [μM]) |
|---|---|
| Compound 237 | 0.2 |
| Compound 238 | ˜5 |
| Compound 239 | >30 |
| Compound 240 | 16 |
| Compound 250 | >30 |
| Compound 252 | ˜19 |
| Compound 255 | ˜3 |
| Compound 256 | >30 |
| Compound 266 | ˜3 |
| Compound 267 | ˜15 |
| Compound 270 | 0.9 |
| Compound 284 | ˜12 |
| Compound 286 | ˜2 |

### Example 4

### Survival of Mycobacterium smegmatis Transformed with Wildtype PknG within Cells in the Presence or Absence of Compound 237

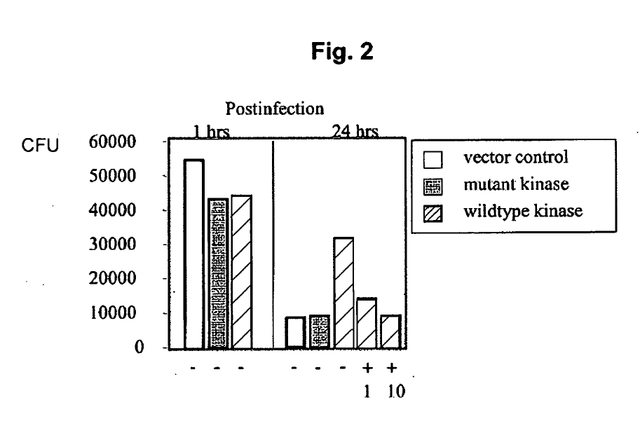

M. smegmatis was electroporated with either a wildtype or a mutant PknG expression vector construct or with and empty vector control, and then used to transfect RAW cells. RAW cells were also infected with recombinant M.smegmatis expressing wildtype PknG in the presence of different concentrations (1 μM and 10 μM) of Compound 237. After infection, the cells were lysed at different time points and the amount of intracellular bacteria was analysed. At one hour postinfection equal amounts of bacteria were recovered from the macrophages expressing either PknG wild type or mutant or vector alone (see FIG. 2). However 24 hours postinfection, as shown in FIG. 2, bacteria transformed with mutant or vector control were cleared or degraded in macrophages by up to 85%. In contrast, M.smegmatis expressing wildtype pknG was cleared only 25%, showing an increased resistance towards intracellular degradation within the macrophage cell line. In the presence of 10 μM Compound 237, however, the clearance of PknG-expressing M.smegmatis was as efficient as observed with the mutant or vector control transformants (FIG. 2). This shows that PknG kinase activity was essential for the increased intracellular survival of M. smegmatis within macrophages. Furthermore, this demonstrates that Compound 237 affected survival of M. smegmatis expressing PknG wildtype within macrophages.

### Example 5

### Secretion of PknG Outside the Bacterial Cell

In the following example we demonstrated that PknG is secreted outside the cell into the culture supernatant by mycobacterial cells.

1) PknG and ESAT (a secretory protein that acts as a positive control) were cloned into the BamH1 site of pYUB 2401. This vector contains the promoter for hsp60. An in-frame fusion with the start of hsp60 and phoA at the C-terminus were accomplished by cloning into the BamH1 site. The vector is kanamycin resistant. After cloning PknG and ESAT into pYUB2401, they were electroporated into M. smegmatis and the colonies were grown on LB plates with 40 μg of 5-bromo-4-chloro-3-indoylphosphate (BCIP) and with 20 μg of kanamycin used for screening.

PhoA fusion proteins that are exported beyond the cytoplasm are enzymatically active and capable of hydrolyzing the BCIP, the chromogenic substrate of PhoA, to produce blue colonies.

2) M. smegmatis strains containing plasmids for expressing one of the following fusions:

1) ESAT-PhoA (pYUB-ESAT)

2) PknG-PhoA (pYUB-PknG)

3) PhoA alone (pYUB2401-MocK)

were grown in 7H9 medium with kanamycin to saturation for 5-6 days and then diluted to the final optical density (O.D.) of 0.005 at 600 nm.

3) These cultures were then grown for 40 hours at 37° C. The OD₆₀₀ of each strain was measured at the start of the experiment.

4) A 0.5 ml portion of the cell culture was pelleted and resuspended in equal volume 1 M Tris (pH. 8.0).

5) Then 0.1 ml of cells was added to 1.0 ml of 2 mM p-nitrophenyl phosphate plus sodium salt in 1 M Tris (pH 8.0).

6) The reaction was incubated in the dark at 37° C. until a yellow reaction product was formed.

7) Next, 0.1 ml of 1 M K₂HP0₄ was added to terminate the reaction.

8) The bacteria were pelleted and the OD₄₂₀ of 1.0 ml of the supernatant was measured.

9) Alkaline phosphatase activity units were determined by the following formula:

$$\frac{1000 \times {OD}_{420}}{{OD}_{600} \times 0.1 ml volume of cells}$$

The negative controls were M. smegmatis cells alone and PhoA transfected M. smegmatis cells.

The above method is described in Braunstein M, Griffin T J IV, Kriakov J I, Friedman S T, Grindley N D, Jacobs W R Jr., “Identification of genes encoding exported Mycobacterium tuberculosis proteins using a Tn552′phoA in vitro transposition system,” J. Bacteriol., 182(10):2732-40 (May 2000).

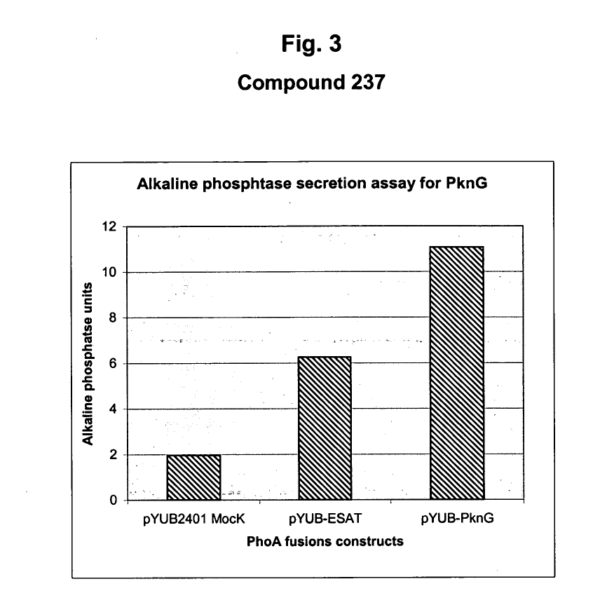

The results of the foregoing experiment show that PknG is a secretory protein that is secreted outside the mycobacterial cells. FIG. 3 shows the alkaline phosphatase secretion assay for PknG for different PhoA fusion constructs. The secreted PknG can phosphorylate host cell proteins that might be critical in survival of mycobacterium in host cells.

Based on the surprising discovery that certain mycobacterial serine/threonine protein kinases, particularly Mycobacterium tuberculosis protein kinase G (PknG), have been identified as essential components involved in the persistence and enhanced survival of pathogenic mycobacteria within a macrophage cell line, additional screening assays using such kinases (and preferably PknG) may readily be conducted to discover novel compounds useful in the treatment of mycobacterial infections, particularly Mycobacterium tuberculosis infections.

All patents, applications, and publications cited in the above text are incorporated herein by reference.

Other variations and embodiments of the invention described herein will now be apparent to those of ordinary skill in the art without departing from the scope of the invention or the spirit of the claims below.

What is claimed is:

1. A method of treating a virally or bacterially induced disease comprising administering an effective amount of an inhibitor of a mycobacterial serine/threonine protein kinase to a patient in need thereof.

2. The method according to claim 1, wherein said disease is a bacterially induced disease.

3. The method according to claim 1, wherein said disease is a mycobacterial-induced infection.

4. The method according to claim 3, wherein said mycobacterial-induced infection is tuberculosis, leprosy, or mycobacteria-induced meningitis.

5. The method according to claim 3, wherein said mycobactial-induced infection is caused by Mycobacterium tuberculosis, M. bovis, M. africanum, M. leprae, M. abscessus, M. avium, M. celatum, M. chelonae, M. fortuitum, M. genavense, M. gordonae, M. haemophilum, M. intracellulare, M. kansii, M. malmoense, M. marinum, M. scrofulaceum, M. simiae, M. szulgai, M. ulcerans and/or M. xenopi.

6. The method according to claim 5, wherein said mycobacterial-induced infection is a Mycobacterium tuberculosis infection.

7. The method according to claim 1, wherein said mycobacterial serine/threonine protein kinase is selected from the group consisting of PknG, PknB, and PknH.

8. The method according to claim 7, wherein said mycobacterial serine/threonine protein kinase is PknG.

9. The method according to claim 7, wherein said mycobacterial serine/threonine protein kinase is PknB.

10. The method according to claim 7, wherein said mycobacterial serine/threonine protein kinase is PknH.

11. The method according to claim 1 or claim 8, wherein said inhibitor is a 4,5,6,7-tetrahydrobenzo[b]thiophene compound having the following general formula (I): 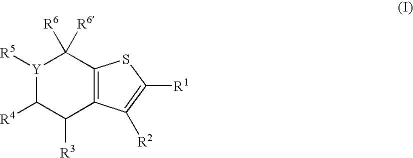
wherein
Y represents C or S;
wherein
R¹ represents R², —NH—CO—R¹⁶, —NH₂, —N═CH—R¹⁵, —N═CH—R¹⁶, —NH—CH₂—R¹⁴, —NH—CH₂—R¹⁶, —NH—SO₂—R¹⁴, —NH—CO—NH—R¹⁴, —NH—CS—NH—CH(CCl₃)—NH—CO—R¹⁶, —NH—CH(CCl₃)—NH—CO—R¹², —NH—CS—NH—R¹², —N═CH—R¹², 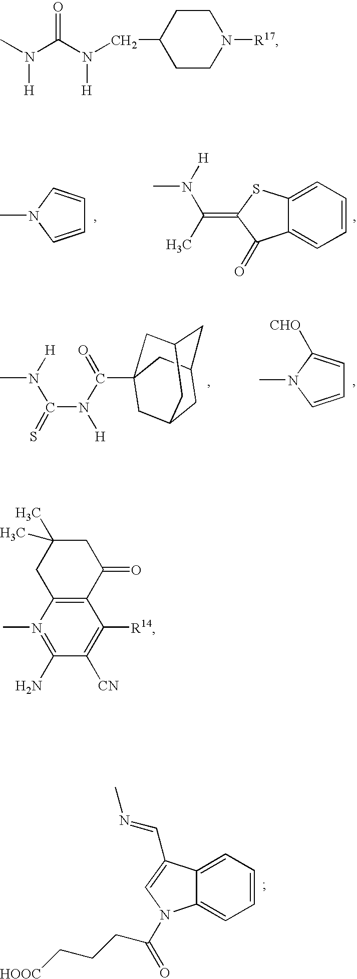
R² represents —COOR¹², —CONR¹²R^{12′}, —CONR¹²R¹⁴, —C≡N, —COCOOR¹², —COCH₂Cl, —COCONHNH₂,
R³, R⁴, R⁵ represent independently of each other —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR¹², —SO₂R¹², —SO₃R¹², —CH₂OR¹²;
in one case R⁶ represents —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR¹², —SO₂R¹², —SO₃R¹², and R^{6′} is hydrogen;
in the other case R⁶ and R^{6′} together represent a carbonyl oxygen or a oximo residue ═N—OH or ═N—OOC—R¹²;
R⁷, R⁸, R⁹, R¹⁰ represent independently of each other —R¹¹, —R¹², —OR¹², —SR¹², —NO₂, —CO—R¹², —COOR¹², —OOCR¹⁶;
R¹¹ represents —F, —Cl, —Br, —I;
R¹², R^{12′} represent independently of each other —H, —CH₃, —C(R¹¹)₃, —C₂H₅, —C₃H₇, —CH(CH₃)₂, —CH₂—CH═CH₂, —CCH₃═CH₂, —CH═C(CH₃)₂, —CH═CH—CH₃, —CH═CHC₂H₅, —CH(CH₃)C₂H₅, —(CH₂)ₙ—C≡C—R⁵, —C₄H₉, —C(CH₃)₃, -Ph, —CH₂—R¹⁵; —C₂H₄OH with n being an integer from 0-2;
R¹³ represents —CF₂CHF₂, —C₅H₁₁, —C₆H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —CH₂SPh, —CH₂R¹¹, —C₂H₄R¹¹, —C₃H₆R¹¹, —C₄H₈R¹¹, —C₂H₄Ph, —CH═CH—COOR¹², —CH₂COOR¹², —C₂H₄COOR¹², —C₃H₆COOR¹², —CH(Ph)-SPh, —C₃H₅, —CH₂CH(Ph)₂, —C₄H₇, —C₅H₉, —C(CH₃)₂CH₂R¹¹, —CH₂CH(CH₃)₂, —CH(R¹¹)Ph, —CH₂CH(CH₃)CH₂C(CH₃)₃, —CH(C₂H₅)—C₄H₉, —CH(R¹¹)₂, —CH(Ph)—C₂H₅, —CH₂C(CH₃)₃,
R¹⁴ represents 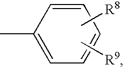
R¹⁵ represents 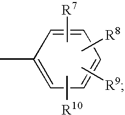
R¹⁶ is R¹², 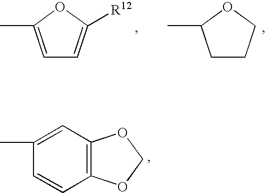
R¹³, R¹⁵, 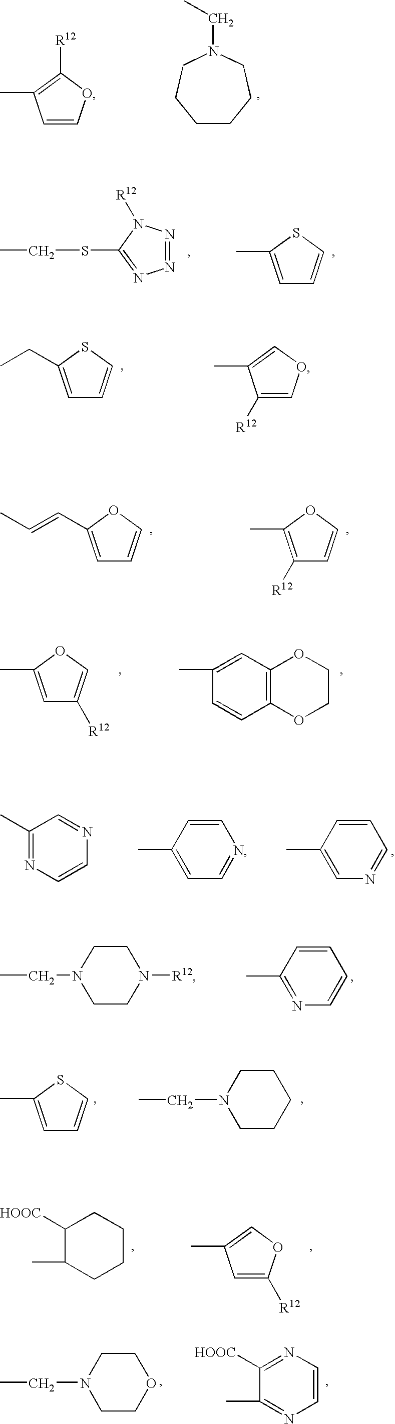 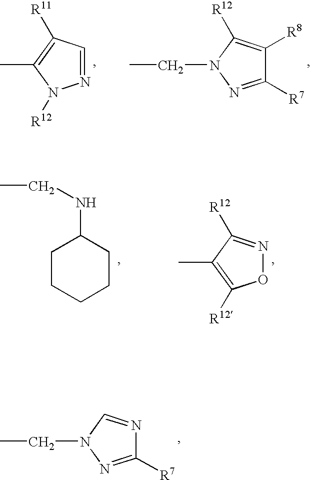
—CH₂O—R¹⁴, —CH═CH—R¹⁴, —CH₂CH(R¹⁴)—CO—R¹², —C₂H₄O—R¹⁴, —C₃H₆O—R¹⁴, —C₂H₄S—CO—R¹², —CH₂S—CO—R¹²; —COOR^{12,} 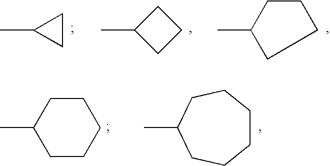
nitrobenzyl, particularly p-nitrobenzyl;
R¹⁷ represents —H, —CO—R¹², —CO—R¹³, —CO—R¹⁴, —CO—NH—R¹², —CO—NH—R¹³, —CO—NH—R¹⁴, —SO₂—R¹⁴, —CO—NH—CH₂—COO—R¹², —CO—CH₂—O—R¹⁴, —CO—CH═CH—R¹⁴, 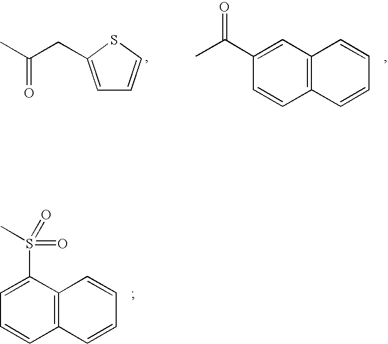
or R¹ and R² together represent a heterocyclic ring system having the following formula 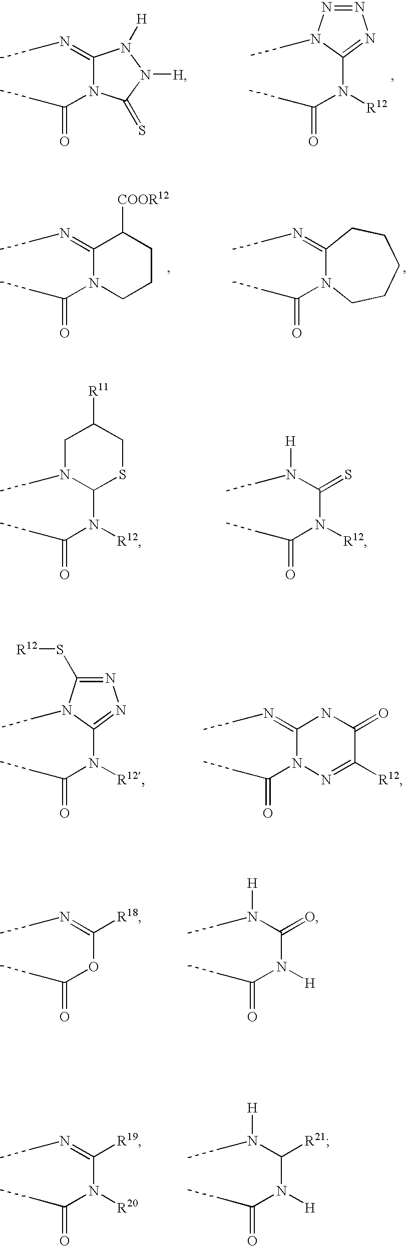
R¹⁸ represents R¹², 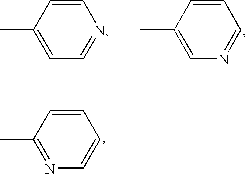
R¹⁹ represents R³, R¹⁴, —SCH₂—R³, —SCH₂—CO—R¹⁴, —SCH₂—CO—NH—R¹⁴, —SCH₂—CO—NH—CH₂—R¹², —NH—CO—CH₂—OR¹⁴, —CO—NH—N═CHR¹²(R¹⁴), 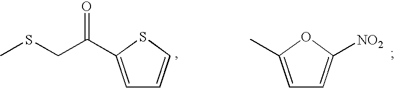
R²⁰ represents R¹², —NH—CO—R¹², —N═CH—R¹⁵;
R²¹ represents R¹⁵, 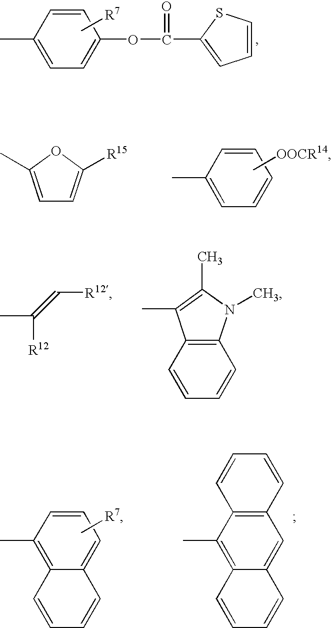
and pharmaceutically acceptable salts of these compounds.

12. The method of claim 11, wherein said 4,5,6,7-tetrahydrobenzo[b]thiophene compound is selected from the group consisting of:
(Compound 1) 2-{3-[1-(2-Chloro-acetyl)-piperidin-4-ylmethyl]-ureido}-4,5,6,7-tetrahydro-benzo[b]thiophene-3-carboxylic acid ethyl ester;
(Compound 2) 2-[3-(1-Butyryl-piperidin-4-ylmethyl)-ureido]-4,5,6,7-tetrahydro-benzo[b]thiophene-3-carboxylic acid ethyl ester;
(Compound 3) 2-[3-(1-Propanoyl-piperidin-4-ylmethyl)-ureido]-4,5,6,7-tetrahydro-benzo[b]thiophene-3-carboxylic acid ethyl ester;
(Compound 4) 2-[3-(1-Bemzoyl-piperidin-4-ylmethyl)-ureido]-4,5,6,7-tetrahydro-benzo[b]thiophene-3-carboxylic acid ethyl ester;
(Compound 5) 2-{3-[1-(2-Chloro-benzoyl-piperidin-4-ylmethyl]-ureido}-4,5,6,7-tetrahydro-benzo[b]thiophene-3-carboxylic acid ethyl ester;
(Compound 6) 2-[3-(1-Isobutyryl-piperidin-4-ylmethyl)-ureido]-4,5,6,7-tetrahydro-benzo[b]thiophene-3-carboxylic acid ethyl ester;
(Compound 7) 2-{3-[1-(4-Methyl-benzoyl-piperidin-4-ylmethyl]-ureido}-4,5,6,7-tetrahydro-benzo[b]thiophene-3-carboxylic acid ethyl ester;
(Compound 8) 2-[3-(1-Cyclohexanecarbonyl-piperidin-4-ylmethyl)-ureido]-4,5,6,7-tetrahydro-benzo[b]thiophene-3-carboxylic acid ethyl ester;
(Compound 9) 2-[3-(1-Cyclopropanecarbonyl-piperidin-4-ylmethyl)-ureido]-4,5,6,7-tetrahydro-benzo[b]thiophene-3-carboxylic acid ethyl ester;
(Compound 10) 2-[3-(1-Hexanoyl-piperidin-4-ylmethyl)-ureido]-4,5,6,7-tetrahydro-benzo[b]thiophene-3-carboxylic acid ethyl ester;
(Compound 11) 2-{3-[1-(2-Methyl-benzoyl-piperidin-4-ylmethyl]-ureido}-4,5,6,7-tetrahydro-benzo[b]thiophene-3-carboxylic acid ethyl ester;
(Compound 12) 2-{3-[1-(3-Chloro-2,2-dimethyl-propionyl)-piperidin-4-ylmethyl]-ureido}-4,5,6,7-tetrahydro-benzo[b]thiophene-3-carboxylic acid ethyl ester;
(Compound 13) 2-{3-[1-(3,5,5-Trimethyl-hexanoyl-piperidin-4-ylmethyl]-ureido}4,5,6,7-tetrahydro-benzo[b]thiophene-3-carboxylic acid ethyl ester;
(Compound 14) 2-{3-[1-(2-Ethyl-hexanoyl-piperidin-4-ylmethyl]-ureido}-4,5,6,7-tetrahydro-benzo[b]thiophene-3-carboxylic acid ethyl ester;
(Compound 15) 2-{3-[1-(2-Phenyl-butyryl-piperidin-4-ylmethyl]-ureido}-4,5,6,7-tetrahydro-benzo[b]thiophene-3-carboxylic acid ethyl ester;
(Compound 16) 2-[3-(1-Cyclopentylcarbonyl-piperidin-4-ylmethyl)-ureido]-4,5,6,7-tetrahydro-benzo[b]thiophene-3-carboxylic acid ethyl ester;
(Compound 17) 2-{3-[1-(2-Chloro-2-phenyl-acetyl-piperidin-4-ylmethyl]-ureido}-4,5,6,7-tetrahydro-benzo[b]thiophene-3-carboxylic acid ethyl ester;
(Compound 18) 2-{3-[1-(4-Butyl-benzoyl-piperidin-4-ylmethyl]-ureido}-4,5,6,7-tetrahydro-benzo[b]thiophene-3-carboxylic acid ethyl ester;
(Compound 19) 2-[3-(1-Decanoyl-piperidin-4-ylmethyl)-ureido]-4,5,6,7-tetrahydro-benzo[b]thiophene-3-carboxylic acid ethyl ester;
(Compound 20) 2-[3-(1-Heptanoyl-piperidin-4-ylmethyl)-ureido]-4,5,6,7-tetrahydro-benzo[b]thiophene-3-carboxylic acid ethyl ester;
(Compound 21) 2-[3-(1-Nonanoyl-piperidin-4-ylmethyl)-ureido]-4,5,6,7-tetrahydro-benzo[b]thiophene-3-carboxylic acid ethyl ester;
(Compound 22) 2-[3-(1-Dodecanoyl-piperidin-4-ylmethyl)-ureido]-4,5,6,7-tetrahydro-benzo[b]thiophene-3-carboxylic acid ethyl ester;
(Compound 23) 2-{3-[1-(3-Methy-butyryl)-piperidin-4-ylmethyl]-ureido}-4,5,6,7-tetrahydro-benzo[b]thiophene-3-carboxylic acid ethyl ester;
(Compound 24) 2-[3-(1-Tetradecanoyl-piperidin-4-ylmethyl)-ureido]-4,5,6,7-tetrahydro-benzo[b]thiophene-3-carboxylic acid ethyl ester;
(Compound 25) 2-[3-(1-Cyclohexylcarbamoyl-piperidin-4-ylmethyl)-ureido]-4,5,6,7-tetrahydro-benzo[b]thiophene-3-carboxylic acid ethyl ester;
(Compound 26) 2-[3-(1-Phenylcarbamoyl-piperidin-4-ylmethyl)-ureido]-4,5,6,7-tetrahydro-benzo[b]thiophene-3-carboxylic acid ethyl ester;
(Compound 27) 2-[3-(1-Benzylcarbamoyl-piperidin-4-ylmethyl)-ureido]-4,5,6,7-tetrahydro-benzo[b]thiophene-3-carboxylic acid ethyl ester;
(Compound 28) 2-{3-[1-(4-Ethoxycarbonyl-phenylcarbamoyl)-piperidin-4-ylmethyl]-ureido}-4,5,6,7-tetrahydro-benzo[b]thiophene-3-carboxylic acid ethyl ester;
(Compound 29) 2-{3-[1-(3-Bromo-phenylcarbamoyl)-piperidin-4-ylmethyl]-ureido}-4,5,6,7-tetrahydro-benzo[b]thiophene-3-carboxylic acid ethyl ester;
(Compound 30) 2-{3-[1-(2-Methoxy-phenylcarbamoyl)-piperidin-4-ylmethyl]-ureido}-4,5,6,7-tetrahydro-benzo[b]thiophene-3-carboxylic acid ethyl ester;
(Compound 31) 2-{3-[1-(2-Methyl-phenylcarbamoyl)-piperidin-4-ylmethyl]-ureido}-4,5,6,7-tetrahydro-benzo[b]thiophene-3-carboxylic acid ethyl ester;
(Compound 32) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;
(Compound 33) 2-{3-[1-(4-Fluoro-benzenesulfonyl)-piperidin-4-ylmethyl]-ureido}-4,5,6,7-tetrahydro-benzo[b]thiophene-3-carboxylic acid ethyl ester;
(Compound 34) 2-{3-[1-(4-Methyl-benzenesulfonyl)-piperidin-4-ylmethyl]-ureido}-4,5,6,7-tetrahydro-benzo[b]thiophene-3-carboxylic acid ethyl ester;
(Compound 35) 2-{3-[1-Naphtalene-1-sulfonyl)-piperidin-4-ylmethyl]-ureido}-4,5,6,7-tetrahydro-benzo[b]thiophene-3-carboxylic acid ethyl ester;
(Compound 36) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;
(Compound 37) 2-{3-[1-(2,5-Dichloro-benzenesulfonyl)-piperidin-4-ylmethyl]-ureido}-4,5,6,7-tetrahydro-benzo[b]thiophene-3-carboxylic acid ethyl ester;
(Compound 38) 2-{3-[1-(2,2-Dichloro-acetyl)-piperidin-4-ylmethyl]-ureido}-4,5,6,7-tetrahydro-benzo[b]thiophene-3-carboxylic acid ethyl ester;
(Compound 39) 2-{3-[1-(3,3-Dimethy-butyryl)-piperidin-4-ylmethyl]-ureido}-4,5,6,7-tetrahydro-benzo[b]thiophene-3-carboxylic acid ethyl ester;
(Compound 40) 2-{3-[1-(Ethoxycarbonylmethyl-carbamoyl)-piperidin-4-ylmethyl]-ureido}-4,5,6,7-tetrahydro-benzo[b]thiophene-3-carboxylic acid ethyl ester;
(Compound 41) 2-{3-[1-(3-Methoxy-phenylcarbamoyl)-piperidin-4-ylmethyl]-ureido}-4,5,6,7-tetrahydro-benzo[b]thiophene-3-carboxylic acid ethyl ester;
(Compound 42) 2-{3-[1-(3,5-Bis-trifluoromethyl-benzoyl)-piperidin-4-ylmethyl]-ureido}-4,5,6,7-tetrahydro-benzo[b]thiophene-3-carboxylic acid ethyl ester;
(Compound 43) 2-[3-(1-Phenylacetyl-piperidin-4-ylmethyl]-ureido]4,5,6,7-tetrahydro-benzo[b]thiophene-3-carboxylic acid ethyl ester;
(Compound 44) 2-{3-[1-(3,4-Dichloro-benzoyl-piperidin-4-ylmethyl]-ureido}-4,5,6,7-tetrahydro-benzo[b]thiophene-3-carboxylic acid ethyl ester;
(Compound 45) 2-{3-[1-(2,4,6-Trimethyl-benzenesulfonyl)-piperidin-4-ylmethyl]-ureido}-4,5,6,7-tetrahydro-benzo[b]thiophene-3-carboxylic acid ethyl ester;
(Compound 46) 2-{3-[1-(4-Methoxy-benzenesulfonyl)-piperidin-4-ylmethyl]-ureido}-4,5,6,7-tetrahydro-benzo[b]thiophene-3-carboxylic acid ethyl ester;
(Compound 47) 2-{3-[1-(Naphtalene-2-sulfonyl)-piperidin-4-ylmethyl]-ureido}-4,5,6,7-tetrahydro-benzo[b]thiophene-3-carboxylic acid ethyl ester;
(Compound 48) 2-(3-Piperidin-4-ylmethyl-ureido)-4,5,6,7-tetrahydro-benzo[b]thiophene-3-carboxylic acid ethyl ester;
(Compound 49) 2-(3-{1-[2-(4-Chloro-phenoxy)-acetyl]-piperidin-4-ylmethyl}-ureido)-4,5,6,7-tetrahydro-benzo[b]thiophene-3-carboxylic acid ethyl ester;
(Compound 50) 2-(3-{1-[3-(4-Nitro-phenoyl)-acryloyl]-piperidin-4-ylmethyl}-ureido)-4,5,6,7-tetrahydro-benzo[b]thiophene-3-carboxylic acid ethyl ester;
(Compound 51) 2-[3-(1-Octanoyl-piperidin-4-ylmethyl)-ureido]-4,5,6,7-tetrahydro-benzo[b]thiophene-3-carboxylic acid ethyl ester;
(Compound 52) 2-[3-(1-Benzenesulfonyl-piperidin-4-ylmethyl)-ureido]-4,5,6,7-tetrahydro-benzo[b]thiophene-3-carboxylic acid ethyl ester;
(Compound 53) 2-[3-(1-Ethylylcarbamoyl-piperidin-4-ylmethyl)-ureido]-4,5,6,7-tetrahydro-benzo[b]thiophene-3-carboxylic acid ethyl ester;
(Compound 54) 2-{3-[1-(3-Fluoro-benzoyl-piperidin-4-ylmethyl]-ureido}-4,5,6,7-tetrahydro-benzo[b]thiophene-3-carboxylic acid ethyl ester;
(Compound 55) 2-{3-[1-(4-Chloro-phenylcarbamoyl)-piperidin-4-ylmethyl]-ureido}-4,5,6,7-tetrahydro-benzo[b]thiophene-3-carboxylic acid ethyl ester;
(Compound 56) 4-[3-(3-ethoxycarbonyl-4,5,6,7-tetrahydro-benzo[b]thiophene-2-yl)-ureidomethyl]-piperidne-1-carboxylic acid phenyl ester;
(Compound 57) 2-{3-[1-(3-Trifluoromethyl-benzoyl-piperidin-4-ylmethyl]-ureido}-4,5,6,7-tetrahydro-benzo[b]thiophene-3-carboxylic acid ethyl ester;
(Compound 58) 2-{3-[1-(3-Bromo-benzoyl-piperidin-4-ylmethyl]-ureido}-4,5,6,7-tetrahydro-benzo[b]thiophene-3-carboxylic acid ethyl ester;
(Compound 59) 2-{3-[1-(2,4-Dichloro-phenylcarbamoyl)-piperidin-4-ylmethyl]-ureido}-4,5,6,7-tetrahydro-benzo[b]thiophene-3-carboxylic acid ethyl ester;
(Compound 60) 2-{3-[1-(2-Trifluoromethyl-phenylcarbamoyl)-piperidin-4-ylmethyl]-ureido}-4,5,6,7-tetrahydro-benzo[b]thiophene-3-carboxylic acid ethyl ester;
(Compound 61) 2-{3-[1-(4-tert-Butyl-benzoyl-piperidin-4-ylmethyl]-ureido}-4,5,6,7-tetrahydro-benzo[b]thiophene-3-carboxylic acid ethyl ester;
(Compound 62) 2-{3-[1-(3-Nitro-benzoyl-piperidin-4-ylmethyl]-ureido}-4,5,6,7-tetrahydro-benzo[b]thiophene-3-carboxylic acid ethyl ester;
(Compound 63) 2-[3-(1-Cyclobutanecarbonyl-piperidin-4-ylmethyl)-ureido]-4,5,6,7-tetrahydro-benzo[b]thiophene-3-carboxylic acid ethyl ester;
(Compound 64) 2-{3-[1-(2-Thiophen-2-ylacetyl)-piperidin-4-ylmethyl]-ureido}-4,5,6,7-tetrahydro-benzo[b]thiophene-3-carboxylic acid ethyl ester;
(Compound 65) 2-{3-[1-(Naphthalene-2-carbonyl)-piperidin-4-ylmethyl]-ureido}-4,5,6,7-tetrahydro-benzo[b]thiophene-3-carboxylic acid ethyl ester;
(Compound 66) 5,6,7,8-Tetrahydro-1H-benzol[4,5]thieno[2,3-d]pyrimidine-2,4-dione;
(Compound 67) 4-Oxo-3,4,5,6,7,8-hexahydro-benzo[4,5]thieno[2,3-d]pyrimidine-2-carboxylic acid ethyl ester;
(Compound 68) 2-Acetylamino-4,5,6,7-tetrahydro-benzo[b]thiophene-3-carboxylic acid ethyl ester;
(Compound 69) 2-Amino-4,5,6,7-tetrahydro-benzo[b]thiophene-3-carboxylic acid ethyl ester;
(Compound 70) 2-Amino-4,5,6,7-tetrahydro-benzo[b]thiophene-3-carboxylicacid amide;
(Compound 71) 2-Acetylamino-4,5,6,7-tetrahydro-benzo[b]thiophene-3-carboxylic acid;
(Compound 72) 2-(Toluene-4-sulfonylamino)-4,5,6,7-tetrahydro-benzo[b]thiophene-3-carboxylic acid amide;
(Compound 73) 7-Methyl-1,3,4,9-tetrahydro-2H-11-thia-5a,6,9,10-tetraaza-benzo[b]fluorene-5,8-dione;
(Compound 74) 7-Phenyl-1,3,4,9-tetrahydro-2H-11-thia-5a,6,9,10-tetraaza-benzo[b]fluorene-5,8-dione;
(Compound 75) 2-(5-Nitro-furan-2-yl)-2,3,5,6,7,8-hexahydro-1H-benzo[4,5]thieno[2,3-d]pyrimidin-4-one;
(Compound 76) 2-[(5-Bromo-2-hydroxy-benzylidene)-amino]-4,5,6,7-tetrahydro-benzo[b]thiophene-3-carboxylic acid amide;
(Compound 77) 2-[(4,5-Dibromo-2-hydroxy-benzylidene)-amino]-4,5,6,7-tetrahydro-benzo[b]thiophene-3-carboxylic acid ethyl ester;
(Compound 78) 2-[(2-Chloro-benzylidene)-amino]-4,5,6,7-tetrahydro-benzo[b]thiophene-3-carboxylic acid amide;
(Compound 79) 2-[1-(3-Oxo-3H-benzo[b]thiophen-2-ylidene)-ethylamino]-4,5,6,7-tetrahydro-benzo[b]thiophene-3-carboxylic ethyl ester;
(Compound 80) 2-Heptanoylamino-4,5,6,7-tetrahydro-benzo[b]thiophene-3-carboxylic acid ethyl ester;
(Compound 81) 2-(3-Bromo-benzoylamino)-4,5,6,7-tetrahydro-benzo[b]thiophene-3-carboxylic acid ethyl ester;
(Compound 82) 2-Ethyl-5,6,7,8-tetrahydro-3-oxa-9-thia-1-aza-fluoren-4-one;
(Compound 83) Furan-2-carboxylic acid [3-(4-methoxy-phenylcarbamoyl)-4,5,6,7-tetrahydro-benzo[b]thiophen-2-yl]-amide;
(Compound 84) 2-Pyridin-3-yl-5,6,7,8-tetrahydro-3-oxa-9-thia-1-aza-fluoren-4-one;
(Compound 85) 2-Propionylamino-4,5,6,7-tetrahydro-benzo[b]thiophene-3-carboxylic phenylamide;
(Compound 86) 1-Allylsulfanyl-4-phenyl-6,7,8,9-tetrahydro-4H-10-thia-2,3,4,10b-tetraaza-cyclopenta[a]fluoren-5-one;
(Compound 87) 2-[2-(1-Phenyl-1H-tetrazol-5-ylsulfanyl)-acetylamino]-4,5,6,7-tetrahydro-benzo[b]thiophene-3-carboxylic acid ethyl ester;
(Compound 88) 2-[3-(4-Methoxy-phenyl)-3-(2,2,2-trifluoro-acetylamino)-propionyl-amino]-4,5,6,7-tetrahydro-benzo[b]thiophene-3-carboxylic acid amide;
(Compound 89) 2-(3-Chloro-benzyolamino)-4,5,6,7-tetrahydro-benzo[b]thiophene-3-carboxylic acid ethyl ester;
(Compound 90) 2-{[1-(4-Carboxy-butyryl)-1H-indol-3-ylmethylene]-amino}-4,5,6,7-tetrahydro-benzo[b]thiophene-3-carboxylic acid ethyl ester;
(Compound 91) 2-(7-Ethyl-4-oxo-3-phenyl-3,4,5,6,7,8-hexahydro-benzo)[4,5]thieno[2,3-d]pyrimidin-2-ylsulfanyl)-N-(2-isopropoxy-phenyl)-acetamide;
(Compound 92) N,N-Diethyl-2-(7-ethyl-4-oxo-3-phenyl-3,4,5,6,7,8-hexahydro-benzo)[4,5]thieno[2,3-d]pyrimidin-2-ylsulfanyl)-acetamide;
(Compound 93) 3-[(4-Hydroxy-3-methoxy-benzylidene)-amino]-2-methyl-5,6,7,8-tetrahydro-3H-benzo[4,5]thieno[2,3-d]pyrimidin-4-one;
(Compound 94) 6-Ethyl-2-(3-phenyl-thioureido)-4,5,6,7-tetrahydro-benzo[b]thiophene-3-carboxylic acid ethyl ester;
(Compound 95) 2-[3-(Adamantane-1-carbonyl)-thioureido]-4,5,6,7-tetrahydro-benzo[b]thiophene-3-carboxylic acid ethyl ester;
(Compound 96) 2-[(3,5-Dibromo-2,4-dihydroxy-benzylidene)-amino]-4,5,6,7-tetrahydro-benzo[b]thiophene-3-carboxylic acid ethyl ester;
(Compound 97) Benzoic acid 2-ethoxy-4-(4-oxo-1,2,3,4,4a,5,6,7,8,9a-decahydro-benzo[4,5]thieno[2,3-d]pyrimidin-2-yl)-phenylester;
(Compound 98) 2-(1-Acetylamino-2,2,2-trichloro-ethylamino)-4,5,6,7-tetrahydro-benzo[b]thiophene-3-carboxylic acid ethyl ester;
(Compound 99) 2-(3,5-Di-tert-butyl-4-hydroxy-phenyl)-2,3,5,6,7,8-hexahydro-1H-benzo[4,5]thieno[2,3-d]pyrimidin-4-one;
(Compound 100) 4-Oxo-3,4,5,6,7,8-hexahydro-benzo[4,5]thieno[2,3-d]pyrimidine-2-carboxylic acid [1-(3-nitro-phenyl)-ethylidene]-hydrazide;
(Compound 101) 2-[(Pyridine-4-carbonyl)-amino]-4,5,6,7-tetrahydro-benzo[b]thio-phene-3-carboxylic acid ethyl ester;
(Compound 102) 2-(2,2,3,3-Tetrafluro-propionylamino)-4,5,6,7-tetrahydro-benzo[b]thiophene-3-carboxylic acid amide;
(Compound 103) 2-[3-(1-Acetylamino-2,2,2-trichloro-ethyl)-thioureido]-4,5,6,7-tetrahydro-benzo[b]thiophene-3-carboxylic acid ethyl ester;
(Compound 104) 2-[3-(2,2,2-Trichloro-1-propionylamino-ethyl)-thioureido]-4,5,6,7-tetrahydro-benzo[b]thiophene-3-carboxylic acid amide;
(Compound 105) 2-(3-Chloro-phenyl)-2,3,5,6,7,8-hexahydro-1H-benzo[4,5]thieno[2,3-d]pyrimidin-4-one;
(Compound 106) 2-(2-Chloro-phenyl)-2,3,5,6,7,8-hexahydro-1H-benzo[4,5]thieno[2,3-d]pyrimidin-4-one;
(Compound 107) 2-Anthracene-9-yl-2,3,5,6,7,8-hexahydro-1H-benzo[4,5]thieno[2,3-d]pyrimidin-4-one;
(Compound 108) 2-(3,5-Dibromo-2-methoxy-phenyl)-2,3,5,6,7,8-hexahydro-1H-benzo[4,5]thieno[2,3-d]pyrimidin-4-one;
(Compound 109) 2-[5-(4-Bromo-phenyl)-furan-2-y]-2,3,4a,5,6,7,8,9a-octahydro-1H-benzo[4,5]thieno[2,3-d]pyrimidin-4-one;
(Compound 110) 2-(2,4-Dichlorophenoxy)-N-(4-oxo-2-propyl-5,6,7,8-tetrahydro-4H-benzo[4,5]thieno[2,3-d]pyrimidin-3-yl)-acetamide;
(Compound 111) 2-(3,4-Dimethoxy-benzoylamino)-4,5,6,7-tetrahydro-benzo[b]thio-phene-3-carboxylic acid ethyl ester;
(Compound 112) 2-[3-(2-Chloro-phenyl)-acryloylamino]-4,5,6,7-tetrahydro-benzo[b]thiophene-3-carboxylic acid ethyl ester;
(Compound 113) 3-(3-Ethoxycarbonyl-4,5,6,7-tetrahydro-benzo[b]thiophene-2-ylcarb-amoyl)-pyrazine-2-carboxylic acid;
(Compound 114) 2-(3-{2,2,2-Trichloro-1-[(furan-2-carbonyl)-amino]-ethyl}-thioureido)-4,5,6,7-tetrahydro-benzo[b]thiophene-3-carboxylic acid ethyl ester;
(Compound 115) 2-(1-Methyl-2-phenyl-vinyl)-2,3,4a,5,6,7,8,9a-octahydro-1H-benzo[4,5]thieno[2,3-d]pyrimidin-4-one;
(Compound 116) 2-(2-Methoxy-naphtalen-1-yl)-2,3,4a,5,6,7,8,9a-octahydro-1H-benzo[4,5]thieno[2,3-d]pyrimidin-4-one;
(Compound 117) 2-(1,2-Dimethyl-1H-indol-3-yl)-2,3,4a,5,6,7,8,9a-octahydro-1H-benzo[4,5]thieno[2,3-d]pyrimidin-4-one;
(Compound 118) 2-(Cyclohexanecarbonyl-amino)-6-methyl-4,5,6,7-tetrahydro-benzo[b]thiophene-3-carboxylic acid ethyl ester;
(Compound 119) 3-Bromo-benzoic acid 3-(4-oxo-1,2,3,4,5,6,7,8-octahydro-benzo[4,5]thieno[2,3-d]pyrimidin-2-yl)-phenyl ester;
(Compound 120) 3-Thioxo-2,3,5,6,7,8-hexahydro-1H-9-thia-1,2,3a,10-tetraaza-cyclopenta[b]fluorene-4-one;
(Compound 121) 2-(2,3-Dibromo-5-ethoxy-4-hydroxy-phenyl)-2,3,5,6,7,8-hexahydro-1H-benzo[4,5]thieno[2,3-d]pyrimidin-4-one;
(Compound 122) 2-[2-(4-Chloro-phenoxy)-acetylamino]-6-methyl-4,5,6,7-tetrahydro-benzo[b]thiophene-3-carboxylic acid ethyl ester;
(Compound 123) 4-Phenyl-5a,6,7,8,9,10a-hexahydro-4H-10-thia-1,2,3,4,10b-pentaaza-cyclopenta[a]fluoren-5-one;
(Compound 124) 6-Methyl-2-[(thiophene-2-carbonyl)-amino]-4,5,6,7-tetrahydro-benzo[b]thiophene-3-carboxylic acid amide;
(Compound 125) 3-Allyl-2-(2-oxo-2-thiophen-2-yl-ethylsulfanyl)-5,6,7,8-tetrahydro-3H-benzo[4,5]thieno[2,3-d]pyrimidin-4-one;
(Compound 126) 2-[(2-Chloro-4-nitro-benzylidene)-amino]-4,5,6,7-tetrahydro-benzo[b]thiophene-3-carboxylic acid ethyl ester;
(Compound 127) 2-(2-Methyl-3,5-dinitro-benzoylamino)-4,5,6,7-tetrahydro-benzo[b]thiophene-3-carboxylic acid ethyl ester;
(Compound 128) 2-(4-Acetyl-benzoylamino)-4,5,6,7-tetrahydro-benzo[b]thiophene-3-carboxylic acid ethyl ester;
(Compound 129) 2-Benzoylamino-7-hydroxyimino-4,5,6,7-tetrahydro-benzo[b]thio-phene-3-carboxylic acid ethyl ester;
(Compound 130) 2-Formylamino-4,5,6,7-tetrahydro-benzo[b]thiophene-3-carboxylic acid ethyl ester;
(Compound 131) 5-Oxo-1,2,3,4,6,7,8,9-octahydro-5H-11-thia-5a,10-diaza-benzo[b]fluo-rene-9-carboxylic acid ethyl ester;
(Compound 132) 2-Benzoylamino-7-oxo-4,5,6,7-tetrahydro-benzo[b]thio-phene-3-carboxylic acid ethyl ester;
(Compound 133) 2-(2-Phenylsulfanyl-acetylamino)-4,5,6,7-tetrahydro-benzo[b]thio-phene-3-carboxylic acid ethyl ester;
(Compound 134) 2-(4-Nitro-benzylamino)-4,5,6,7-tetrahydro-benzo[b]thiophene-3-carboxylic acid ethyl ester;
(Compound 135) 2-[2-(4-Ethyl-piperazin-1-yl)-acetylamino]-7-oxo-4,5,6,7-tetrahydro-benzo[b]thiophene-3-carboxylic acid ethyl ester;
(Compound 136) 2-Phenylsulfanyl-3,4,7,8,9,10-hexahydro-2H,6H-12-thia-5,11-diaza-cyclohepta[b]fluorene-1,5-dione;
(Compound 137) 7-Hydroxy-2-(4-nitro-benzoylamino)-4,5,6,7-tetrahydro-benzo[b]thio-phene-3-carboxylic acid ethyl ester;
(Compound 138) 2-(2-Cyclohexylamino-acetylamino)-7-oxo-4,5,6,7-tetrahydro-benzo[b]thiophene-3-carboxylic acid ethyl ester;
(Compound 139) 2-(2-Azepan-1-yl-acetylamino)-7-oxo-4,5,6,7-tetrahydro-benzo[b]thio-phene-3-carboxylic acid ethyl ester;
(Compound 140) 7-Hydroxyimino-2-pentanoylamino-4,5,6,7-tetrahydro-benzo[b]thio-phene-3-carboxylic acid ethyl ester;
(Compound 141) 2-(2-Morpholin-4-yl-acetylamino)-7-oxo-4,5,6,7-tetrahydro-benzo[b]thiophene-3-carboxylic acid ethyl ester;
(Compound 142) 2-Benzoyloxyimion-4,5,7,8,9,10,11-heptahydro-3H-1-thia-6a,12-diaza-cyclohepta[b]fluorene-6-one;
(Compound 143) 2-(3-Acetylsulfanyl-propionylamino)-4,5,6,7-tetrahydro-benzo[b]thio-phene-3-carboxylic acid ethyl ester;
(Compound 144) 2-[3-(3,4-Dichloro-phenyl-ureido]-4,5,6,7-tetrahydro-benzo[b]thio-phene-3-carboxylic acid amide;
(Compound 145) 2-(2-Chloro-5-iodo-benzoylamino)-4,5,6,7-tetrahydro-benzo[b]thio-phene-3-carboxylic acid ethyl ester;
(Compound 146) 2-[(5-Benzyl-2-hydroxy-3-nitro-benzylidene)-amino]-4,5,6,7-tetrahydro-benzo[b]thiophene-3-carboxylic acid ethyl ester;
(Compound 147) 2-[(Pyridine-3-carbonyl)-amino]-4,5,6,7-tetrahydro-benzo[b]thiophene-3-carboxylic acid ethyl ester;
(Compound 148) 2-(3-Thiophen-2-yl-propionylamino)-4,5,6,7-tetrahydro-benzo[b]thiophene-3-carboxylic acid ethyl ester;
(Compound 149) 6-Methyl-2-(3-methyl-4-nitro-benzoylamino)-4,5,6,7-tetrahydro-benzo[b]thiophene-3-carboxylic acid ethyl ester;
(Compound 150) 2-Isopropyl-5,6,7,8-tetrahydro-3-oxa-9-thia-1-aza-fluor-4-one;
(Compound 151) 2-(2-Piperidin-1-yl-acetylamino)-4,5,6,7-tetrahydro-benzo[b]thio-phene-3-carboxylic acid amide;
(Compound 152) 3-Allyl-2-prop-2-ynylsulfanyl-5,6,7,8-tetrahydro-3H-benzo[4,5]thieno[2,3-d]pyrimidin-4-one;
(Compound 153) 2-(Trifluoromethyl)-5,6,7,8-tetrahydro-3H-benzo[4,5]thieno[2,3-d]pyrimidin-4-one;
(Compound 154) 2-[2-(4-Methyl-piperazin-1-yl)-acetylamino]-4,5,6,7-tetrahydro-benzo[b]thiophene-3-carboxylic acid amide;
(Compound 155) Furan-2-carboxylic acid-(3-phenylcarbamoyl-4,5,6,7-tetrahydro-benzo[b]thiophen-2-yl)-amide;
(Compound 156) 3-Phenyl-5,6,7,8-tetrahydro-3H-benzo[4,5]thieno[2,3-d]pyrimidin-4-one;
(Compound 157) 2-(4-Oxo-3-phenyl-3,4,5,6,7,8-hexahydro-benzo[4,5]thieno[2,3-d]pyrimidin-2-ylsulfanyl)-N-phenethyl-acetamide;
(Compound 158) 2-(3-Benzoylsulfanyl-acetylamino)-4,5,6,7-tetrahydro-benzo[b]-thiophene-3-carboxylic acid ethyl ester;
(Compound 159) 2-(5-Hydroxy-2-nitro-phenyl)-2,3,5,6,7,8-hexahydro-1H-benzo[4,5]thieno[2,3-d]pyrimidin-4-one;
(Compound 160) 2-[2-(2-Benzoylamino-2-carboxy-ethylsulfanyl)-acetylamino]-4,5,6,7-tetrahydro-benzo[b]thiophene-3-carboxylic acid ethyl ester;
(Compound 161) 2-[(5-Bromoo-2-hydroxy-benzylidene)-amino]-4,5,6,7-tetrahydro-benzo[b]thiophene-3-carboxylic acid ethyl ester;
(Compound 162) 3-[(2,4-Dihydroxy-benzylidene)-amino]-2-methyl-5,6,7,8-tetrahydro-3H-benzo[4,5]thieno[2,3-d]pyrimidin-4-one;
(Compound 163) N-(4-Oxo-2-pentyl-5-pentyl-5,6,7,8-tetrahydro-4H-benzo[4,5]thieno[2,3-d]pyrimidin-3-yl)-2-phenyl-acetamide;
(Compound 164) 2-[(2,3-Dihydro-benzo[1,4]dioxine-5-carbonyl)-amino]-4,5,6,7-tetrahydro-benzo[b]thiophene-3-carboxylic acid ethyl ester;
(Compound 165) 2-[(3,5-Dichloro-4-methoxy-benzylidene)-amino]-4,5,6,7-tetrahydro-benzo[b]thiophene-3-carboxylic acid ethyl ester;
(Compound 166) 2-Iodo-5-phenyl-2,3,4a,5,6,7,8,9,10-octahydro-1H-4,11-dithia-5,11b-diaza-benzo[a]fluoren-6-one;
(Compound 167) 2-Benzoylamino-4,5,6,7-tetrahydro-benzo[b]thiophene-3-carboxylic acid;
(Compound 168) 2-(2,2,2-Trichloro-1-phenylacetylamino-ethylamino)-4,5,6,7-tetrahydro-benzo[b]thiophene-3-carboxylic acid ethyl ester;
(Compound 169) 2-(3-Furan-2-yl-acryloylamino)-4,5,6,7-tetrahydro-benzo[b]thiophene-3-carboxylic acid amide;
(Compound 170) 3-[(3-Bromo-4-hydroxy-5-methoxy-benzylidene)-amino]-2-methyl-5,6,7,8-tetrahydro-3H-benzo[4,5]thieno[2,3-d]pyrimidin-4-one;
(Compound 171) 3-[(3-Chloro-4-hydroxy-5-methoxy-benzylidene)-amino]-2-methyl-5,6,7,8-tetrahydro-3H-benzo[4,5]thieno[2,3-d]pyrimidin-4-one;
(Compound 172) 2-[3-(4-Chloro-2-methyl-phenoxy)-propionylamino]-4,5,6,7-tetrahydro-benzo[b]thiophene-3-carboxylic acid methyl ester;
(Compound 173) 2-Amino-6-methyl-4,5,6,7-tetrahydro-benzo[b]thiophene-3-carboxylic acid isopropyl ester;
(Compound 174) 7-Benzoyloxyimino-2-pyrrol-1-yl-4,5,6,7-tetrahydro-benzo[b]thiophene-3-carboxylic acid ethyl ester;
(Compound 175) 2-(2-Formyl-pyrrol-1-yl)-4,5,6,7-tetrahydro-benzo[b]thiophene-3-carboxylic acid;
(Compound 176) 2-(5-Chloro-pentanoylamino)-4,5,6,7-tetrahydro-benzo[b]thiophene-3-carboxylic acid ethyl ester;
(Compound 177) 3-Allyl-6-methyl-2-thioxo-2,3,5,6,7,8-hexahydro-1H-benzo[4,5]thieno[2,3-d]pyrimidin-4-one;
(Compound 178) (3-Allyl-6-methyl-4-oxo-3,4,5,6,7,8-hexahydro-benzo[4,5]thieno[2,3-d]pyrimidin-2-ylsulfanyl)-acetic acid;
(Compound 179) 2-[2-(4-Bromo-phenyl-2-oxo-ethylsulfanyl)]-6-methyl-3-phenyl-5,6,7,8-tetrahydro-3H-benzo[4,5]thieno[2,3-d]pyrimidin-4-one;
(Compound 180) 2-Amino-5-methyl-4,5,6,7-tetrahydro-benzo[b]thiophene-3-carboxylic acid ethyl ester;
(Compound 181) 2-(2-Bromo-benzoylamino)-7-oxo-4,5,6,7-tetrahydro-benzo[b]thio-phene-3-carboxylic acid ethyl ester;
(Compound 182) 2-(3-Methyl-benzyolamino)-4,5,6,7-tetrahydro-benzo[b]thiophene-3-carboxylic acid ethyl ester;
(Compound 183) 2-(4-Benzoyl-benzoylamino)-4,5,6,7-tetrahydro-benzo[b]thiophene-3-carboxylic acid ethyl ester;
(Compound 184) 2-[(2-Ethoxy-benzylidene)-amino]-4,5,6,7-tetrahydro-benzo[b]thio-phene-3-carboxylic acid amide;
(Compound 185) 2-[2-Amino-3-cyano-7,7-dimethyl-4-(3-nitro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 186) 2-[2-Amino-3-cyano-7,7-dimethyl-4-(4-chloro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 187) 2-[2-Amino-3-cyano-7,7-dimethyl-4-(4-ethyl-phenyl)-5-oxo-4a,5,6,7,8,8a-hexahydro-4H-quinolin-1-yl]4,5,6,7-tetrahydro-benzo[b]thiophene-3-carboxylic acid ethyl ester;
(Compound 188) 2-[2-Amino-3-cyano-7,7-dimethyl-4-(4-nitro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 189) (3-Allyl-4-oxo-3,4,5,6,7,8-hexahydro-benzo[4,5]thieno[2,3-d]pyrimidin-2-ylsulfanyl)-acetic acid methyl ester;
(Compound 190) 2-(2-Hydroxy-ethylsulfanyl)-5,6,7,8-tetrahydro-3H-benzo[4,5]thieno[2,3-d]pyrimidin-4-one;
(Compound 191) Thiophene-2-carboxylic acid 2-ethoxy-4-(4-oxo-3,4,5,6,7,8-hexahydro-benzo-[4,5]thieno-[2,3-d]pyrimidin-2-ylsulfanyl)-phenyl ester;
(Compound 192) 2-(2-Fluoro-benzoylamino)-6-methyl-4,5,6,7-tetrahydro-benzo[b]thiophene-3-carboxylic acid isopropyl ester;
(Compound 193) 3a,7a-Dihydro-benzo[1,3]dioxole-5-carboxylic acid (3-carbamoyl-5-methyl-4,5,6,7-tetrahydro-benzo[b]thiophen-2-yl)-amide;
(Compound 194) 2-[(3a,7a-Dihydro-benzo[1,3]dioxole-5-carbonyl)-amino]-6-methyl-4,5,6,7-tetrahydro-benzo[b]thiophene-3-carboxylic acid isopropyl ester;
(Compound 195) 2-[(3a,7a-Dihydro-benzo[1,3]dioxole-5-carbonyl)-amino]-4,5,6,7-tetrahydro-benzo[b]thiophene-3-carboxylic acid isopropyl ester;
(Compound 196) 6-Methyl-2-(3-phenyl-propionylamino)-4,5,6,7-tetrahydro-benzo[b]thiophene-3-carboxylic acid amide;
(Compound 197) 2-(2,4-Dichloro-benzoylamino)-6-Methyl-4,5,6,7-tetrahydro-benzo[b]thiophene-3-carboxylic acid isopropyl ester;
(Compound 198) 2-(4-Methoxy-benzoylamino)-4,5,6,7-tetrahydro-benzo[b]thiophene-3-carboxylic acid methyl ester;
(Compound 199) Tetrahydro-fruan-2-carboxylic acid (3-carbamoyl-6-methyl-4,5,6,7-tetrahydro-benzo[b]thiophen-2-yl)-amide;
(Compound 200) 2-[2-(5-Methyl-3-nitro-pyrazol-1-yl)-acetylamino]-4,5,6,7-tetrahydro-benzo[b]thiophene-3-carboxylic acid methyl ester;
(Compound 201) 2-(4-Fluoro-benzenesulfonylamino)-4,5,6,7-tetrahydro-benzo[b]thio-phene-3-carboxylic acid amide;
(Compound 202) 6-tert-Butyl-2-(3-phenyl-3-phenylsulfanyl-propionylamino)-4,5,6,7-tetrahydro-benzo[b]thiophene-3-carboxylic acid methyl ester;
(Compound 203) 2-[2-(3,5-Dimethyl-4-nitro-pyrazol-1-yl)-acetylamino]-6-methyl-4,5,6,7-tetrahydro-benzo[b]thiophene-3-carboxylic acid ethyl ester;
(Compound 204) 2-[2-(3,5-Dimethyl-4-nitro-pyrazol-1-yl)-acetylamino]-4,5,6,7-tetrahydro-benzo[b]thiophene-3-carboxylic acid isopropyll ester;
(Compound 205) 6-Methyl-2-(3,3-diphenyl-propionylamino)-4,5,6,7-tetrahydro-benzo[b]thiophene-3-carboxylic acid ethyl ester;
(Compound 206) 2-(3,5-Dimethoxy-benzoylamino)-6-(1,1-dimethyl-propyl)-4,5,6,7-tetrahydro-benzo[b]thiophene-3-carboxylic acid methyl ester;
(Compound 207) 6-tert-Butyl-2-[2-(5-methyl-3-trifluormethyl-pyrazol-1-yl)-acetyl-amino]-4,5,6,7-tetrahydro-benzo[b]thiophene-3-carboxylic acid amide;
(Compound 208) 6-tert-Butyl-2-(3-phenyl-3-phenylsulfanyl-propionylamino)-4,5,6,7-tetrahydro-benzo[b]thiophene-3-carboxylic acid ethyl ester;
(Compound 209) 2-[2-(5-Methyl-3-nitro-pyrazol-1-yl)-acetylamino]-4,5,6,7-tetrahydro-benzo[b]thiophene-3-carboxylic acid isopropyl ester;
(Compound 210) 6-tert-Butyl-2-[2-(3-nitro-[1,2,4]triazol-1-yl)-acetylamino]-4,5,6,7-tetrahydro-benzo[b]thiophene-3-carboxylic acid methyl ester;
(Compound 211) 6-tert-Butyl-2-[2-(3,5-dimethyl-4-nitro-pyrazol-1-yl)-acetylamino]-4,5,6,7-tetrahydro-benzo[b]thiophene-3-carboxylic acid methyl ester;
(Compound 212) 6-Methyl-2-[2-(5-methyl-3-trifluoromethyl-pyrazol-1-yl)-acetylamino]-4,5,6,7-tetrahydro-benzo[b]thiophene-3-carboxylic acid ethyl ester;
(Compound 213) 6-Methyl-2-[2-(5-methyl-3-trifluoromethyl-pyrazol-1-yl)-acetylamino]-4,5,6,7-tetrahydro-benzo[b]thiophene-3-carboxylic acid methyl ester;
(Compound 214) 6-tert-Butyl-2-[2-(5-methyl-3-trifluoromethyl-pyrazol-1-yl)-acetyl-amino]-4,5,6,7-tetrahydro-benzo[b]thiophene-3-carboxylic acid ethyl ester;
(Compound 215) 6-tert-Butyl-2-[2-(3,5-dimethyl-4-nitro-pyrazol-1-yl)-acetylamino]-4,5,6,7-tetrahydro-benzo[b]thiophene-3-carboxylic acid amide;
(Compound 216) 6-tert-Butyl-2-(3-carboxy-acryloylamino)-4,5,6,7-tetrahydro-benzo[b]thiophene-3-carboxylic acid methyl ester;
(Compound 217) 6-tert-Butyl-2-(4-carboxy-butyrylamino)-4,5,6,7-tetrahydro-benzo[b]thiophene-3-carboxylic acid methyl ester;
(Compound 218) 2-[(4-Iodo-2-methyl-2H-pyrazole-3-carbonyl)-amino]-4,5,6,7-tetrahydro-benzo[b]thiophene-3-carboxylic acid ethyl ester;
(Compound 219) 2-[4-(4-Chloro-2-methyl-phenoxy)-butyrylamino]-6-methyl-4,5,6,7-tetrahydro-benzo[b]thiophene-3-carboxylic acid isopropyl ester;
(Compound 220) 2-(2-Phenyl-2-phenylsulfanyl-acetylamino)-4,5,6,7-tetrahydro-benzo[b]thiophene-3-carboxylic acid ethyl ester;
(Compound 221) 2-[(5-Methyl-3-phenyl-isoxazole-4-carbonyl)-amino]-4,5,6,7-tetrahydro-benzo[b]thiophene-3-carboxylic acid ethyl ester;
(Compound 222) 6-Methyl-2-[2-(3-nitro-[1,2,4]triazol-1-yl)-acetylamino]-4,5,6,7-tetrahydro-benzo[b]thiophene-3-carboxylic acid methyl ester;
(Compound 223) 6-Methyl-2-[2-(3-nitro-[1,2,4]triazol-1-yl)-acetylamino]-4,5,6,7-tetrahydro-benzo[b]thiophene-3-carboxylic acid isopropyl ester;
(Compound 224) 2-Amino-6-tert-butyl-4,5,6,7-tetrahydro-benzo[b]thiophene-3-carboxylic acid methyl ester;
(Compound 225) 2-Amino-6-tert-butyl-4,5,6,7-tetrahydro-benzo[b]thiophene-3-carboxylic acid amide;
(Compound 226) 6-Methyl-2-[2-(5-methyl-3-nitro-pyrazol-1-yl)-acetylamino]-4,5,6,7-tetrahydro-benzo[b]thiophene-3-carboxylic acid methyl ester;
(Compound 227) 2-[2-(3,5-Dimethyl-4-nitro-pyrazol-1-yl)-acetylamino]-4,5,6,7-tetrahydro-benzo[b]thiophene-3-carboxylic acid methyl ester;
(Compound 228) 2-[2-(3,5-Dimethyl-4-nitro-pyrazol-1-yl)-acetylamino]-4,5,6,7-tetrahydro-benzo[b]thiophene-3-carboxylic acid amide;
(Compound 229) 2-[2-(5-Methyl-3-trifluoromethyl-pyrazol-1-yl)-acetylamino]-4,5,6,7-tetrahydro-benzo[b]thiophene-3-carboxylic acid methyl ester;
(Compound 230) 6-Methyl-2-[2-(3-nitro-[1,2,4]triazol-1-yl)-acetylamino]-4,5,6,7-tetrahydro-benzo[b]thiophene-3-carboxylic acid ethyl ester;
(Compound 231) 2-[(2-Carboxy-cyclohexanecarbonyl)-amino]-4,5,6,7-tetrahydro-benzo[b]thiophene-3-carboxylic acid isopropyl ester;
(Compound 232) 2-(3-Carboxy-acryloylamino)-6-(1,1-dimethyl-propyl)-4,5,6,7-tetrahydro-benzo[b]thiophene-3-carboxylic acid amide;
(Compound 233) 6-(1,1-Dimethyl-propyl)-2-[(5-methyl-furan-2-carbonyl)-amino]-4,5,6,7-tetrahydro-benzo[b]thiophene-3-carboxylic acid amide;
(Compound 234) 6-Methyl-2-[(2-methyl-furan-3-carbonyl)-amino]-4,5,6,7-tetrahydro-benzo[b]thiophene-3-carboxylic acid isopropyl ester;
(Compound 235) 2-(4-Chloro-benzoylamino)-4,5,6,7-tetrahydro-benzo[b]thiophene-3-carboxylic acid ethyl ester;
(Compound 236) 2-Benzoylamino-4,5,6,7-tetrahydro-benzo[b]thiophene-3-carboxylic acid ethyl ester;
(Compound 237) 2-(Cyclopropanecarbonyl-amino)-4,5,6,7-tetrahydro-benzo[b]thiophene-3-carboxylic acid amide;
(Compound 238) 2-[2-(4-Nitrophenyl)-acetylamino]-4,5,6,7-tetrahydro-benzo[b]thiophene-3-carboxylic acid amide;
(Compound 239) 6-Methyl-2-[2-(3-nitro-[1,2,4]triazol-1-yl)-acetylamino]-4,5,6,7-tetrahydro-benzo[b]thiophene-3-carboxylic acid amide;
(Compound 240) Furan-2-carboxylic acid [3-(2-hydroxy-ethylcarbamoyl)-4,5,6,7-tetrahydro-benzo[b]thiophen-2-yl]-amide;
(Compound 241) Cyclopropanecarboxylic acid (3-ethoxy-4,5,6,7-tetrahydro-benzo[b]thiophen-2-yl)-amide;
(Compound 242) N-(3-Ethoxy-4,5,6,7-tetrahydro-benzo[b]thiophen-2-yl)-acet-amide;
(Compound 243) N-(3-Ethoxy-4,5,6,7-tetrahydro-benzo[b]thiophen-2-yl)-propion-amide;
(Compound 244) N-(3-Ethoxy-4,5,6,7-tetrahydro-benzo[b]thiophen-2-yl)-2-methyl-acrylamide;
(Compound 245) 3-Methyl-but-2-enoic acid (3-ethoxy-4,5,6,7-tetrahydro-benzo[b]thiophen-2-yl)-amide;
(Compound 246) But-2-enoic acid (3-ethoxy-4,5,6,7-tetrahydro-benzo[b]thiophen-2-yl)-amide;
(Compound 247) N-(3-ethoxy-4,5,6,7-tetrahydro-benzo[b]thiophen-2-yl)-2,2-dimethyl-propionamide;
(Compound 248) Thiophene-2-carboxylic acid (3-ethoxy-4,5,6,7-tetrahydro-benzo[b]thiophen-2-yl)-amide;
(Compound 249) Furan-2-carboxylic acid (3-ethoxy-4,5,6,7-tetrahydro-benzo[b]thiophen-2-yl)-amide;
(Compound 250) 2-Acetyl-amino-4,5,6,7-tetrahydrobenzo[b]thiophene-3-carboxylic acid amide;
(Compound 251) 2-(2,2,-Dimethyl-propionylamino)-4,5,6,7-tetrahydrobenzo[b]thiophene-3-carboxylic acid amide;
(Compound 252) 2-(Cyclopropanecarbonyl-amino)-4,5,6,7-tetrahydro-benzo[b]thiophene-3-carboxylic acid;
(Compound 253) Cyclopropanecarboxylic acid (3-cyano-4,5,6,7-tetrahydro-benzo[b]thiophen-2-yl)amide;
(Compound 254) 2-Amino-4,5,6,7-tetrahydro-benzo[b]thiophene-3-carbonitrile;
(Compound 255) 2-Isobutyrylamino-4,5,6,7-tetrahydrobenzo[b]thiophene-3-carboxylic acid amide;
(Compound 256) 2-Isobutyrylamino-4,5,6,7-tetrahydrobenzo[b]thiophene-3-carboxylic acid ethyl ester;
(Compound 257) 2-(Ethoxyoxalyl-amino)-4,5,6,7-tetrahydrobenzo[b]thiophene-3-carboxylic acid ethyl ester;
(Compound 258) 2-Amino-4-methyl-4,5,6,7-tetrahydrobenzo[b]thiophene-3-carboxylic acid ethyl ester;
(Compound 259) 2-(Cyclopropanecarbonyl amino)-4-methyl-4,5,6,7-tetrahydro-benzo[b]thiophene-3-carboxylic acid ethyl ester;
(Compound 260) Oxo-(4,5,6,7-tetrahydrobenzo[b]thiophen-2-yl)-acetic acid;
(Compound 261) 2-Chloro-1-(4,5,6,7-tetrahydrobenzo[b]thiophen-2-yl)-ethanone;
(Compound 262) 2-Hydrazino-1-(4,5,6,7-tetrahydrobenzo[b]thiophen-2-yl)-ethanone;
(Compound 263) 2-(2-Methyl-acryloylamino)-4,5,6,7-tetrahydrobenzo[b]thiophene-3-carboxylic acid amide;
(Compound 264) 2-[(Thiophene-2-carbonyl)-amino]-4,5,6,7-tetrahydrobenzo[b]thiophene-3-carboxylic acid amide;
(Compound 265) Furan-2-carboxylic acid (3-carbamoyl-4,5,6,7-tetrahydro-benzo[b]-thiophen-2-yl)-amide;
(Compound 266) 2-(Cyclobutanecarbonyl-amino)-4,5,6,7-tetrahydrobenzo[b]thiophene-3-carboxylic acid amide;
(Compound 267) 2-(2-Methyl-butyrylamino)-4,5,6,7-tetrahydrobenzo[b]thiophene-3-carboxylic acid amide;
(Compound 268) 2-(Cyclopropanecarbonyl-amino)-4-methyl-4,5,6,7-tetrahydrobenzo[b]thiophene-3-carboxylic acid amide;
(Compound 269) 6-tert-Butyl 2-(cyclopropanecarbonyl-amino)-4,5,6,7-tetrahydrobenzo[b]thiophene-3-carboxylic acid amide;
(Compound 270) 2-(Cyclopropanecarbonyl-amino)-6-methyl-4,5,6,7-tetrahydrobenzo[b]thiophene-3-carboxylic acid amide;
(Compound 271) 2-(Cyclopropylmethyl-amino)-4,5,6,7-tetrahydrobenzo[b]thiophene-3-carboxylic acid amide;
(Compound 272) N-(3-Cyano-4,5,6,7-tetrahydrobenzo[b]thiophen-2-yl)-acetamide;
(Compound 273) N-(3-Cyano-4,5,6,7-tetrahydrobenzo[b]thiophen-2-yl)-benzamide;
(Compound 274) 5-Bromo-furan-2-carboxylic acid (3-cyano-4,5,6,7-tetrahydrobenzo[b]thiophen-2-yl)-amide;
(Compound 275) N-(3-Cyano-4,5,6,7-tetrahydrobenzo[b]thiophen-2-yl)-2,2,2,-trifluoro-acetamide;
(Compound 276) 2-[(Furan-2-ylmethylene)-amino]-4,5,6,7-tetrahydrobenzo[b]thiophene-3-carbonitrile;
(Compound 277) N-(3-Cyano-6-methyl-4,5,6,7-tetrahydrobenzo[b]thiophen-2-yl)-acetamide;
(Compound 278) 2-[(Pyrazine-2-carbonyl)-amino]-4,5,6,7-tetrahydrobenzo[b]thiophene-3-carboxylic acid methyl ester;
(Compound 279) 2-Isobutyrylamino-4,5,6,7-tetrahydrobenzo[b]thiophene-3-carboxylic acid methyl ester;
(Compound 280) 6-Methyl-2-[(thiophene-2-carbonyl)-amino]-4,5,6,7-tetrahydrobenzo[b]thiophene-3-carboxylic acid;
(Compound 281) 2-[(Thiophene-2-carbonyl)-amino]-4,5,6,7-tetrahydrobenzo[b]thiophene-3-carboxylic acid;
(Compound 282) 2-(Cyclopropanecarbonyl-amino) 4,5,6,7-tetrahydrobenzo[b]thiophene-3-carboxylic acid;
(Compound 283) 2-(Cyclohexanecarbonyl-amino) 4,5,6,7-tetrahydrobenzo[b]thiophene-3-carboxylic acid amide;
(Compound 284) 2-Acetylamino-6-methyl-4,5,6,7-tetrahydrobenzo[b]thiophene-3-carboxylic acid amide;
(Compound 285) 2-Amino-4,7-dihydro-5H-thieno[2,3-c]-thiopyran-3-carboxylic acid amide;
(Compound 286) 2-(Cyclopropanecarbonyl-amino)-4,7-dihydro-5H-thieno[2,3-c]-thiopyran-3-carboxylic acid amide; and
(Compound 287) 2-(Cyclopropanecarbonyl-amino)-6-oxo-4,5,6,7-tetrahydro-6λ⁴-thieno[2,3-c]thiopyran-3-carboxylic acid amide.

13. A mycobacterial serine/threonine protein kinase inhibitor compound having the general formula (I): 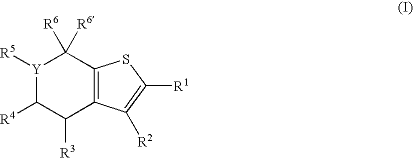
wherein
Y represents C or S;
wherein
R¹ represents R², —NH—CO—R¹⁶, —NH₂, —N═CH—R¹⁵, —N═CH—R¹⁶, —NH-CH₂—R¹⁴, —NH—CH₂—R¹⁶, —NH—SO₂—R¹⁴, —NH—CO—NH—R¹⁴, —NH—CS—NH—CH(CCl₃)—NH—CO—R¹⁶, —NH—CH(CCl₃)—NH—CO—R¹², —NH—CS—NH—R¹², —N═CH—R¹², 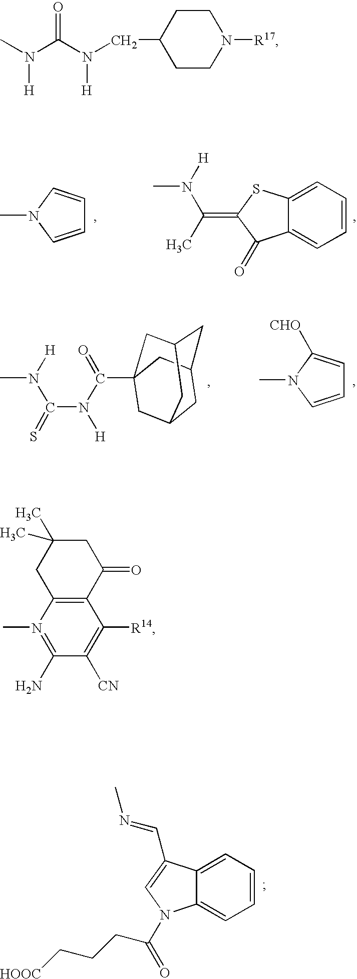
R² represents —COOR¹², —CONR¹²R¹², —CONR¹²R¹⁴, —C≡N, —COCOOR¹², —COCH₂Cl, —COCONHNH₂,
R³, R⁴, R⁵ represent independently of each other —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR¹², —SO₂R¹², —SO₃R¹², —CH₂OR¹²;
in one case R⁶ represents —R¹¹, —R¹², —R^{12′}, —OR¹², —SR¹², —NO₂, —CO—R¹², —NO, —N₃, —CN, —OCN, —NCO, —SCN, —NCS, —COOR¹², —COCN, —CONR¹²R^{12′}, —NR¹²R^{12′}, —SOR¹², —SO₂R¹², —SO₃R¹², and R⁶ is hydrogen;
in the other case R⁶ and R^{6′} together represent a carbonyl oxygen or a oximo residue ═N—OH or ═N—OOC—R¹²;
R⁷, R⁸, R⁹, R¹⁰ represent independently of each other —R¹¹, —R¹², —OR¹², —SR¹², —NO₂, —CO—R¹², —COOR¹², —OOCR¹⁶;
R¹¹ represents —F, —Cl, —Br, —I;
R¹², R^{12′} represent independently of each other —H, —CH₃, —C(R¹¹)₃, —C₂H₅, —C₃H₇, —CH(CH₃)₂, —CH₂—CH═CH₂, —CCH₃═CH₂, —CH═C(CH₃)₂, —CH═CH—CH₃, —CH═CHC₂H₅, —CH(CH₃)C₂H₅, —(CH₂)ₙ—C≡C—R⁵, —C₄H₉, —C(CH₃)₃, -Ph, —CH₂—R¹⁵; —C₂H₄OH with n being an integer from 0-2;
R¹³ represents —CF₂CHF₂, —C₅H₁₁, —C₆H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —CH₂SPh, —CH₂R¹¹, —C₂H₄R¹¹, —C₃H₆R¹¹, —C₄H₈R¹¹, —C₂H₄Ph, —CH═CH—COOR¹², —CH₂COOR¹², —C₂H₄COOR¹², —C₃H₆COOR¹², —CH(Ph)-SPh, —C₃H₅, —CH₂CH(Ph)₂, —C₄H₇, —C₅H₉, —C(CH₃)₂CH₂R¹¹, —CH₂CH(CH₃)₂, —CH(R¹¹)Ph, —CH₂CH(CH₃)CH₂C(CH₃)₃, —CH(C₂H₅)—C₄H₉, —CH(R¹¹)₂, —CH(Ph)—C₂H₅, —CH₂C(CH₃)₃,
R¹⁴ represents 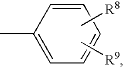
R¹⁵ represents 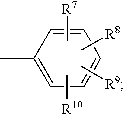
R¹⁶ is R¹², 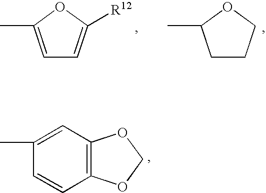
R¹³, R¹⁵, 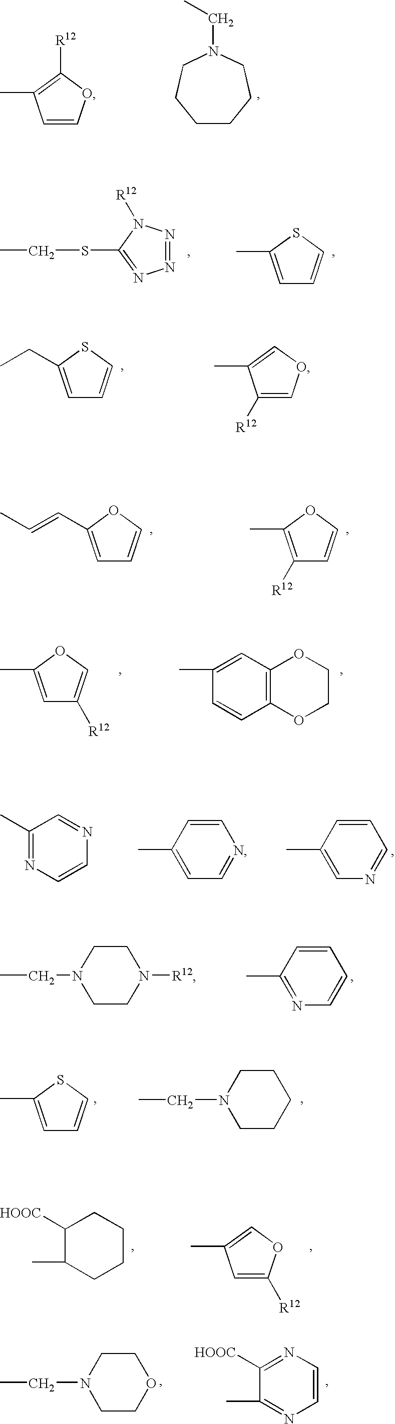 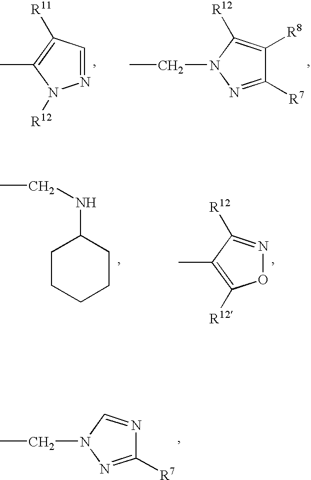
—CH₂O—R¹⁴, —CH═CH—R¹⁴, —CH₂CH(R¹⁴)—CO—R¹², —C₂H₄O—R¹⁴, —C₃H₆O—R¹⁴, —C₂H₄S—CO—R¹², —CH₂S—CO—R¹²; —COOR^{12,} 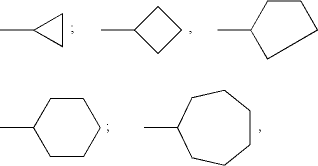
nitrobenzyl, particularly p-nitrobenzyl;
R¹⁷ represents —H, —CO—R¹², —CO—R¹³, —CO—R¹⁴, —CO—NH—R¹², —CO—NH—R¹³, —CO—NH—R¹⁴, —SO₂—R¹⁴, —CO—NH—CH₂—COO—R¹², —CO—CH₂—O—R¹⁴, —CO—CH═CH—R¹⁴, 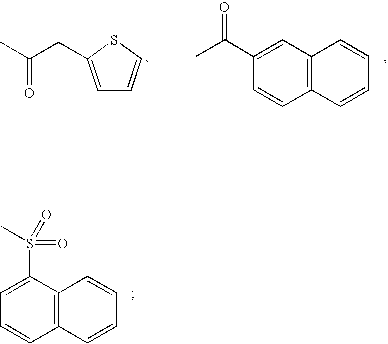
or R¹ and R² together represent a heterocyclic ring system having the following formula 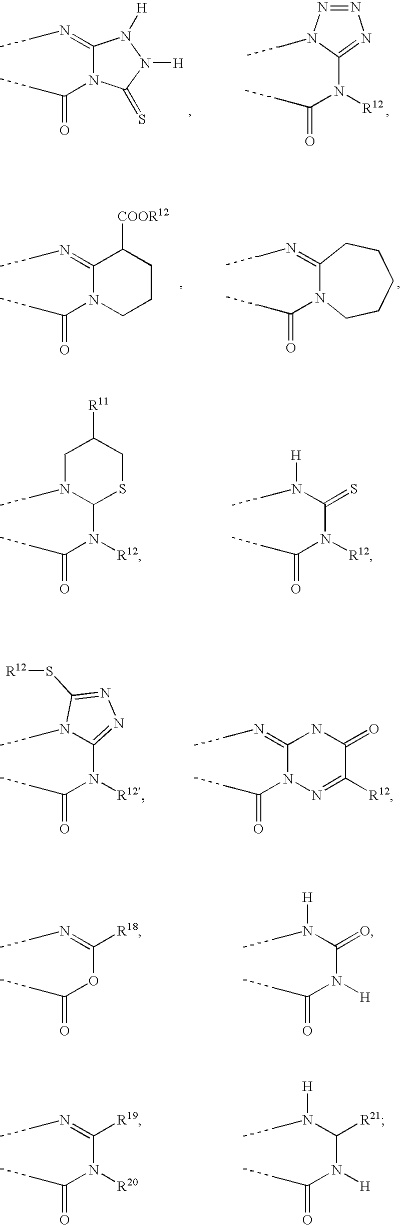
R¹⁸ represents R¹², 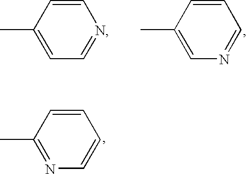
R¹⁹ represents R³, R¹⁴, —SCH₂—R³, —SCH₂—CO—R¹⁴, —SCH₂—CO—NH—R¹⁴, —SCH₂—CO—NH—CH₂—R¹², —NH—CO—CH₂—OR¹⁴, —CO—NH—N═CHR¹²(R¹⁴), 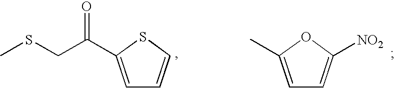
R²⁰ represents R¹², —NH—CO—R¹², —N═CH—R¹⁵;
R²¹ represents R¹⁵, 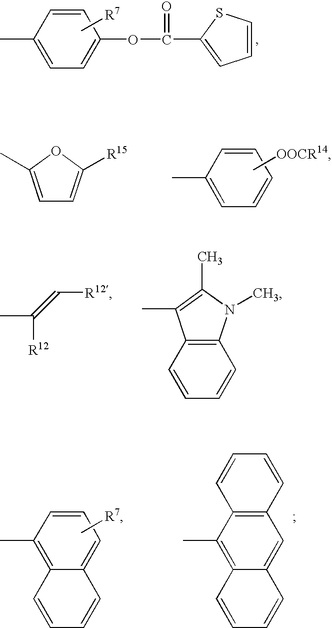
and pharmaceutically acceptable salts of these compounds.

14. A pharmaceutical composition useful for treating mycobacterial infections, said pharmaceutical composition comprising at least one inhibitor of a mycobacterial serine/threonine protein kinase according to claim 13 and a pharmaceutically acceptable carrier, excipient or diluent.

15. A pharmaceutical composition according to claim 14, wherein said mycobacterial serine/threonine protein kinase inhibitor is selected from the group consisting of:
(Compound 1) 2-{3-[1-(2-Chloro-acetyl)-piperidin-4-ylmethyl]-ureido}-4,5,6,7-tetrahydro-benzo[b]thiophene-3-carboxylic acid ethyl ester;
(Compound 2) 2-[3-(1-Butyryl-piperidin-4-ylmethyl)-ureido]-4,5,6,7-tetrahydro-benzo[b]thiophene-3-carboxylic acid ethyl ester;
(Compound 3) 2-[3-(1-Propanoyl-piperidin-4-ylmethyl)-ureido]-4,5,6,7-tetrahydro-benzo[b]thiophene-3-carboxylic acid ethyl ester;
(Compound 4) 2-[3-(1-Bemzoyl-piperidin-4-ylmethyl)-ureido]-4,5,6,7-tetrahydro-benzo[b]thiophene-3-carboxylic acid ethyl ester;
(Compound 5) 2-{3-[1-(2-Chloro-benzoyl-piperidin-4-ylmethyl]-ureido}-4,5,6,7-tetrahydro-benzo[b]thiophene-3-carboxylic acid ethyl ester;
(Compound 6) 2-[3-(1-Isobutyryl-piperidin-4-ylmethyl)-ureido]-4,5,6,7-tetrahydro-benzo[b]thiophene-3-carboxylic acid ethyl ester;
(Compound 7) 2-{3-[1-(4-Methyl-benzoyl-piperidin-4-ylmethyl]-ureido}-4,5,6,7-tetrahydro-benzo[b]thiophene-3-carboxylic acid ethyl ester;
(Compound 8) 2-[3-(1-Cyclohexanecarbonyl-piperidin-4-ylmethyl)-ureido]-4,5,6,7-tetrahydro-benzo[b]thiophene-3-carboxylic acid ethyl ester;
(Compound 9) 2-[3-(1-Cyclopropanecarbonyl-piperidin-4-ylmethyl)-ureido]-4,5,6,7-tetrahydro-benzo[b]thiophene-3-carboxylic acid ethyl ester;
(Compound 10) 2-[3-(1-Hexanoyl-piperidin-4-ylmethyl)-ureido]-4,5,6,7-tetrahydro-benzo[b]thiophene-3-carboxylic acid ethyl ester;
(Compound 11) 2-{3-[1-(2-Methyl-benzoyl-piperidin-4-ylmethyl]-ureido}-4,5,6,7-tetrahydro-benzo[b]thiophene-3-carboxylic acid ethyl ester;
(Compound 12) 2-{3-[1-(3-Chloro-2,2-dimethyl-propionyl)-piperidin-4-ylmethyl]-ureido}-4,5,6,7-tetrahydro-benzo[b]thiophene-3-carboxylic acid ethyl ester;
(Compound 13) 2-{3-[1-(3,5,5-Trimethyl-hexanoyl-piperidin-4-ylmethyl]-ureido}-4,5,6,7-tetrahydro-benzo[b]thiophene-3-carboxylic acid ethyl ester;
(Compound 14) 2-{3-[1-(2-Ethyl-hexanoyl-piperidin-4-ylmethyl]-ureido}-4,5,6,7-tetrahydro-benzo[b]thiophene-3-carboxylic acid ethyl ester;
(Compound 15) 2-{3-[1-(2-Phenyl-butyryl-piperidin-4-ylmethyl]-ureido}-4,5,6,7-tetrahydro-benzo[b]thiophene-3-carboxylic acid ethyl ester;
(Compound 16) 2-[3-(1-Cyclopentylcarbonyl-piperidin-4-ylmethyl)-ureido]-4,5,6,7-tetrahydro-benzo[b]thiophene-3-carboxylic acid ethyl ester;
(Compound 17) 2-{3-[1-(2-Chloro-2-phenyl-acetyl-piperidin-4-ylmethyl]-ureido}-4,5,6,7-tetrahydro-benzo[b]thiophene-3-carboxylic acid ethyl ester;
(Compound 18) 2-{3-[1-(4-Butyl-benzoyl-piperidin-4-ylmethyl]-ureido}-4,5,6,7-tetrahydro-benzo[b]thiophene-3-carboxylic acid ethyl ester;
(Compound 19) 2-[3-(1-Decanoyl-piperidin-4-ylmethyl)-ureido]-4,5,6,7-tetrahydro-benzo[b]thiophene-3-carboxylic acid ethyl ester;
(Compound 20) 2-[3-(1-Heptanoyl-piperidin-4-ylmethyl)-ureido]-4,5,6,7-tetrahydro-benzo[b]thiophene-3-carboxylic acid ethyl ester;
(Compound 21) 2-[3-(1-Nonanoyl-piperidin-4-ylmethyl)-ureido]-4,5,6,7-tetrahydro-benzo[b]thiophene-3-carboxylic acid ethyl ester;
(Compound 22) 2-[3-(1-Dodecanoyl-piperidin-4-ylmethyl)-ureido]-4,5,6,7-tetrahydro-benzo[b]thiophene-3-carboxylic acid ethyl ester;
(Compound 23) 2-{3-[1-(3-Methy-butyryl)-piperidin-4-ylmethyl]-ureido}-4,5,6,7-tetrahydro-benzo[b]thiophene-3-carboxylic acid ethyl ester;
(Compound 24) 2-[3-(1-Tetradecanoyl-piperidin-4-ylmethyl)-ureido]-4,5,6,7-tetrahydro-benzo[b]thiophene-3-carboxylic acid ethyl ester;
(Compound 25) 2-[3-(1-Cyclohexylcarbamoyl-piperidin-4-ylmethyl)-ureido]-4,5,6,7-tetrahydro-benzo[b]thiophene-3-carboxylic acid ethyl ester;
(Compound 26) 2-[3-(1-Phenylcarbamoyl-piperidin-4-ylmethyl)-ureido]-4,5,6,7-tetrahydro-benzo[b]thiophene-3-carboxylic acid ethyl ester;
(Compound 27) 2-[3-(1-Benzylcarbamoyl-piperidin-4-ylmethyl)-ureido]-4,5,6,7-tetrahydro-benzo[b]thiophene-3-carboxylic acid ethyl ester;
(Compound 28) 2-{3-[1-(4-Ethoxycarbonyl-phenylcarbamoyl)-piperidin-4-ylmethyl]-ureido}-4,5,6,7-tetrahydro-benzo[b]thiophene-3-carboxylic acid ethyl ester;
(Compound 29) 2-{3-[1-(3-Bromo-phenylcarbamoyl)-piperidin-4-ylmethyl]-ureido}-4,5,6,7-tetrahydro-benzo[b]thiophene-3-carboxylic acid ethyl ester;
(Compound 30) 2-{3-[1-(2-Methoxy-phenylcarbamoyl)-piperidin-4-ylmethyl]-ureido}-4,5,6,7-tetrahydro-benzo[b]thiophene-3-carboxylic acid ethyl ester;
(Compound 31) 2-{3-[1-(2-Methyl-phenylcarbamoyl)-piperidin-4-ylmethyl]-ureido}-4,5,6,7-tetrahydro-benzo[b]thiophene-3-carboxylic acid ethyl ester;
(Compound 32) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;
(Compound 33) 2-{3-[1-(4-Fluoro-benzenesulfonyl)-piperidin-4-ylmethyl]-ureido}-4,5,6,7-tetrahydro-benzo[b]thiophene-3-carboxylic acid ethyl ester;
(Compound 34) 2-{3-[1-(4-Methyl-benzenesulfonyl)-piperidin-4-ylmethyl]-ureido}-4,5,6,7-tetrahydro-benzo[b]thiophene-3-carboxylic acid ethyl ester;
(Compound 35) 2-{3-[1-Naphtalene-1-sulfonyl)-piperidin-4-ylmethyl]-ureido}-4,5,6,7-tetrahydro-benzo[b]thiophene-3-carboxylic acid ethyl ester;
(Compound 36) 2-{3-[1-(4-Chloro-benzenesulfonyl)-piperidin-4-ylmethyl]-ureido}-4,5,6,7-tetrahydro-benzo[b]thiophene-3-carboxylic acid ethyl ester;
(Compound 37) 2-{3-[1-(2,5-Dichloro-benzenesulfonyl)-piperidin-4-ylmethyl]-ureido}-4,5,6,7-tetrahydro-benzo[b]thiophene-3-carboxylic acid ethyl ester;
(Compound 38) 2-{3-[1-(2,2-Dichloro-acetyl)-piperidin-4-ylmethyl]-ureido}-4,5,6,7-tetrahydro-benzo[b]thiophene-3-carboxylic acid ethyl ester;
(Compound 39) 2-{3-[1-(3,3-Dimethy-butyryl)-piperidin-4-ylmethyl]-ureido}-4,5,6,7-tetrahydro-benzo[b]thiophene-3-carboxylic acid ethyl ester;
(Compound 40) 2-{3-[1-(Ethoxycarbonylmethyl-carbamoyl)-piperidin-4-ylmethyl]-ureido}-4,5,6,7-tetrahydro-benzo[b]thiophene-3-carboxylic acid ethyl ester;
(Compound 41) 2-{3-[1-(3-Methoxy-phenylcarbamoyl)-piperidin-4-ylmethyl]-ureido}-4,5,6,7-tetrahydro-benzo[b]thiophene-3-carboxylic acid ethyl ester;
(Compound 42) 2-{3-[1-(3,5-Bis-trifluoromethyl-benzoyl)-piperidin-4-ylmethyl]-ureido}-4,5,6,7-tetrahydro-benzo[b]thiophene-3-carboxylic acid ethyl ester;
(Compound 43) 2-[3-(1-Phenylacetyl-piperidin-4-ylmethyl]-ureido]-4,5,6,7-tetrahydro-benzo[b]thiophene-3-carboxylic acid ethyl ester;
(Compound 44) 2-{3-[1-(3,4-Dichloro-benzoyl-piperidin-4-ylmethyl]-ureido}-4,5,6,7-tetrahydro-benzo[b]thiophene-3-carboxylic acid ethyl ester;
(Compound 45) 2-{3-[1-(2,4,6-Trimethyl-benzenesulfonyl)-piperidin-4-ylmethyl]-ureido}-4,5,6,7-tetrahydro-benzo[b]thiophene-3-carboxylic acid ethyl ester;
(Compound 46) 2-{3-[1-(4-Methoxy-benzenesulfonyl)-piperidin4-ylmethyl]-ureido}-4,5,6,7-tetrahydro-benzo[b]thiophene-3-carboxylic acid ethyl ester;
(Compound 47) 2-{3-[1-(Naphtalene-2-sulfonyl)-piperidin-4-ylmethyl]-ureido}-4,5,6,7-tetrahydro-benzo[b]thiophene-3-carboxylic acid ethyl ester;
(Compound 48) 2-(3-Piperidin-4-ylmethyl-ureido)-4,5,6,7-tetrahydro-benzo[b]thiophene-3-carboxylic acid ethyl ester;
(Compound 49) 2-(3-{1-[2-(4-Chloro-phenoxy)-acetyl]-piperidin-4-ylmethyl}-ureido)-4,5,6,7-tetrahydro-benzo[b]thiophene-3-carboxylic acid ethyl ester;
(Compound 50) 2-(3-{1-[3-(4-Nitro-phenoyl)-acryloyl]-piperidin-4-ylmethyl}-ureido)-4,5,6,7-tetrahydro-benzo[b]thiophene-3-carboxylic acid ethyl ester;
(Compound 51) 2-[3-(1-Octanoyl-piperidin-4-ylmethyl)-ureido]-4,5,6,7-tetrahydro-benzo[b]thiophene-3-carboxylic acid ethyl ester;
(Compound 52) 2-[3-(1-Benzenesulfonyl-piperidin-4-ylmethyl)-ureido]-4,5,6,7-tetrahydro-benzo[b]thiophene-3-carboxylic acid ethyl ester;
(Compound 53) 2-[3-(1-Ethylylcarbamoyl-piperidin-4-ylmethyl)-ureido]-4,5,6,7-tetrahydro-benzo[b]thiophene-3-carboxylic acid ethyl ester;
(Compound 54) 2-{3-[1-(3-Fluoro-benzoyl-piperidin-4-ylmethyl]-ureido}-4,5,6,7-tetrahydro-benzo[b]thiophene-3-carboxylic acid ethyl ester;
(Compound 55) 2-{3-[1-(4-Chloro-phenylcarbamoyl)-piperidin-4-ylmethyl]-ureido}-4,5,6,7-tetrahydro-benzo[b]thiophene-3-carboxylic acid ethyl ester;
(Compound 56) 4-[3-(3-ethoxycarbonyl4,5,6,7-tetrahydro-benzo[b]thiophene-2-yl)-ureidomethyl]-piperidne-1-carboxylic acid phenyl ester;
(Compound 57) 2-{3-[1-(3-Triflluoromethyl-benzoyl-piperidin-4-ylmethyl]-ureido}-4,5,6,7-tetrahydro-benzo[b]thiophene-3-carboxylic acid ethyl ester;
(Compound 58) 2-{3-[1-(3-Bromo-benzoyl-piperidin-4-ylmethyl]-ureido}-4,5,6,7-tetrahydro-benzo[b]thiophene-3-carboxylic acid ethyl ester;
(Compound 59) 2-{3-[1-(2,4-Dichloro-phenylcarbamoyl)-piperidin-4-ylmethyl]-ureido}-4,5,6,7-tetrahydro-benzo[b]thiophene-3-carboxylic acid ethyl ester;
(Compound 60) 2-{3-[1-(2-Trifluoromethyl-phenylcarbamoyl)-piperidin-4-ylmethyl]-ureido}-4,5,6,7-tetrahydro-benzo[b]thiophene-3-carboxylic acid ethyl ester;
(Compound 61) 2-{3-[1-(4-tert-Butyl-benzoyl-piperidin-4-ylmethyl]-ureido}-4,5,6,7-tetrahydro-benzo[b]thiophene-3-carboxylic acid ethyl ester;
(Compound 62) 2-{3-[1-(3-Nitro-benzoyl-piperidin-4-ylmethyl]-ureido}-4,5,6,7-tetrahydro-benzo[b]thiophene-3-carboxylic acid ethyl ester;
(Compound 63) 2-[3-(1-Cyclobutanecarbonyl-piperidin-4-ylmethyl)-ureido]-4,5,6,7-tetrahydro-benzo[b]thiophene-3-carboxylic acid ethyl ester;
(Compound 64) 2-{3-[1-(2-Thiophen-2-ylacetyl)-piperidin-4-ylmethyl]-ureido}-4,5,6,7-tetrahydro-benzo[b]thiophene-3-carboxylic acid ethyl ester;
(Compound 65) 2-{3-[1-(Naphthalene-2-carbonyl)-piperidin-4-ylmethyl]-ureido}-4,5,6,7-tetrahydro-benzo[b]thiophene-3-carboxylic acid ethyl ester;
(Compound 66) 5,6,7,8-Tetrahydro-1H-benzo[4,5]thieno[2,3-d]pyrimidine-2,4-dione;
(Compound 67) 4-Oxo-3,4,5,6,7,8-hexahydro-benzo[4,5]thieno[2,3-d]pyrimidine-2-carboxylic acid ethyl ester;
(Compound 68) 2-Acetylamino-4,5,6,7-tetrahydro-benzo[b]thiophene-3-carboxylic acid ethyl ester;
(Compound 69) 2-Amino-4,5,6,7-tetrahydro-benzo[b]thiophene-3-carboxylic acid ethyl ester;
(Compound 70) 2-Amino-4,5,6,7-tetrahydro-benzo[b]thiophene-3-carboxylic acid amide;
(Compound 71) 2-Acetylamino-4,5,6,7-tetrahydro-benzo[b]thiophene-3-carboxylic acid;
(Compound 72) 2-(Toluene-4-sulfonylamino)-4,5,6,7-tetrahydro-benzo[b]thiophene-3-carboxylic acid amide;
(Compound 73) 7-Methyl-1,3,4,9-tetrahydro-2H-11-thia-5a,6,9,10-tetraaza-benzo[b]fluorene-5,8-dione;
(Compound 74) 7-Phenyl-1,3,4,9-tetrahydro-2H-11-thia-5a,6,9,10-tetraaza-benzo[b]fluorene-5,8-dione;
(Compound 75) 2-(5-Nitro-furan-2-yl)-2,3,5,6,7,8-hexahydro-1H-benzo[4,5]thieno[2,3-d]pyrimidin-4-one;
(Compound 76) 2-[(5-Bromo-2-hydroxy-benzylidene)-amino]-4,5,6,7-tetrahydro-benzo[b]thiophene-3-carboxylic acid amide;
(Compound 77) 2-[(4,5-Dibromo-2-hydroxy-benzylidene)-amino]-4,5,6,7-tetrahydro-benzo[b]thiophene-3-carboxylic acid ethyl ester;
(Compound 78) 2-[(2-Chloro-benzylidene)-amino]-4,5,6,7-tetrahydro-benzo[b]thiophene-3-carboxylic acid amide;
(Compound 79) 2-[1-(3-Oxo-3H-benzo[b]thiophen-2-ylidene)-ethylamino]-4,5,6,7-tetrahydro-benzo[b]thiophene-3-carboxylic ethyl ester;
(Compound 80) 2-Heptanoylamino-4,5,6,7-tetrahydro-benzo[b]thiophene-3-carboxylic acid ethyl ester;
(Compound 81) 2-(3-Bromo-benzoylamino)-4,5,6,7-tetrahydro-benzo[b]thiophene-3-carboxylic acid ethyl ester;
(Compound 82) 2-Ethyl-5,6,7,8-tetrahydro-3-oxa-9-thia-1-aza-fluoren4-one;
(Compound 83) Furan-2-carboxylic acid[3-(4-methoxy-phenylcarbamoyl)-4,5,6,7-tetrahydro-benzo[b]thiophen-2-yl]-amide;
(Compound 84) 2-Pyridin-3-yl-5,6,7,8-tetrahydro-3-oxa-9-thia-1-aza-fluoren-4-one;
(Compound 85) 2-Propionylamino-4,5,6,7-tetrahydro-benzo[b]thiophene-3-carboxylic phenylamide;
(Compound 86) 1-Allylsulfanyl-4-phenyl-6,7,8,9-tetrahydro4H-10-thia-2,3,4,10b-tetraaza-cyclopenta[a]fluoren-5-one;
(Compound 87) 2-[2-(1-Phenyl-1H-tetrazol-5-ylsulfanyl)-acetylamino]-4,5,6,7-tetrahydro-benzo[b]thiophene-3-carboxylic acid ethyl ester;
(Compound 88) 2-[3-(4-Methoxy-phenyl)-3-(2,2,2-trifluoro-acetylamino)-propionyl-amino]-4,5,6,7-tetrahydro-benzo[b]thiophene-3-carboxylic acid amide;
(Compound 89) 2-(3-Chloro-benzyolamino)-4,5,6,7-tetrahydro-benzo[b]thiophene-3-carboxylic acid ethyl ester;
(Compound 90) 2-{[1-(4-Carboxy-butyryl)-1H-indol-3-ylmethylene]-amino}-4,5,6,7-tetrahydro-benzo[b]thiophene-3-carboxylic acid ethyl ester;
(Compound 91) 2-(7-Ethyl-4-oxo-3-phenyl-3,4,5,6,7,8-hexahydro-benzo)[4,5]thieno[2,3-d]pyrimidin-2-ylsulfanyl)-N-(2-isopropoxy-phenyl)-acetamide;
(Compound 92) N, N-Diethyl-2-(7-ethyl-4-oxo-3-phenyl-3,4,5,6,7,8-hexahydro-benzo)[4,5]thieno[2,3-d]pyrimidin-2-ylsulfanyl)-acetamide;
(Compound 93) 3-[(4-Hydroxy-3-methoxy-benzylidene)-amino]-2-methyl-5,6,7,8-tetrahydro-3H-benzo[4,5]thieno[2,3-d]pyrimidin-4-one;
(Compound 94) 6-Ethyl-2-(3-phenyl-thioureido)-4,5,6,7-tetrahydro-benzo[b]thiophene-3-carboxylic acid ethyl ester;
(Compound 95) 2-[3-(Adamantane-1-carbonyl)-thioureido]-4,5,6,7-tetrahydro-benzo[b]thiophene-3-carboxylic acid ethyl ester;
(Compound 96) 2-[(3,5-Dibromo-2,4-dihydroxy-benzylidene)-amino]-4,5,6,7-tetrahydro-benzo[b]thiophene-3-carboxylic acid ethyl ester;
(Compound 97) Benzoic acid 2-ethoxy-4-(4-oxo-1,2,3,4,4a,5,6,7,8,9a-decahydro-benzo[4,5]thieno[2,3-d]pyrimidin-2-yl)-phenylester;
(Compound 98) 2-(1-Acetylamino-2,2,2-trichloro-ethylamino)-4,5,6,7-tetrahydro-benzo[b]thiophene-3-carboxylic acid ethyl ester;
(Compound 99) 2-(3,5-Di-tert-butyl-4-hydroxy-phenyl)-2,3,5,6,7,8-hexahydro-1H-benzo[4,5]thieno[2,3-d]pyrimidin-4-one;
(Compound 100) 4-Oxo-3,4,5,6,7,8-hexahydro-benzo[4,5]thieno[2,3-d]pyrimidine-2-carboxylic acid[1-(3-nitro-phenyl)-ethylidene]-hydrazide;
(Compound 101) 2-[(Pyridine-4-carbonyl)-amino]-4,5,6,7-tetrahydro-benzo[b]thio-phene-3-carboxylic acid ethyl ester;
(Compound 102) 2-(2,2,3,3-Tetrafluro-propionylamino)-4,5,6,7-tetrahydro-benzo[b]thiophene-3-carboxylic acid amide;
(Compound 103) 2-[3-(1-Acetylamino-2,2,2-trichloro-ethyl)-thioureido]-4,5,6,7-tetrahydro-benzo[b]thiophene-3-carboxylic acid ethyl ester;
(Compound 104) 2-[3-(2,2,2-Trichloro-1-propionylamino-ethyl)-thioureido]-4,5,6,7-tetrahydro-benzo[b]thiophene-3-carboxylic acid amide;
(Compound 105) 2-(3-Chloro-phenyl)-2,3,5,6,7,8-hexahydro-1H-benzo[4,5]thieno[2,3-d]pyrimidin-4-one;
(Compound 106) 2-(2-Chloro-phenyl)-2,3,5,6,7,8-hexahydro-1H-benzo[4,5]thieno[2,3-d]pyrimidin-4-one;
(Compound 107) 2-Anthracene-9-yl-2,3,5,6,7,8-hexahydro-1H-benzo[4,5]thieno[2,3-d]pyrimidin-4-one;
(Compound 108) 2-(3,5-Dibromo-2-methoxy-phenyl)-2,3,5,6,7,8-hexahydro-1H-benzo[4,5]thieno[2,3-d]pyrimidin-4-one;
(Compound 109) 2-[5-(4-Bromo-phenyl)-furan-2-yl]-2,3,4a,5,6,7,8,9a-octahydro-1H-benzo[4,5]thieno[2,3-d]pyrimidin-4-one;
(Compound 110) 2-(2,4-Dichlorophenoxy)-N-(4-oxo-2-propyl-5,6,7,8-tetrahydro-4H-benzo[4,5]thieno[2,3-d]pyrimidin-3-yl)-acetamide;
(Compound 111) 2-(3,4-Dimethoxy-benzoylamino)-4,5,6,7-tetrahydro-benzo[b]thio-phene-3-carboxylic acid ethyl ester;
(Compound 112) 2-[3-(2-Chloro-phenyl)-acryloylamino]-4,5,6,7-tetrahydro-benzo[b]thiophene-3-carboxylic acid ethyl ester;
(Compound 113) 3-(3-Ethoxycarbonyl-4,5,6,7-tetrahydro-benzo[b]thiophene-2-ylcarb-amoyl)-pyrazine-2-carboxylic acid;
(Compound 114) 2-(3-{2,2,2-Trichloro-1-[(furan-2-carbonyl)-amino]-ethyl}-thioureido)-4,5,6,7-tetrahydro-benzo[b]thiophene-3-carboxylic acid ethyl ester;
(Compound 115) 2-(1-Methyl-2-phenyl-vinyl)-2,3,4a,5,6,7,8,9a-octahydro-1H-benzo[4,5]thieno[2,3-d]pyrimidin-4-one;
(Compound 116) 2-(2-Methoxy-naphtalen-1-yl)-2,3,4a,5,6,7,8,9a-octahydro-1H-benzo[4,5]thieno[2,3-d]pyrimidin-4-one;
(Compound 117) 2-(1,2-Dimethyl-1H-indol-3-yl)-2,3,4a,5,6,7,8,9a-octahydro-1H-benzo[4,5]thieno[2,3-d]pyrimidin-4-one;
(Compound 118) 2-(Cyclohexanecarbonyl-amino)-6-methyl-4,5,6,7-tetrahydro-benzo[b]thiophene-3-carboxylic acid ethyl ester;
(Compound 119) 3-Bromo-benzoic acid 3-(4-oxo-1,2,3,4,5,6,7,8-octahydro-benzo[4,5]thieno[2,3-d]pyrimidin-2-yl)-phenyl ester;
(Compound 120) 3-Thioxo-2,3,5,6,7,8-hexahydro-1H-9-thia-1,2,3a,10-tetraaza-cyclopenta[b]fluorene4-one;
(Compound 121) 2-(2,3-Dibromo-5-ethoxy-4-hydroxy-phenyl)-2,3,5,6,7,8-hexahydro-1H-benzo[4,5]thieno[2,3-d]pyrimidin-4-one;
(Compound 122) 2-[2-(4-Chloro-phenoxy)-acetylamino]-6-methyl-4,5,6,7-tetrahydro-benzo[b]thiophene-3-carboxylic acid ethyl ester;
(Compound 123) 4-Phenyl-5a,6,7,8,9,10a-hexahydro-4H-10-thia-1,2,3,4,10b-pentaaza-cyclopenta[a]fluoren-5-one;
(Compound 124) 6-Methyl-2-[(thiophene-2-carbonyl)-amino]-4,5,6,7-tetrahydro-benzo[b]thiophene-3-carboxylic acid amide;
(Compound 125) 3-Allyl-2-(2-oxo-2-thiophen-2-yl-ethylsulfanyl)-5,6,7,8-tetrahydro-3H-benzo[4,5]thieno[2,3-d]pyrimidin-4-one;
(Compound 126) 2-[(2-Chloro-4-nitro-benzylidene)-amino]-4,5,6,7-tetrahydro-benzo[b]thiophene-3-carboxylic acid ethyl ester;
(Compound 127) 2-(2-Methyl-3,5-dinitro-benzoylamino)-4,5,6,7-tetrahydro-benzo[b]thiophene-3-carboxylic acid ethyl ester;
(Compound 128) 2-(4-Acetyl-benzoylamino)-4,5,6,7-tetrahydro-benzo[b]thiophene-3-carboxylic acid ethyl ester;
(Compound 129) 2-Benzoylamino-7-hydroxyimino-4,5,6,7-tetrahydro-benzo[b]thio-phene-3-carboxylic acid ethyl ester;
(Compound 130) 2-Formylamino-4,5,6,7-tetrahydro-benzo[b]thiophene-3-carboxylic acid ethyl ester;
(Compound 131) 5-Oxo-1,2,3,4,6,7,8,9-octahydro-5H-11-thia-5a,10-diazabenzo[b]fluorene-9-carboxylic acid ethyl ester;
(Compound 132) 2-Benzoylamino-7-oxo-4,5,6,7-tetrahydro-benzo[b]thiophene-3-carboxylic acid ethyl ester;
(Compound 133) 2-(2-Phenylsulfanyl-acetylamino)-4,5,6,7-tetrahydro-benzo[b]thio-phene-3-carboxylic acid ethyl ester;
(Compound 134) 2-(4-Nitro-benzylamino)-4,5,6,7-tetrahydro-benzo[b]thiophene-3-carboxylic acid ethyl ester;
(Compound 135) 2-[2-(4-Ethyl-piperazin-1-yl)-acetylamino]-7-oxo-4,5,6,7-tetrahydro-benzo[b]thiophene-3-carboxylic acid ethyl ester;
(Compound 136) 2-Phenylsulfanyl-3,4,7,8,9,10-hexahydro-2H,6H-12-thia-5,11-diaza-cyclohepta[b]fluorene-1,5-dione;
(Compound 137) 7-Hydroxy-2-(4-nitro-benzoylamino)-4,5,6,7-tetrahydro-benzo[b]thio-phene-3-carboxylic acid ethyl ester;
(Compound 138) 2-(2-Cyclohexylamino-acetylamino)-7-oxo-4,5,6,7-tetrahydro-benzo[b]thiophene-3-carboxylic acid ethyl ester;
(Compound 139) 2-(2-Azepan-1-yl-acetylamino)-7-oxo-4,5,6,7-tetrahydro-benzo[b]thio-phene-3-carboxylic acid ethyl ester;
(Compound 140) 7-Hydroxyimino-2-pentanoylamino-4,5,6,7-tetrahydro-benzo[b]thio-phene-3-carboxylic acid ethyl ester;
(Compound 141) 2-(2-Morpholin-4-yl-acetylamino)-7-oxo-4,5,6,7-tetrahydro-benzo[b]thiophene-3-carboxylic acid ethyl ester;
(Compound 142) 2-Benzoyloxyimion-4,5,7,8,9,10,11-heptahydro-3H-1-thia-6a,12-diaza-cyclohepta[b]fluorene-6-one;
(Compound 143) 2-(3-Acetylsulfanyl-propionylamino)-4,5,6,7-tetrahydro-benzo[b]thio-phene-3-carboxylic acid ethyl ester;
(Compound 144) 2-[3-(3,4-Dichloro-phenyl-ureido]-4,5,6,7-tetrahydro-benzo[b]thio-phene-3-carboxylic acid amide;
(Compound 145) 2-(2-Chloro-5-iodo-benzoylamino)-4,5,6,7-tetrahydro-benzo[b]thio-phene-3-carboxylic acid ethyl ester;
(Compound 146) 2-[(5-Benzyl-2-hydroxy-3-nitro-benzylidene)-amino]-4,5,6,7-tetrahydro-benzo[b]thiophene-3-carboxylic acid ethyl ester;
(Compound 147) 2-[(Pyridine-3-carbonyl)-amino]-4,5,6,7-tetrahydro-benzo[b]thiophene-3-carboxylic acid ethyl ester;
(Compound 148) 2-(3-Thiophen-2-yl-propionylamino)-4,5,6,7-tetrahydro-benzo[b]thiophene-3-carboxylic acid ethyl ester;
(Compound 149) 6-Methyl-2-(3-methyl-4-nitro-benzoylamino)-4,5,6,7-tetrahydro-benzo[b]thiophene-3-carboxylic acid ethyl ester;
(Compound 150) 2-Isopropyl-5,6,7,8-tetrahydro-3-oxa-9-thia-1-aza-fluor-4-one;
(Compound 151) 2-(2-Piperidin-1-yl-acetylamino)-4,5,6,7-tetrahydro-benzo[b]thio-phene-3-carboxylic acid amide;
(Compound 152) 3-Allyl-2-prop-2-ynylsulfanyl-5,6,7,8-tetrahydro-3H-benzo [4,5]thieno[2,3-d]pyrimidin-4-one;
(Compound 153) 2-(Trifluoromethyl)-5,6,7,8-tetrahydro-3H-benzo[4,5]thieno[2,3-d]pyrimidin-4-one;
(Compound 154) 2-[2-(4-Methyl-piperazin-1-yl)-acetylamino]-4,5,6,7-tetrahydro-benzo[b]thiophene-3-carboxylic acid amide;
(Compound 155) Furan-2-carboxylic acid-(3-phenylcarbamoyl-4,5,6,7-tetrahydro-benzo[b]thiophen-2-yl)-amide;
(Compound 156) 3-Phenyl-5,6,7,8-tetrahydro-3H-benzo[4,5]thieno[2,3-d]pyrimidin-4-one;
(Compound 157) 2-(4-Oxo-3-phenyl-3,4,5,6,7,8-hexahydro-benzo[4,5]thieno[2,3-d]pyrimidin-2-ylsulfanyl)-N-phenethyl-acetamide;
(Compound 158) 2-(3-Benzoylsulfanyl-acetylamino)-4,5,6,7-tetrahydro-benzo[b]-thiophene-3-carboxylic acid ethyl ester;
(Compound 159) 2-(5-Hydroxy-2-nitro-phenyl)-2,3,5,6,7,8-hexahydro-1H-benzo[4,5]thieno[2,3-d]pyrimidin-4-one;
(Compound 160) 2-[2-(2-Benzoylamino-2-carboxy-ethylsulfanyl)-acetylamino]-4,5,6,7-tetrahydro-benzo[b]thiophene-3-carboxylic acid ethyl ester;
(Compound 161) 2-[(5-Bromoo-2-hydroxy-benzylidene)-amino]-4,5,6,7-tetrahydro-benzo[b]thiophene-3-carboxylic acid ethyl ester;
(Compound 162) 3-[(2,4-Dihydroxy-benzylidene)-amino]-2-methyl-5,6,7,8-tetrahydro-3H-benzo[4,5]thieno[2,3-d]pyrimidin-4-one;
(Compound 163) N-(4-Oxo-2-pentyl-5-pentyl-5,6,7,8-tetrahydro-4H-benzo[4,5]thieno[2,3-d]pyrimidin-3-yl)-2-phenyl-acetamide;
(Compound 164) 2-[(2,3-Dihydro-benzo[1,4]dioxine-5-carbonyl)-amino]-4,5,6,7-tetrahydro-benzo[b]thiophene-3-carboxylic acid ethyl ester;
(Compound 165) 2-[(3,5-Dichloro-4-methoxy-benzylidene)-amino]-4,5,6,7-tetrahydro-benzo[b]thiophene-3-carboxylic acid ethyl ester;
(Compound 166) 2-Iodo-5-phenyl-2,3,4a,5,6,7,8,9,10-octahydro-1H4,11-dithia-5,11b-diaza-benzo[a]fluoren-6-one;
(Compound 167) 2-Benzoylamino-4,5,6,7-tetrahydro-benzo[b]thiophene-3-carboxylic acid;
(Compound 168) 2-(2,2,2-Trichloro-1-phenylacetylamino-ethylamino)-4,5,6,7-tetrahydro-benzo[b]thiophene-3-carboxylic acid ethyl ester;
(Compound 169) 2-(3-Furan-2-yl-acryloylamino)-4,5,6,7-tetrahydro-benzo[b]thiophene-3-carboxylic acid amide;
(Compound 170) 3-[(3-Bromo-4-hydroxy-5-methoxy-benzylidene)-amino]-2-methyl-5,6,7,8-tetrahydro-3H-benzo[4,5]thieno[2,3-d]pyrimidin-4-one;
(Compound 171) 3-[(3-Chloro-4-hydroxy-5-methoxy-benzylidene)-amino]-2-methyl-5,6,7,8-tetrahydro-3H-benzo[4,5]thieno[2,3-d]pyrimidin-4-one;
(Compound 172) 2-[3-(4-Chloro-2-methyl-phenoxy)-propionylamino]-4,5,6,7-tetrahydro-benzo[b]thiophene-3-carboxylic acid methyl ester;
(Compound 173) 2-Amino-6-methyl-4,5,6,7-tetrahydro-benzo[b]thiophene-3-carboxylic acid isopropyl ester;
(Compound 174) 7-Benzoyloxyimino-2-pyrrol-1-yl-4,5,6,7-tetrahydro-benzo[b]thiophene-3-carboxylic acid ethyl ester;
(Compound 175) 2-(2-Formyl-pyrrol-1-yl)-4,5,6,7-tetrahydro-benzo[b]thiophene-3-carboxylic acid;
(Compound 176) 2-(5-Chloro-pentanoylamino)-4,5,6,7-tetrahydro-benzo[b]thiophene-3-carboxylic acid ethyl ester;
(Compound 177) 3-Allyl-6-methyl-2-thioxo-2,3,5,6,7,8-hexahydro-1H-benzo[4,5]thieno[2,3-d]pyrimidin-4-one;
(Compound 178) (3-Allyl-6-methyl-4-oxo-3,4,5,6,7,8-hexahydro-benzo[4,5]thieno[2,3-d]pyrimidin-2-ylsulfanyl)-acetic acid;
(Compound 179) 2-[2-(4-Bromo-phenyl-2-oxo-ethylsulfanyl)]-6-methyl-3-phenyl-5,6,7,8-tetrahydro-3H-benzo[4,5]thieno[2,3-d]pyrimidin-4-one;
(Compound 180) 2-Amino-5-methyl-4,5,6,7-tetrahydro-benzo[b]thiophene-3-carboxylic acid ethyl ester;
(Compound 181) 2-(2-Bromo-benzoylamino)-7-oxo4,5,6,7-tetrahydro-benzo[b]thio-phene-3-carboxylic acid ethyl ester;
(Compound 182) 2-(3-Methyl-benzyolamino)-4,5,6,7-tetrahydro-benzo[b]thiophene-3-carboxylic acid ethyl ester;
(Compound 183) 2-(4-Benzoyl-benzoylamino)-4,5,6,7-tetrahydro-benzo[b]thiophene-3-carboxylic acid ethyl ester;
(Compound 184) 2-[(2-Ethoxy-benzylidene)-amino]-4,5,6,7-tetrahydro-benzo[b]thio-phene-3-carboxylic acid amide;
(Compound 185) 2-[2-Amino-3-cyano-7,7-dimethyl-4-(3-nitro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 186) 2-[2-Amino-3-cyano-7,7-dimethyl-4-(4-chloro-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 187) 2-[2-Amino-3-cyano-7,7-dimethyl-4-(4-ethyl-phenyl)-5-oxo-4a,5,6,7,8,8a-hexahydro-4H-quinolin-1-yl]-4,5,6,7-tetrahydro-benzo[b]thiophene-3-carboxylic acid ethyl ester;
(Compound 188) 2-[2-Amino-3-cyano-7,7-dimethyl-4-(4-nitro-phenyl )-5-oxo-4a,5,6,7,8,8a-hexahydro-4H-quinolin-1-yl]-4,5,6,7-tetrahydro-benzo[b]thiophene-3-carboxylic acid ethyl ester;
(Compound 189) (3-Allyl-4-oxo-3,4,5,6,7,8-hexahydro-benzo[4,5]thieno [2,3-d]pyrimidin-2-ylsulfanyl)-acetic acid methyl ester;
(Compound 190) 2-(2-Hydroxy-ethylsulfanyl)-5,6,7,8-tetrahydro-3H-benzo[4,5]thieno[2,3-d]pyrimidin-4-one;
(Compound 191) Thiophene-2-carboxylic acid 2-ethoxy-4-(4-oxo-3,4,5,6,7,8-hexahydro-benzo-[4,5]thieno-[2,3-d]pyrimidin-2-ylsulfanyl)-phenyl ester;
(Compound 192) 2-(2-Fluoro-benzoylamino)-6-methyl-4,5,6,7-tetrahydro-benzo[b]thiophene-3-carboxylic acid isopropyl ester;
(Compound 193) 3a,7a-Dihydro-benzo[1,3]dioxole-5-carboxylic acid (3-carbamoyl-5-methyl-4,5,6,7-tetrahydro-benzo[b]thiophen-2-yl)-amide;
(Compound 194) 2-[(3a,7a-Dihydro-benzo[1,3]dioxole-5-carbonyl)-amino]-6-methyl-4,5,6,7-tetrahydro-benzo[b]thiophene-3-carboxylic acid isopropyl ester;
(Compound 195) 2-[(3a ,7a-Dihydro-benzo[1,3]dioxole-5-carbonyl)-amino]-4,5,6,7-tetrahydro-benzo[b]thiophene-3-carboxylic acid isopropyl ester;
(Compound 196) 6-Methyl-2-(3-phenyl-propionylamino)-4,5,6,7-tetrahydro-benzo[b]thiophene-3-carboxylic acid amide;
(Compound 197) 2-(2,4-Dichloro-benzoylamino)-6-Methyl-4,5,6,7-tetrahydro-benzo[b]thiophene-3-carboxylic acid isopropyl ester;
(Compound 198) 2-(4-Methoxy-benzoylamino)-4,5,6,7-tetrahydro-benzo[b]thiophene-3-carboxylic acid methyl ester;
(Compound 199) Tetrahydro-fruan-2-carboxylic acid (3-carbamoyl-6-methyl-4,5,6,7-tetrahydro-benzo[b]thiophen-2-yl)-amide;
(Compound 200) 2-[2-(5-Methyl-3-nitro-pyrazol-1-yl)-acetylamino]-4,5,6,7-tetrahydro-benzo[b]thiophene-3-carboxylic acid methyl ester;
(Compound 201) 2-(4-Fluoro-benzenesulfonylamino)-4,5,6,7-tetrahydro-benzo[b]thio-phene-3-carboxylic acid amide;
(Compound 202) 6-tert-Butyl-2-(3-phenyl-3-phenylsulfanyl-propionylamino)-4,5,6,7-tetrahydro-benzo[b]thiophene-3-carboxylic acid methyl ester;
(Compound 203) 2-[2-(3,5-Dimethyl-4-nitro-pyrazol-1-yl)-acetylamino]-6-methyl-4,5,6,7-tetrahydro-benzo[b]thiophene-3-carboxylic acid ethyl ester;
(Compound 204) 2-[2-(3,5-Dimethyl-4-nitro-pyrazol-1-yl)-acetylamino]-4,5,6,7-tetrahydro-benzo[b]thiophene-3-carboxylic acid isopropyll ester;
(Compound 205) 6-Methyl-2-(3,3-diphenyl-propionylamino)-4,5,6,7-tetrahydro-benzo[b]thiophene-3-carboxylic acid ethyl ester;
(Compound 206) 2-(3,5-Dimethoxy-benzoylamino)-6-(1,1-dimethyl-propyl)-4,5,6,7-tetrahydro-benzo[b]thiophene-3-carboxylic acid methyl ester;
(Compound 207) 6-tert-Butyl-2-[2-(5-methyl-3-trifluormethyl-pyrazol-1-yl)-acetyl-amino]-4,5,6,7-tetrahydro-benzo[b]thiophene-3-carboxylic acid amide;
(Compound 208) 6-tert-Butyl-2-(3-phenyl-3-phenylsulfanyl-propionylamino)-4,5,6,7-tetrahydro-benzo[b]thiophene-3-carboxylic acid ethyl ester;
(Compound 209) 2-[2-(5-Methyl-3-nitro-pyrazol-1-yl)-acetylamino]-4,5,6,7-tetrahydro-benzo[b]thiophene-3-carboxylic acid isopropyl ester;
(Compound 210) 6-tert-Butyl-2-[2-(3-nitro-[1,2,4]triazol-1-yl)-acetylamino]-4,5,6,7-tetrahydro-benzo[b]thiophene-3-carboxylic acid methyl ester;
(Compound 211) 6-tert-Butyl-2-[2-(3,5-dimethyl-4-nitro-pyrazol-1-yl)-acetylamino]-4,5,6,7-tetrahydro-benzo[b]thiophene-3-carboxylic acid methyl ester;
(Compound 212) 6-Methyl-2-[2-(5-methyl-3-trifluoromethyl-pyrazol-1-yl)-acetylamino]-4,5,6,7-tetrahydro-benzo[b]thiophene-3-carboxylic acid ethyl ester;
(Compound 213) 6-Methyl-2-[2-(5-methyl-3-trifluoromethyl-pyrazol-1-yl)-acetylamino]-4,5,6,7-tetrahydro-benzo[b]thiophene-3-carboxylic acid methyl ester;
(Compound 214) 6-tert-Butyl-2-[2-(5-methyl-3-trifluoromethyl-pyrazol-1-yl)-acetyl-amino]-4,5,6,7-tetrahydro-benzo[b]thiophene-3-carboxylic acid ethyl ester;
(Compound 215) 6-tert-Butyl-2-[2-(3,5-dimethyl-4-nitro-pyrazol-1-yl)-acetylamino]-4,5,6,7-tetrahydro-benzo[b]thiophene-3-carboxylic acid amide;
(Compound 216) 6-tert-Butyl-2-(3-carboxy-acryloylamino)-4,5,6,7-tetrahydro-benzo[b]thiophene-3-carboxylic acid methyl ester;
(Compound 217) 6-tert-Butyl-2-(4-carboxy-butyrylamino)-4,5,6,7-tetrahydro-benzo[b]thiophene-3-carboxylic acid methyl ester;
(Compound 218) 2-[(4-Iodo-2-methyl-2H-pyrazole-3-carbonyl)-amino]-4,5,6,7-tetrahydro-benzo[b]thiophene-3-carboxylic acid ethyl ester;
(Compound 219) 2-[4-(4-Chloro-2-methyl-phenoxy)-butyrylamino]-6-methyl-4,5,6,7-tetrahydro-benzo[b]thiophene-3-carboxylic acid isopropyl ester;
(Compound 220) 2-(2-Phenyl-2-phenylsulfanyl-acetylamino)-4,5,6,7-tetrahydro-benzo[b]thiophene-3-carboxylic acid ethyl ester;
(Compound 221) 2-[(5-Methyl-3-phenyl-isoxazole-4-carbonyl)-amino]-4,5,6,7-tetrahydro-benzo[b]thiophene-3-carboxylic acid ethyl ester;
(Compound 222) 6-Methyl-2-[2-(3-nitro-[1,2,4]triazol-1-yl )-acetylamino]-4,5,6,7-tetrahydro-benzo[b]thiophene-3-carboxylic acid methyl ester;
(Compound 223) 6-Methyl-2-[2-(3-nitro-[1,2,4]triazol-1-yl)-acetylamino]-4,5,6,7-tetrahydro-benzo[b]thiophene-3-carboxylic acid isopropyl ester;
(Compound 224) 2-Amino-6-tert-butyl-4,5,6,7-tetrahydro-benzo[b]thiophene-3-carboxylic acid methyl ester;
(Compound 225) 2-Amino-6-tert-butyl-4,5,6,7-tetrahydro-benzo[b]thiophene-3-carboxylic acid amide;
(Compound 226) 6-Methyl-2-[2-(5-methyl-3-nitro-pyrazol-1-yl)-acetylamino]-4,5,6,7-tetrahydro-benzo[b]thiophene-3-carboxylic acid methyl ester;
(Compound 227) 2-[2-(3,5-Dimethyl-4-nitro-pyrazol-1-yl)-acetylamino]-4,5,6,7-tetrahydro-benzo[b]thiophene-3-carboxylic acid methyl ester;
(Compound 228) 2-[2-(3,5-Dimethyl-4-nitro-pyrazol-1-yl)-acetylamino]-4,5,6,7-tetrahydro-benzo[b]thiophene-3-carboxylic acid amide;
(Compound 229) 2-[2-(5-Methyl-3-trifluoromethyl-pyrazol-1-yl)-acetylamino]-4,5,6,7-tetrahydro-benzo[b]thiophene-3-carboxylic acid methyl ester;
(Compound 230) 6-Methyl-2-[2-(3-nitro-[1,2,4]triazol-1-yl )-acetylamino]-4,5,6,7-tetrahydro-benzo[b]thiophene-3-carboxylic acid ethyl ester;
(Compound 231) 2-[(2-Carboxy-cyclohexanecarbonyl)-amino]-4,5,6,7-tetrahydro-benzo[b]thiophene-3-carboxylic acid isopropyl ester;
(Compound 232) 2-(3-Carboxy-acryloylamino)-6-(1,1-dimethyl-propyl)-4,5,6,7-tetrahydro-benzo[b]thiophene-3-carboxylic acid amide;
(Compound 233) 6-(1,1-Dimethyl-propyl)-2-[(5-methyl-furan-2-carbonyl)-amino]-4,5,6,7-tetrahydro-benzo[b]thiophene-3-carboxylic acid amide;
(Compound 234) 6-Methyl-2-[(2-methyl-furan-3-carbonyl)-amino]-4,5,6,7-tetrahydro-benzo[b]thiophene-3-carboxylic acid isopropyl ester;
(Compound 235) 2-(4-Chloro-benzoylamino)-4,5,6,7-tetrahydro-benzo[b]thiophene-3-carboxylic acid ethyl ester;
(Compound 236) 2-Benzoylamino-4,5,6,7-tetrahydro-benzo[b]thiophene-3-carboxylic acid ethyl ester;
(Compound 237) 2-(Cyclopropanecarbonyl-amino)-4,5,6,7-tetrahydro-benzo[b]thiophene-3-carboxylic acid amide;
(Compound 238) 2-[2-(4-Nitrophenyl)-acetylamino]-4,5,6,7-tetrahydro-benzo[b]thiophene-3-carboxylic acid amide;
(Compound 239) 6-Methyl-2-[2-(3-nitro-[1,2,4]triazol-1-yl)-acetylamino]-4,5,6,7-tetrahydro-benzo[b]thiophene-3-carboxylic acid amide;
(Compound 240) Furan-2-carboxylic acid[3-(2-hydroxy-ethylcarbamoyl)-4,5,6,7-tetrahydro-benzo[b]thiophen-2-yl]-amide;
(Compound 241) Cyclopropanecarboxylic acid (3-ethoxy-4,5,6,7-tetrahydro-benzo[b]thiophen-2-yl)-amide;
(Compound 242) N-(3-Ethoxy-4,5,6,7-tetrahydro-benzo[b]thiophen-2-yl)-acetamide;
(Compound 243) N-(3-Ethoxy-4,5,6,7-tetrahydro-benzo[b]thiophen-2-yl)-propion-amide;
(Compound 244) N-(3-Ethoxy-4,5,6,7-tetrahydro-benzo[b]thiophen-2-yl)-2-methyl-acrylamide;
(Compound 245) 3-Methyl-but-2-enoic acid (3-ethoxy-4,5,6,7-tetrahydro-benzo[b]thiophen-2-yl)-amide;
(Compound 246) But-2-enoic acid (3-ethoxy-4,5,6,7-tetrahydro-benzo[b]thiophen-2-yl)-amide;
(Compound 247) N-(3-ethoxy-4,5,6,7-tetrahydro-benzo[b]thiophen-2-yl)-2,2-dimethyl-propionamide;
(Compound 248) Thiophene-2-carboxylic acid (3-ethoxy-4,5,6,7-tetrahydro-benzo[b]thiophen-2-yl)-amide;
(Compound 249) Furan-2-carboxylic acid (3-ethoxy-4,5,6,7-tetrahydro-benzo[b]thiophen-2-yl)-amide;
(Compound 250) 2-Acetyl-amino-4,5,6,7-tetrahydrobenzo[b]thiophene-3-carboxylic acid amide;
(Compound 251) 2-(2,2,-Dimethyl-propionylamino)-4,5,6,7-tetrahydrobenzo[b]thiophene-3-carboxylic acid amide;
(Compound 252) 2-(Cyclopropanecarbonyl-amino)-4,5,6,7-tetrahydro-benzo[b]thiophene-3-carboxylic acid;
(Compound 253) Cyclopropanecarboxylic acid (3-cyano4,5,6,7-tetrahydro-benzo[b]thiophen-2-yl) amide;
(Compound 254) 2-Amino-4,5,6,7-tetrahydro-benzo[b]thiophene-3-carbonitrile;
(Compound 255) 2-Isobutyrylamino-4,5,6,7-tetrahydrobenzo[b]thiophene-3-carboxylic acid amide;
(Compound 256) 2-lsobutyrylamino-4,5,6,7-tetrahydrobenzo[b]thiophene-3-carboxylic acid ethyl ester;
(Compound 257) 2-(Ethoxyoxalyl-amino)-4,5,6,7-tetrahydrobenzo[b]thiophene-3-carboxylic acid ethyl ester;
(Compound 258) 2-Amino-4-methyl-4,5,6,7-tetrahydrobenzo[b]thiophene-3-carboxylic acid ethyl ester;
(Compound 259) 2-(Cyclopropanecarbonyl amino)-4-methyl-4,5,6,7-tetrahydro-benzo[b]thiophene-3-carboxylic acid ethyl ester;
(Compound 260) Oxo-(4,5,6,7-tetrahydrobenzo[b]thiophen-2-yl)-acetic acid;
(Compound 261) 2-Chloro-1-(4,5,6,7-tetrahydrobenzo[b]thiophen-2-yl)-ethanone;
(Compound 262) 2-Hydrazino-1-(4,5,6,7-tetrahydrobenzo[b]thiophen-2-yl)-ethanone;
(Compound 263) 2-(2-Methyl-acryloylamino)-4,5,6,7-tetrahydrobenzo[b]thiophene-3-carboxylic acid amide;
(Compound 264) 2-[(Thiophene-2-carbonyl)-amino]-4,5,6,7-tetrahydrobenzo[b]thiophene-3-carboxylic acid amide;
(Compound 265) Furan-2-carboxylic acid(3-carbamoyl-4,5,6,7-tetrahydro-benzo[b]-thiophen-2-yl)-amide;
(Compound 266) 2-(Cyclobutanecarbonyl-amino)-4,5,6,7-tetrahydrobenzo[b]thiophene-3-carboxylic acid amide;
(Compound 267) 2-(2-Methyl-butyrylamino)-4,5,6,7-tetrahydrobenzo[b]thiophene-3-carboxylic acid amide;
(Compound 268) 2-(Cyclopropanecarbonyl-amino)-4-methyl-4,5,6,7-tetrahydrobenzo[b]thiophene-3-carboxylic acid amide;
(Compound 269) 6-tert-Butyl 2-(cyclopropanecarbonyl-amino)-4,5,6,7-tetrahydrobenzo[b]thiophene-3-carboxylic acid amide;
(Compound 270) 2-(Cyclopropanecarbonyl-amino)-6-methyl-4,5,6,7-tetrahydrobenzo[b]thiophene-3-carboxylic acid amide;
(Compound 271) 2-(Cyclopropylmethyl-amino)-4,5,6,7-tetrahydrobenzo[b]thiophene-3-carboxylic acid amide;
(Compound 272) N-(3-Cyano-4,5,6,7-tetrahydrobenzo[b]thiophen-2-yl)-acetamide;
(Compound 273) N-(3-Cyano-4, 5,6,7-tetrahydrobenzo[b]thiophen-2-yl)-benzamide;
(Compound 274) 5-Bromo-furan-2-carboxylic acid (3-cyano-4,5,6,7-tetrahydrobenzo[b]thiophen-2-yl)-amide;
(Compound 275) N-(3-Cyano-4,5,6,7-tetrahydrobenzo[b]thiophen-2-yl)-2,2,2,-trifluoro-acetamide;
(Compound 276) 2-[(Furan-2-ylmethylene)-amino]-4,5,6,7-tetrahydrobenzo[b]thiophene-3-carbonitrile;
(Compound 277) N-(3-Cyano-6-methyl-4,5,6,7-tetrahydrobenzo[b]thiophen-2-yl)-acetamide;
(Compound 278) 2-[(Pyrazine-2-carbonyl)-amino]-4,5,6,7-tetrahydrobenzo[b]thiophene-3-carboxylic acid methyl ester;
(Compound 279) 2-Isobutyrylamino-4,5,6,7-tetrahydrobenzo[b]thiophene-3-carboxylic acid methyl ester;
(Compound 280) 6-Methyl-2-[(thiophene-2-carbonyl)-amino]-4,5,6,7-tetrahydrobenzo[b]thiophene-3-carboxylic acid;
(Compound 281) 2-[(Thiophene-2-carbonyl)-amino]-4,5,6,7-tetrahydrobenzo[b]thiophene-3-carboxylic acid;
(Compound 282) 2-(Cyclopropanecarbonyl-amino)4,5,6,7-tetrahydrobenzo[b]thiophene-3-carboxylic acid;
(Compound 283) 2-(Cyclohexanecarbonyl-amino)4,5,6,7-tetrahydrobenzo[b]thiophene-3-carboxylic acid amide;
(Compound 284) 2-Acetylamino-6-methyl-4,5,6,7-tetrahydrobenzo[b]thiophene-3-carboxylic acid amide;
(Compound 285) 2-Amino-4,7-dihydro-5H-thieno[2,3-c]-thiopyran-3-carboxylic acid amide;
(Compound 286) 2-(Cyclopropanecarbonyl-amino)-4,7-dihydro-5H-thieno[2,3-c]-thiopyran-3-carboxylic acid amide; and
(Compound 287) 2-(Cyclopropanecarbonyl-amino)-6-oxo-4,5,6,7-tetrahydro-6λ⁴-thieno[2,3-c]thiopyran-3-carboxylic acid amide,
in a pharmaceutically acceptable carrier.

16. A method of identifying compounds useful for the treatment of mycobacterial infections, said method comprising:
providing a mycobacterial serine/threonine protein kinase involved in mycobacterial virulence;
contacting said mycobacterium serine/threonine protein kinase with a test compound;
measuring the kinase activity of said mycobacterium serine/threonine protein kinase in the presence of said test compound, wherein inhibition of said kinase activity in the presence of said test compound indicates said test compound is useful for treating mycobacterial infections.

17. The method according to claim 16, wherein said mycobacterial serine/threonine protein kinase is from Mycobacterium tuberculosis, M. bovis, M. africanum, M. leprae, M. abscessus, M. avium, M. celatum, M. chelonae, M. fortuitum, M. genavense, M. gordonae, M. haemophilum, M. intracellulare, M. kansii, M. malmoense, M. marinum, M. scrofulaceum, M. simiae, M. szulgai, M. ulcerans and/or M. xenopi.

18. The method according to claim 17, wherein said mycobacterial serine/threonine protein kinase is selected from the group consisting of PknG, PknB, and PknH.

19. The method according to claim 18, wherein said mycobacterial serine/threonine protein kinase is PknG.

20. The method according to claim 18, wherein said mycobacterial serine/threonine protein kinase is PknB.

21. The method according to claim 18, wherein said mycobacterial serine/threonine protein kinase is PknH.

22. A mycobacterial serine/threonine protein kinase inhibiting compound having the formula (II): 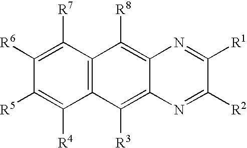
wherein:
R¹ and R² are independently of each other —(CH₂)ₚ—NH—(CH₂)ₙ—R⁹, —(CH₂)ₛ—S—(CH₂)ₘR¹⁰, —(CH₂)ₘ—O—(CH₂)ₚ—R¹¹, —(CH₂)ᵣR³, —CH═CH—R¹¹, —(CH₂)ₘ—CH(OH)—(CH₂)ₚ—R¹¹, —(CH₂)_{q}—R¹¹, —R⁹, —R¹⁰, —R¹², —R¹³, 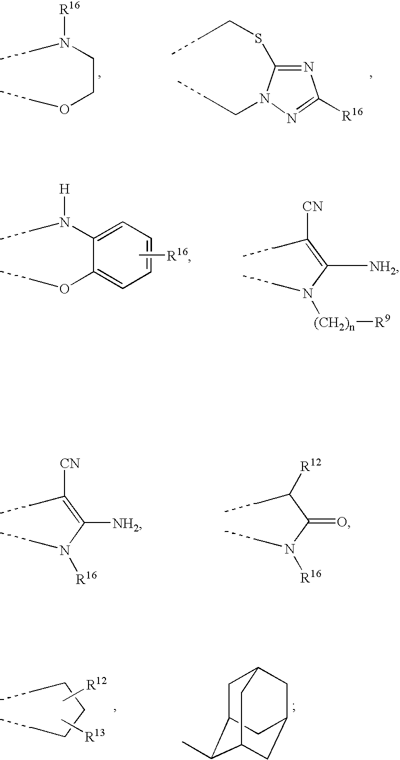
R³, R⁴, R⁵, R⁶, R⁷, and R⁸ are independently of each other —H, , —Cl, —Br, —I, —SO₃H, —SO₃NH₂, —(CH₂)_{S}—COOR¹⁶, —(CH₂)ₚ—COOR¹⁷, —OR¹⁶, —SR¹⁶, —NR¹⁶R¹⁷, —OOCR¹⁶, —OOCR¹⁷, —NH—CO—R¹⁶, —NH—CO—R¹⁷, —CO—NH—R¹⁶, —CO—NH—R¹⁷, —NO₂, —N₃, —CN, —OCN, —NCO, —SCN, —NCS, —CO—R¹⁶, —CO—R¹⁷, —COCN, —CONR¹⁶R¹⁷, —SOR¹⁶, —SOR¹⁷, —SO₂R¹⁶, —SO₂R¹⁷, —SO₃R¹⁶, —SO₃R¹⁷, —OCF₃;
R⁹, R¹⁰, and R¹¹ are independently of each other —CN, —NR¹⁶R¹⁷, —NHR¹⁶, —NHR¹⁷, 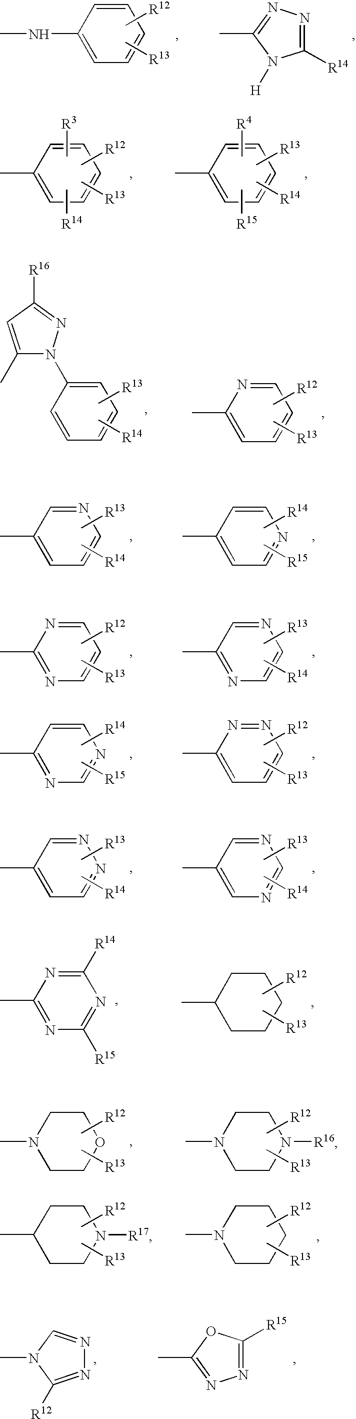 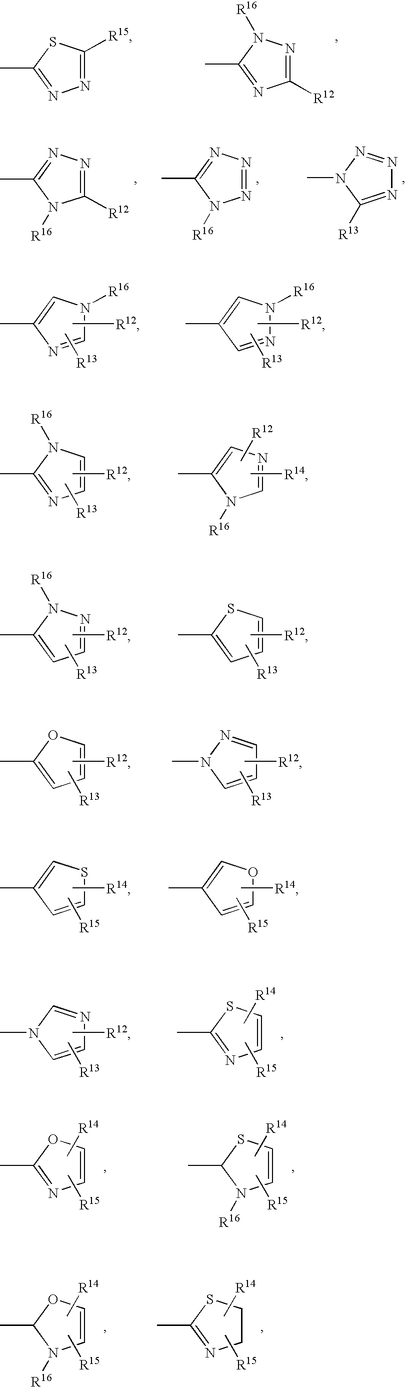 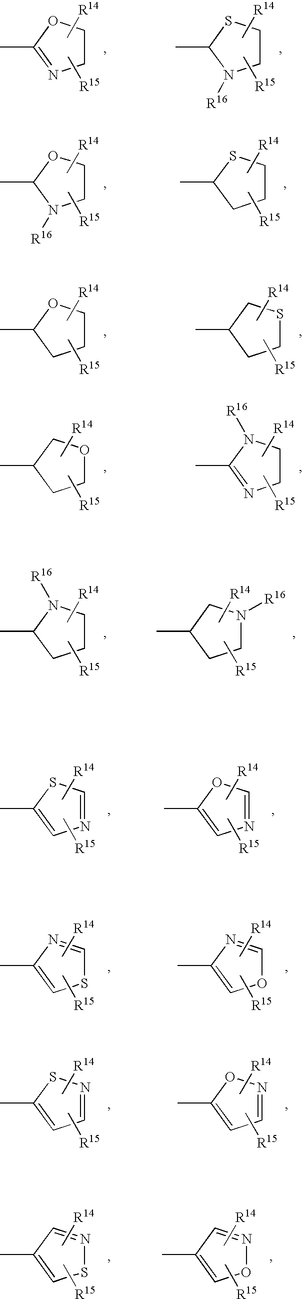 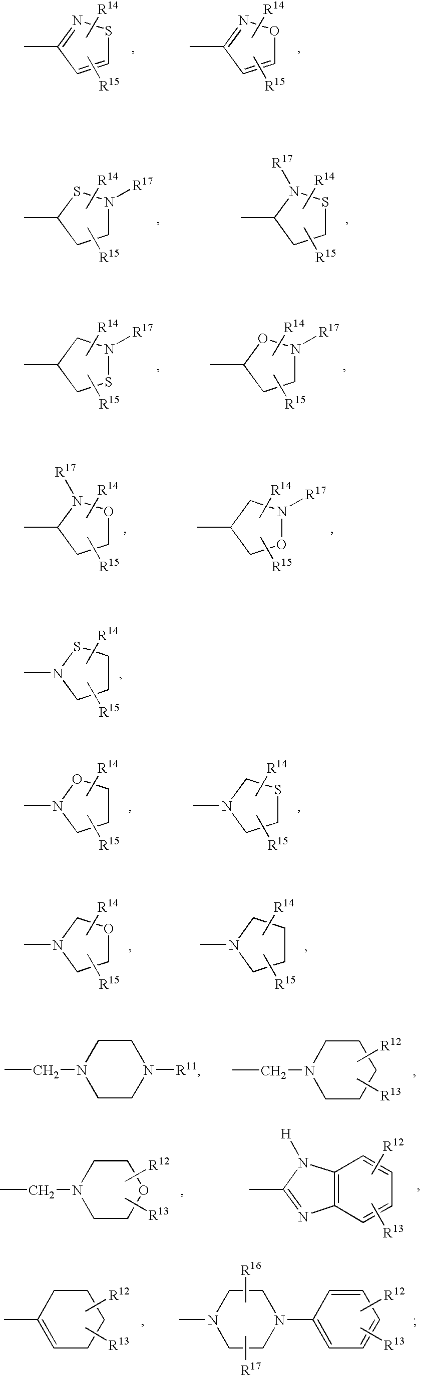
R^{12 l , R}¹³, R¹⁴, and R¹⁵ are independently of each other —R³, —R⁴, —R⁵, —R⁶, —R¹⁶, —R¹⁷, —CH(COOR¹⁶)(COOR¹⁷), —CH(CN)(COOR¹⁶), 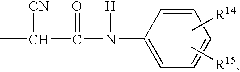
R¹⁶ and R¹⁷ are independently of each other —H, —CH₃, —C₂H₅, —C₃H₇, —CH(CH₃)₂, —C₄H₉, —C₅H₁₃, —C₆H₁₃, -cyclo-C₆H₁₁, -cyclo-C₅H₉, -Cyclo-C₄H₇, -cyclo-C₃H₅, —(CH₂)ᵣCH(CH₃)₂, —CH(CH₃)C₂H₅, —C(CH₃)₃, —CH═CH₂, —CH₂—CH═CH₂, -Ph, —CH₂Ph, —C₂H₄Ph, —CH(CN)₂, —CF₃, —CCl₃, —CBr₃, —C₂F₅, —(CH₂)ᵣ—OH, —CH₂F, —CH₂Cl, —CH₂Br, —CH₂I, —CHF₂, —CHCl₂, —CHBr₂, —(CH₂)ᵣ—SH, —C₆H₄—CH₃, —C₆H₃(CH₃)₂, 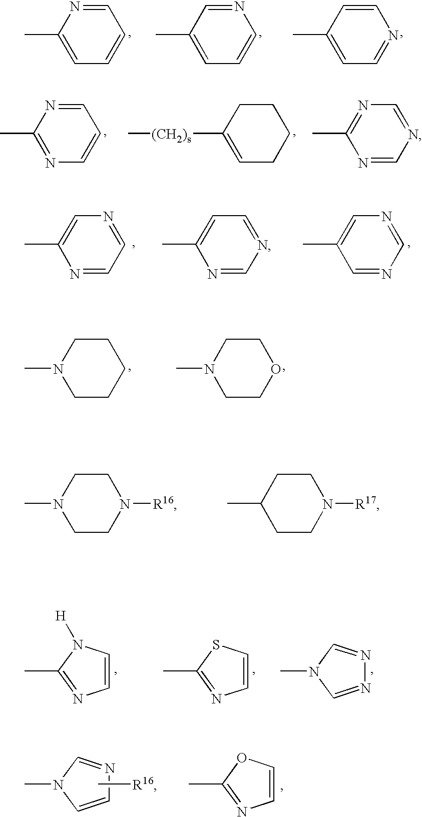
m, n, p, q, s are independently of each other integer from 0-6,
r is an integer from 1-6,
and the corresponding N-oxides in position 1 and/or 4 of these compounds; and the corresponding reduced forms of these compounds wherein the double bond in position 1 and/or 3 is hydrogenated; and pharmaceutically acceptable salts of these compounds.

23. A method for treating mycobacterial infections comprising: administering to a subject in need of such treatment a composition comprising a compound according to claim 22.
